SEQUENCE LISTING (298 sequences)

SEQ ID NO: 1 - DNA, 14 bp, artificial
  Oligonucleotide nucleobase seqeunce
gaaaaacataacaa

SEQ ID NO: 2 - DNA, 14 bp, artificial
  Oligonucleotide nucleobase seqeunce
agaaaaacataaca

SEQ ID NO: 3 - DNA, 14 bp, artificial
  Oligonucleotide nucleobase seqeunce
aagaaaaacataac

SEQ ID NO: 4 - DNA, 14 bp, artificial
  Oligonucleotide nucleobase seqeunce
aaagaaaaacataa

SEQ ID NO: 5 - DNA, 14 bp, artificial
  Oligonucleotide nucleobase seqeunce
aaaagaaaaacata

SEQ ID NO: 6 - DNA, 14 bp, artificial
  Oligonucleotide nucleobase seqeunce
aaaaagaaaaacat

SEQ ID NO: 7 - DNA, 14 bp, artificial
  Oligonucleotide nucleobase seqeunce
aaaaaagaaaaaca

SEQ ID NO: 8 - DNA, 14 bp, artificial
  Oligonucleotide nucleobase seqeunce
aaaaaaagaaaaac

SEQ ID NO: 9 - DNA, 14 bp, artificial
  Oligonucleotide nucleobase seqeunce
gaaaaaaagaaaaa

SEQ ID NO: 10 - DNA, 14 bp, artificial
  Oligonucleotide nucleobase seqeunce
tgaaaaaaagaaaa

SEQ ID NO: 11 - DNA, 14 bp, artificial
  Oligonucleotide nucleobase seqeunce
ctgaaaaaaagaaa

SEQ ID NO: 12 - DNA, 14 bp, artificial
  Oligonucleotide nucleobase seqeunce
tctgaaaaaaagaa

SEQ ID NO: 13 - DNA, 14 bp, artificial
  Oligonucleotide nucleobase seqeunce
gtctgaaaaaaaga

SEQ ID NO: 14 - DNA, 14 bp, artificial
  Oligonucleotide nucleobase seqeunce
tgtctgaaaaaaag

SEQ ID NO: 15 - DNA, 14 bp, artificial
  Oligonucleotide nucleobase seqeunce
gtgtctgaaaaaaa

SEQ ID NO: 16 - DNA, 14 bp, artificial
  Oligonucleotide nucleobase seqeunce
ggtgtctgaaaaaa

SEQ ID NO: 17 - DNA, 14 bp, artificial
  Oligonucleotide nucleobase seqeunce
gggtgtctgaaaaa

SEQ ID NO: 18 - DNA, 14 bp, artificial
  Oligonucleotide nucleobase seqeunce
agggtgtctgaaaa

SEQ ID NO: 19 - DNA, 14 bp, artificial
  Oligonucleotide nucleobase seqeunce
aagggtgtctgaaa

SEQ ID NO: 20 - DNA, 14 bp, artificial
  Oligonucleotide nucleobase seqeunce
caagggtgtctgaa

SEQ ID NO: 21 - DNA, 14 bp, artificial
  Oligonucleotide nucleobase seqeunce
caagggtgtctgaa

SEQ ID NO: 22 - DNA, 14 bp, artificial
  Oligonucleotide nucleobase seqeunce
ccaagggtgtctga

SEQ ID NO: 23 - DNA, 14 bp, artificial
  Oligonucleotide nucleobase seqeunce
ccaagggtgtctga

SEQ ID NO: 24 - DNA, 14 bp, artificial
  Oligonucleotide nucleobase seqeunce
ccaagggtgtctga

SEQ ID NO: 25 - DNA, 14 bp, artificial
  Oligonucleotide nucleobase seqeunce
tccaagggtgtctg

SEQ ID NO: 26 - DNA, 14 bp, artificial
  Oligonucleotide nucleobase seqeunce
tccaagggtgtctg

SEQ ID NO: 27 - DNA, 14 bp, artificial
  Oligonucleotide nucleobase seqeunce
tccaagggtgtctg

SEQ ID NO: 28 - DNA, 14 bp, artificial
  Oligonucleotide nucleobase seqeunce
ctccaagggtgtct

SEQ ID NO: 29 - DNA, 14 bp, artificial
  Oligonucleotide nucleobase seqeunce
ctccaagggtgtct

SEQ ID NO: 30 - DNA, 14 bp, artificial
  Oligonucleotide nucleobase seqeunce
ctccaagggtgtct

SEQ ID NO: 31 - DNA, 8 bp, artificial
  Oligonucleotide nucleobase seqeunce
gggtgtct

SEQ ID NO: 32 - DNA, 14 bp, artificial
  Oligonucleotide nucleobase seqeunce
actccaagggtgtc

SEQ ID NO: 33 - DNA, 14 bp, artificial
  Oligonucleotide nucleobase seqeunce
actccaagggtgtc

SEQ ID NO: 34 - DNA, 14 bp, artificial
  Oligonucleotide nucleobase seqeunce
actccaagggtgtc

SEQ ID NO: 35 - DNA, 14 bp, artificial
  Oligonucleotide nucleobase seqeunce
aactccaagggtgt

SEQ ID NO: 36 - DNA, 14 bp, artificial
  Oligonucleotide nucleobase seqeunce
aactccaagggtgt

SEQ ID NO: 37 - DNA, 14 bp, artificial
  Oligonucleotide nucleobase seqeunce
aactccaagggtgt

SEQ ID NO: 38 - DNA, 14 bp, artificial
  Oligonucleotide nucleobase seqeunce
aaactccaagggtg

SEQ ID NO: 39 - DNA, 14 bp, artificial
  Oligonucleotide nucleobase seqeunce
aaaactccaagggt

SEQ ID NO: 40 - DNA, 14 bp, artificial
  Oligonucleotide nucleobase seqeunce
gaaaactccaaggg

SEQ ID NO: 41 - DNA, 14 bp, artificial
  Oligonucleotide nucleobase seqeunce
agaaaactccaagg

SEQ ID NO: 42 - DNA, 14 bp, artificial
  Oligonucleotide nucleobase seqeunce
agaaaccaattaga

SEQ ID NO: 43 - DNA, 14 bp, artificial
  Oligonucleotide nucleobase seqeunce
aagaaaccaattag

SEQ ID NO: 44 - DNA, 14 bp, artificial
  Oligonucleotide nucleobase seqeunce
gaagaaaccaatta

SEQ ID NO: 45 - DNA, 14 bp, artificial
  Oligonucleotide nucleobase seqeunce
agaagaaaccaatt

SEQ ID NO: 46 - DNA, 14 bp, artificial
  Oligonucleotide nucleobase seqeunce
gagaagaaaccaat

SEQ ID NO: 47 - DNA, 14 bp, artificial
  Oligonucleotide nucleobase seqeunce
tgagaagaaaccaa

SEQ ID NO: 48 - DNA, 14 bp, artificial
  Oligonucleotide nucleobase seqeunce
gtgagaagaaacca

SEQ ID NO: 49 - DNA, 14 bp, artificial
  Oligonucleotide nucleobase seqeunce
tgtgagaagaaacc

SEQ ID NO: 50 - DNA, 14 bp, artificial
  Oligonucleotide nucleobase seqeunce
gtgtgagaagaaac

SEQ ID NO: 51 - DNA, 14 bp, artificial
  Oligonucleotide nucleobase seqeunce
tgtgtgagaagaaa

SEQ ID NO: 52 - DNA, 14 bp, artificial
  Oligonucleotide nucleobase seqeunce
ctgtgtgagaagaa

SEQ ID NO: 53 - DNA, 14 bp, artificial
  Oligonucleotide nucleobase seqeunce
actgtgtgagaaga

SEQ ID NO: 54 - DNA, 14 bp, artificial
  Oligonucleotide nucleobase seqeunce
aactgtgtgagaag

SEQ ID NO: 55 - DNA, 14 bp, artificial
  Oligonucleotide nucleobase seqeunce
gaactgtgtgagaa

SEQ ID NO: 56 - DNA, 14 bp, artificial
  Oligonucleotide nucleobase seqeunce
ggaactgtgtgaga

SEQ ID NO: 57 - DNA, 14 bp, artificial
  Oligonucleotide nucleobase seqeunce
tggaactgtgtgag

SEQ ID NO: 58 - DNA, 14 bp, artificial
  Oligonucleotide nucleobase seqeunce
atggaactgtgtga

SEQ ID NO: 59 - DNA, 14 bp, artificial
  Oligonucleotide nucleobase seqeunce
tatggaactgtgtg

SEQ ID NO: 60 - DNA, 14 bp, artificial
  Oligonucleotide nucleobase seqeunce
tatggaactgtgtg

SEQ ID NO: 61 - DNA, 14 bp, artificial
  Oligonucleotide nucleobase seqeunce
ctatggaactgtgt

SEQ ID NO: 62 - DNA, 14 bp, artificial
  Oligonucleotide nucleobase seqeunce
actatggaactgtg

SEQ ID NO: 63 - DNA, 8 bp, artificial
  Oligonucleotide nucleobase seqeunce
gaactgtg

SEQ ID NO: 64 - DNA, 14 bp, artificial
  Oligonucleotide nucleobase seqeunce
cactatggaactgt

SEQ ID NO: 65 - DNA, 8 bp, artificial
  Oligonucleotide nucleobase seqeunce
ggaactgt

SEQ ID NO: 66 - DNA, 14 bp, artificial
  Oligonucleotide nucleobase seqeunce
gcactatggaactg

SEQ ID NO: 67 - DNA, 14 bp, artificial
  Oligonucleotide nucleobase seqeunce
gcactatggaactg

SEQ ID NO: 68 - DNA, 14 bp, artificial
  Oligonucleotide nucleobase seqeunce
gcactatggaactg

SEQ ID NO: 69 - DNA, 8 bp, artificial
  Oligonucleotide nucleobase seqeunce
tggaactg

SEQ ID NO: 70 - DNA, 14 bp, artificial
  Oligonucleotide nucleobase seqeunce
agcactatggaact

SEQ ID NO: 71 - DNA, 14 bp, artificial
  Oligonucleotide nucleobase seqeunce
agcactatggaact

SEQ ID NO: 72 - DNA, 8 bp, artificial
  Oligonucleotide nucleobase seqeunce
atggaact

SEQ ID NO: 73 - DNA, 14 bp, artificial
  Oligonucleotide nucleobase seqeunce
aagcactatggaac

SEQ ID NO: 74 - DNA, 8 bp, artificial
  Oligonucleotide nucleobase seqeunce
tatggaac

SEQ ID NO: 75 - DNA, 14 bp, artificial
  Oligonucleotide nucleobase seqeunce
taagcactatggaa

SEQ ID NO: 76 - DNA, 8 bp, artificial
  Oligonucleotide nucleobase seqeunce
ctatggaa

SEQ ID NO: 77 - DNA, 14 bp, artificial
  Oligonucleotide nucleobase seqeunce
ataagcactatgga

SEQ ID NO: 78 - DNA, 8 bp, artificial
  Oligonucleotide nucleobase seqeunce
actatgga

SEQ ID NO: 79 - DNA, 14 bp, artificial
  Oligonucleotide nucleobase seqeunce
cataagcactatgg

SEQ ID NO: 80 - DNA, 8 bp, artificial
  Oligonucleotide nucleobase seqeunce
cactatgg

SEQ ID NO: 81 - DNA, 14 bp, artificial
  Oligonucleotide nucleobase seqeunce
ccataagcactatg

SEQ ID NO: 82 - DNA, 14 bp, artificial
  Oligonucleotide nucleobase seqeunce
gccataagcactat

SEQ ID NO: 83 - DNA, 14 bp, artificial
  Oligonucleotide nucleobase seqeunce
ggaagaaggggtga

SEQ ID NO: 84 - DNA, 14 bp, artificial
  Oligonucleotide nucleobase seqeunce
gggaagaaggggtg

SEQ ID NO: 85 - DNA, 14 bp, artificial
  Oligonucleotide nucleobase seqeunce
ggggaagaaggggt

SEQ ID NO: 86 - DNA, 14 bp, artificial
  Oligonucleotide nucleobase seqeunce
gggggaagaagggg

SEQ ID NO: 87 - DNA, 14 bp, artificial
  Oligonucleotide nucleobase seqeunce
tgggggaagaaggg

SEQ ID NO: 88 - DNA, 14 bp, artificial
  Oligonucleotide nucleobase seqeunce
gtgggggaagaagg

SEQ ID NO: 89 - DNA, 14 bp, artificial
  Oligonucleotide nucleobase seqeunce
tgtgggggaagaag

SEQ ID NO: 90 - DNA, 14 bp, artificial
  Oligonucleotide nucleobase seqeunce
ctgtgggggaagaa

SEQ ID NO: 91 - DNA, 14 bp, artificial
  Oligonucleotide nucleobase seqeunce
tctgtgggggaaga

SEQ ID NO: 92 - DNA, 14 bp, artificial
  Oligonucleotide nucleobase seqeunce
atctgtgggggaag

SEQ ID NO: 93 - DNA, 14 bp, artificial
  Oligonucleotide nucleobase seqeunce
catctgtgggggaa

SEQ ID NO: 94 - DNA, 14 bp, artificial
  Oligonucleotide nucleobase seqeunce
acatctgtggggga

SEQ ID NO: 95 - DNA, 14 bp, artificial
  Oligonucleotide nucleobase seqeunce
gacatctgtggggg

SEQ ID NO: 96 - DNA, 14 bp, artificial
  Oligonucleotide nucleobase seqeunce
tgacatctgtgggg

SEQ ID NO: 97 - DNA, 14 bp, artificial
  Oligonucleotide nucleobase seqeunce
gtgacatctgtggg

SEQ ID NO: 98 - DNA, 14 bp, artificial
  Oligonucleotide nucleobase seqeunce
gtgacatctgtggg

SEQ ID NO: 99 - DNA, 14 bp, artificial
  Oligonucleotide nucleobase seqeunce
gtgacatctgtggg

SEQ ID NO: 100 - DNA, 14 bp, artificial
  Oligonucleotide nucleobase seqeunce
ggtgacatctgtgg

SEQ ID NO: 101 - DNA, 14 bp, artificial
  Oligonucleotide nucleobase seqeunce
ggtgacatctgtgg

SEQ ID NO: 102 - DNA, 14 bp, artificial
  Oligonucleotide nucleobase seqeunce
ggtgacatctgtgg

SEQ ID NO: 103 - DNA, 14 bp, artificial
  Oligonucleotide nucleobase seqeunce
cggtgacatctgtg

SEQ ID NO: 104 - DNA, 14 bp, artificial
  Oligonucleotide nucleobase seqeunce
cggtgacatctgtg

SEQ ID NO: 105 - DNA, 14 bp, artificial
  Oligonucleotide nucleobase seqeunce
cggtgacatctgtg

SEQ ID NO: 106 - DNA, 14 bp, artificial
  Oligonucleotide nucleobase seqeunce
ccggtgacatctgt

SEQ ID NO: 107 - DNA, 14 bp, artificial
  Oligonucleotide nucleobase seqeunce
ccggtgacatctgt

SEQ ID NO: 108 - DNA, 14 bp, artificial
  Oligonucleotide nucleobase seqeunce
ccggtgacatctgt

SEQ ID NO: 109 - DNA, 14 bp, artificial
  Oligonucleotide nucleobase seqeunce
cccggtgacatctg

SEQ ID NO: 110 - DNA, 14 bp, artificial
  Oligonucleotide nucleobase seqeunce
cccggtgacatctg

SEQ ID NO: 111 - DNA, 14 bp, artificial
  Oligonucleotide nucleobase seqeunce
acccggtgacatct

SEQ ID NO: 112 - DNA, 14 bp, artificial
  Oligonucleotide nucleobase seqeunce
acccggtgacatct

SEQ ID NO: 113 - DNA, 14 bp, artificial
  Oligonucleotide nucleobase seqeunce
acccggtgacatct

SEQ ID NO: 114 - DNA, 14 bp, artificial
  Oligonucleotide nucleobase seqeunce
cacccggtgacatc

SEQ ID NO: 115 - DNA, 14 bp, artificial
  Oligonucleotide nucleobase seqeunce
cacccggtgacatc

SEQ ID NO: 116 - DNA, 14 bp, artificial
  Oligonucleotide nucleobase seqeunce
cacccggtgacatc

SEQ ID NO: 117 - DNA, 14 bp, artificial
  Oligonucleotide nucleobase seqeunce
gcacccggtgacat

SEQ ID NO: 118 - DNA, 14 bp, artificial
  Oligonucleotide nucleobase seqeunce
gcacccggtgacat

SEQ ID NO: 119 - DNA, 14 bp, artificial
  Oligonucleotide nucleobase seqeunce
gcacccggtgacat

SEQ ID NO: 120 - DNA, 14 bp, artificial
  Oligonucleotide nucleobase seqeunce
cgcacccggtgaca

SEQ ID NO: 121 - DNA, 14 bp, artificial
  Oligonucleotide nucleobase seqeunce
gcgcacccggtgac

SEQ ID NO: 122 - DNA, 14 bp, artificial
  Oligonucleotide nucleobase seqeunce
tgcgcacccggtga

SEQ ID NO: 123 - DNA, 14 bp, artificial
  Oligonucleotide nucleobase seqeunce
atgcgcacccggtg

SEQ ID NO: 124 - DNA, 14 bp, artificial
  Oligonucleotide nucleobase seqeunce
aacacgaaaatagg

SEQ ID NO: 125 - DNA, 14 bp, artificial
  Oligonucleotide nucleobase seqeunce
gaacacgaaaatag

SEQ ID NO: 126 - DNA, 14 bp, artificial
  Oligonucleotide nucleobase seqeunce
ggaacacgaaaata

SEQ ID NO: 127 - DNA, 14 bp, artificial
  Oligonucleotide nucleobase seqeunce
gggaacacgaaaat

SEQ ID NO: 128 - DNA, 14 bp, artificial
  Oligonucleotide nucleobase seqeunce
ggggaacacgaaaa

SEQ ID NO: 129 - DNA, 14 bp, artificial
  Oligonucleotide nucleobase seqeunce
tggggaacacgaaa

SEQ ID NO: 130 - DNA, 14 bp, artificial
  Oligonucleotide nucleobase seqeunce
ctggggaacacgaa

SEQ ID NO: 131 - DNA, 14 bp, artificial
  Oligonucleotide nucleobase seqeunce
cctggggaacacga

SEQ ID NO: 132 - DNA, 14 bp, artificial
  Oligonucleotide nucleobase seqeunce
tcctggggaacacg

SEQ ID NO: 133 - DNA, 14 bp, artificial
  Oligonucleotide nucleobase seqeunce
ctcctggggaacac

SEQ ID NO: 134 - DNA, 14 bp, artificial
  Oligonucleotide nucleobase seqeunce
tctcctggggaaca

SEQ ID NO: 135 - DNA, 14 bp, artificial
  Oligonucleotide nucleobase seqeunce
gtctcctggggaac

SEQ ID NO: 136 - DNA, 14 bp, artificial
  Oligonucleotide nucleobase seqeunce
tgtctcctggggaa

SEQ ID NO: 137 - DNA, 14 bp, artificial
  Oligonucleotide nucleobase seqeunce
atgtctcctgggga

SEQ ID NO: 138 - DNA, 14 bp, artificial
  Oligonucleotide nucleobase seqeunce
atgtctcctgggga

SEQ ID NO: 139 - DNA, 14 bp, artificial
  Oligonucleotide nucleobase seqeunce
atgtctcctgggga

SEQ ID NO: 140 - DNA, 14 bp, artificial
  Oligonucleotide nucleobase seqeunce
gatgtctcctgggg

SEQ ID NO: 141 - DNA, 14 bp, artificial
  Oligonucleotide nucleobase seqeunce
gatgtctcctgggg

SEQ ID NO: 142 - DNA, 14 bp, artificial
  Oligonucleotide nucleobase seqeunce
gatgtctcctgggg

SEQ ID NO: 143 - DNA, 14 bp, artificial
  Oligonucleotide nucleobase seqeunce
tgatgtctcctggg

SEQ ID NO: 144 - DNA, 14 bp, artificial
  Oligonucleotide nucleobase seqeunce
tgatgtctcctggg

SEQ ID NO: 145 - DNA, 14 bp, artificial
  Oligonucleotide nucleobase seqeunce
tgatgtctcctggg

SEQ ID NO: 146 - DNA, 14 bp, artificial
  Oligonucleotide nucleobase seqeunce
ttgatgtctcctgg

SEQ ID NO: 147 - DNA, 14 bp, artificial
  Oligonucleotide nucleobase seqeunce
ttgatgtctcctgg

SEQ ID NO: 148 - DNA, 14 bp, artificial
  Oligonucleotide nucleobase seqeunce
ttgatgtctcctgg

SEQ ID NO: 149 - DNA, 14 bp, artificial
  Oligonucleotide nucleobase seqeunce
cttgatgtctcctg

SEQ ID NO: 150 - DNA, 14 bp, artificial
  Oligonucleotide nucleobase seqeunce
cttgatgtctcctg

SEQ ID NO: 151 - DNA, 14 bp, artificial
  Oligonucleotide nucleobase seqeunce
cttgatgtctcctg

SEQ ID NO: 152 - DNA, 14 bp, artificial
  Oligonucleotide nucleobase seqeunce
gcttgatgtctcct

SEQ ID NO: 153 - DNA, 14 bp, artificial
  Oligonucleotide nucleobase seqeunce
gcttgatgtctcct

SEQ ID NO: 154 - DNA, 14 bp, artificial
  Oligonucleotide nucleobase seqeunce
gcttgatgtctcct

SEQ ID NO: 155 - DNA, 14 bp, artificial
  Oligonucleotide nucleobase seqeunce
tgcttgatgtctcc

SEQ ID NO: 156 - DNA, 14 bp, artificial
  Oligonucleotide nucleobase seqeunce
tgcttgatgtctcc

SEQ ID NO: 157 - DNA, 14 bp, artificial
  Oligonucleotide nucleobase seqeunce
tgcttgatgtctcc

SEQ ID NO: 158 - DNA, 14 bp, artificial
  Oligonucleotide nucleobase seqeunce
ctgcttgatgtctc

SEQ ID NO: 159 - DNA, 14 bp, artificial
  Oligonucleotide nucleobase seqeunce
ctgcttgatgtctc

SEQ ID NO: 160 - DNA, 14 bp, artificial
  Oligonucleotide nucleobase seqeunce
ctgcttgatgtctc

SEQ ID NO: 161 - DNA, 14 bp, artificial
  Oligonucleotide nucleobase seqeunce
cctgcttgatgtct

SEQ ID NO: 162 - DNA, 14 bp, artificial
  Oligonucleotide nucleobase seqeunce
tcctgcttgatgtc

SEQ ID NO: 163 - DNA, 14 bp, artificial
  Oligonucleotide nucleobase seqeunce
ttcctgcttgatgt

SEQ ID NO: 164 - DNA, 14 bp, artificial
  Oligonucleotide nucleobase seqeunce
cttcctgcttgatg

SEQ ID NO: 165 - DNA, 1740 bp, homo sapiens
atggctgtgtttaatcagaagtctgtctcggatatgattaaagagtttcgaaaaaattggcgtgctctttgtaactctgagagaactactctatgtggtgcagactccatgctcttggcattgcagctttctatggcggagaacaacaaacagcacagtggagaatttacagtctctctcagtgatgttttattgacatggaaatacttgctccatgagaaattgaacttaccagttgaaaacatggacgtgactgaccattatgaggacgttaggaagatttatgatgatttcttgaagaacagtaatatgttagatctgattgatgtttatcaaaaatgtagggctttgacttctaattgtgaaaattataacacagtatctcctagtcaactactggattttctgtctggcaaacagtatgcagtaggtgatgaaactgatctttctataccaacatcaccaacaagtaaatacaaccgtgataatgaaaaggtgcagctgctagcaaggaaaattatcttttcatatttaaatctgctagtgaattcaaagaatgacctggctgtggcttatattctcaatattcctgatagaggactaggaagagaagccttcactgatttgaaacatgctgctcgagagaaacaaatgtctatctttttggtggccacgtcttttattagaacaatagagcttggagggaaaggatatgcaccaccaccatcagatcctttaaggacacatgtaaagggattgtctaattttattaatttcattgacaaattagatgagattcttggagaaataccaaacccaagcattgcagggggtcaaatactgtcagtgataaagatgcaactgattaaaggccaaaacagcagggatcctttttgcaaagcaatagaggaagttgctcaggatttggatttgaggattaaaaatattatcaattctcaagaaggtgttgtagctcttagcaccactgacatcagtcctgctcggccaaaatctcatgccataaaccatggtactgcatactgtggcagagatactgtgaaagccttattagttcttttggacgaagaagcagctaatgctcctaccaaaaacaaagcagagcttttatatgatgaggaaaacacaatccatcatcatggaacgtctattcttacactttttaggtctcccacacaggtgaataattcgataaaacccctaagagaacgcatctgtgtgtcaatgcaagagaaaaaaattaagatgaagcaaactttaattagatcccaatttgcttgtacttataaagatgactacatgataagcaaggataattggaataatgttaatttagcatcaaagcctttgtgtgttctttacatggaaaatgacctttctgagggtgtaaatccatctgttggaagatcaacaattggaacgagttttggaaatgttcatctggacagaagtaaaaatgaaaaagtatcaagaaaatcaaccagtcagacaggaaataaaagctcaaaaaggaaacaggtggatttggatggtgaaaatattctctgtgataatagaaatgaaccacctcaacataaaaatgctaaaatacctaagaaatcaaatgattcacagaatagattgtacggcaaactagctaaagtagcaaaaagtaataaatgtactgccaaggacaagttgatttctggccaggcaaagttaactcagttttttagactataa

SEQ ID NO: 166 - protein, 237 aa, homo sapiens
MAVFNQKSVSDMIKEFRKNWRALCNSERTTLCGADSMLLALQLSMAENNKQHSGEFTVSLSDVLLTWKYLLHEKLNLPVENMDVTDHYEDVRKIYDDFLKNSNMLDLIDVYQKCRALTSNCENYNTVSPSQLLDFLSGKQYAVGDETDLSIPTSPTSKYNRDNEKVQLLARKIIFSYLNLLVNSKNDLAVAYILNIPDRGLGREAFTDLKHAAREKQMSIFLHCRGSNTVSDKDATD

SEQ ID NO: 167 - DNA, 20 bp, artificial
  probe or primer sequence
gcgatgaccaaattgggaag

SEQ ID NO: 168 - DNA, 18 bp, artificial
  probe or primer sequence
gccatgctctgtctggaa

SEQ ID NO: 169 - DNA, 27 bp, artificial
  probe or primer sequence
caccttggatttaggacatcgaggctc

SEQ ID NO: 170 - DNA, 20 bp, artificial
  probe or primer sequence
ctcggtgctgaggttaactc

SEQ ID NO: 171 - DNA, 21 bp, artificial
  probe or primer sequence
tcagactaaaggtggggagaa

SEQ ID NO: 172 - DNA, 24 bp, artificial
  probe or primer sequence
tcagacctctggaaagctcaccca

SEQ ID NO: 173 - DNA, 19 bp, artificial
  probe or primer sequence
ggaactccattctgcaagg

SEQ ID NO: 174 - DNA, 18 bp, artificial
  probe or primer sequence
cctcagtgtccagtgtca

SEQ ID NO: 175 - DNA, 24 bp, artificial
  probe or primer sequence
tccctgacaaagcaaatggcattc

SEQ ID NO: 176 - DNA, 20 bp, artificial
  probe or primer sequence
ctcggtgctgaggttaactc

SEQ ID NO: 177 - DNA, 21 bp, artificial
  probe or primer sequence
tcagactaaaggtggggagaa

SEQ ID NO: 178 - DNA, 17 bp, artificial
  probe or primer sequence
gggacaacgcagacatc

SEQ ID NO: 179 - DNA, 17 bp, artificial
  probe or primer sequence
gggacaacgcagacatc

SEQ ID NO: 180 - DNA, 22 bp, artificial
  probe or primer sequence
cgctcgctccgatactattatg

SEQ ID NO: 181 - DNA, 21 bp, artificial
  probe or primer sequence
caaactcagccttgagagctg

SEQ ID NO: 182 - DNA, 24 bp, artificial
  probe or primer sequence
catcatgcagaaggtggctggtga

SEQ ID NO: 183 - DNA, 17 bp, artificial
  probe or primer sequence
ctctgcgaccattccca

SEQ ID NO: 184 - DNA, 22 bp, artificial
  probe or primer sequence
gctcagcttgtcgtaattcatg

SEQ ID NO: 185 - DNA, 22 bp, artificial
  probe or primer sequence
cgctcgctccgatactattatg

SEQ ID NO: 186 - DNA, 21 bp, artificial
  probe or primer sequence
caaactcagccttgagagctg

SEQ ID NO: 187 - DNA, 24 bp, artificial
  probe or primer sequence
catcatgcagaaggtggctggtga

SEQ ID NO: 188 - protein, 28 aa, Artificial
  epitope sequence
RTSRLCCCPGIQIVPDRAWRIFQCFFVQ

SEQ ID NO: 189 - protein, 21 aa, artificial
  epitope sequence
KANGIRWLQRQLPAHLGDTGH

SEQ ID NO: 190 - protein, 20 aa, artificial
  epitope sequence
QEQTDFAYDSGYGYEKPLRP

SEQ ID NO: 191 - protein, 27 aa, Artificial
  epitope sequence
GRQALGHPEEPASHELRQAEPLAPILL

SEQ ID NO: 192 - protein, 15 aa, Artificial
  epitope sequence
HCRGSNTVSDKDATD

SEQ ID NO: 193 - DNA, 14 bp, artificial
  Oligonucleotide nucleobase seqeunce
ttagaccagaggga

SEQ ID NO: 194 - DNA, 14 bp, artificial
  Oligonucleotide nucleobase seqeunce
tagaccagagggag

SEQ ID NO: 195 - DNA, 14 bp, artificial
  Oligonucleotide nucleobase seqeunce
agaccagagggaga

SEQ ID NO: 196 - DNA, 14 bp, artificial
  Oligonucleotide nucleobase seqeunce
gaccagagggagag

SEQ ID NO: 197 - DNA, 14 bp, artificial
  Oligonucleotide nucleobase seqeunce
accagagggagaga

SEQ ID NO: 198 - DNA, 14 bp, artificial
  Oligonucleotide nucleobase seqeunce
ccagagggagagag

SEQ ID NO: 199 - DNA, 14 bp, artificial
  Oligonucleotide nucleobase seqeunce
cagagggagagagc

SEQ ID NO: 200 - DNA, 14 bp, artificial
  Oligonucleotide nucleobase seqeunce
agagggagagagca

SEQ ID NO: 201 - DNA, 14 bp, artificial
  Oligonucleotide nucleobase seqeunce
gagggagagagcaa

SEQ ID NO: 202 - DNA, 14 bp, artificial
  Oligonucleotide nucleobase seqeunce
agggagagagcaaa

SEQ ID NO: 203 - DNA, 14 bp, artificial
  Oligonucleotide nucleobase seqeunce
gggagagagcaaac

SEQ ID NO: 204 - DNA, 14 bp, artificial
  Oligonucleotide nucleobase seqeunce
ggagagagcaaact

SEQ ID NO: 205 - DNA, 14 bp, artificial
  Oligonucleotide nucleobase seqeunce
gagagagcaaactg

SEQ ID NO: 206 - DNA, 10 bp, artificial
  Oligonucleotide nucleobase seqeunce
cttacctttc

SEQ ID NO: 207 - DNA, 12 bp, artificial
  Oligonucleotide nucleobase seqeunce
ccttacctttca

SEQ ID NO: 208 - DNA, 16 bp, artificial
  Oligonucleotide nucleobase seqeunce
agttcaaacccttacc

SEQ ID NO: 209 - DNA, 16 bp, artificial
  Oligonucleotide nucleobase seqeunce
ttcaaacccttacctt

SEQ ID NO: 210 - DNA, 16 bp, artificial
  Oligonucleotide nucleobase seqeunce
caaacccttacctttc

SEQ ID NO: 211 - DNA, 16 bp, artificial
  Oligonucleotide nucleobase seqeunce
aacccttacctttcaa

SEQ ID NO: 212 - DNA, 16 bp, artificial
  Oligonucleotide nucleobase seqeunce
cccttacctttcaact

SEQ ID NO: 213 - DNA, 16 bp, artificial
  Oligonucleotide nucleobase seqeunce
cttacctttcaacttg

SEQ ID NO: 214 - DNA, 16 bp, artificial
  Oligonucleotide nucleobase seqeunce
tacctttcaacttgag

SEQ ID NO: 215 - DNA, 16 bp, artificial
  Oligonucleotide nucleobase seqeunce
cctttcaacttgagac

SEQ ID NO: 216 - DNA, 18 bp, artificial
  Oligonucleotide nucleobase seqeunce
agttcaaacccttacctt

SEQ ID NO: 217 - DNA, 18 bp, artificial
  Oligonucleotide nucleobase seqeunce
ttcaaacccttacctttc

SEQ ID NO: 218 - DNA, 18 bp, artificial
  Oligonucleotide nucleobase seqeunce
caaacccttacctttcaa

SEQ ID NO: 219 - DNA, 18 bp, artificial
  Oligonucleotide nucleobase seqeunce
aacccttacctttcaact

SEQ ID NO: 220 - DNA, 18 bp, artificial
  Oligonucleotide nucleobase seqeunce
cccttacctttcaacttg

SEQ ID NO: 221 - DNA, 18 bp, artificial
  Oligonucleotide nucleobase seqeunce
cttacctttcaacttgag

SEQ ID NO: 222 - DNA, 18 bp, artificial
  Oligonucleotide nucleobase seqeunce
tacctttcaacttgagac

SEQ ID NO: 223 - DNA, 20 bp, artificial
  Oligonucleotide nucleobase seqeunce
agttcaaacccttacctttc

SEQ ID NO: 224 - DNA, 20 bp, artificial
  Oligonucleotide nucleobase seqeunce
ttcaaacccttacctttcaa

SEQ ID NO: 225 - DNA, 20 bp, artificial
  Oligonucleotide nucleobase seqeunce
caaacccttacctttcaact

SEQ ID NO: 226 - DNA, 20 bp, artificial
  Oligonucleotide nucleobase seqeunce
aacccttacctttcaacttg

SEQ ID NO: 227 - DNA, 20 bp, artificial
  Oligonucleotide nucleobase seqeunce
cccttacctttcaacttgag

SEQ ID NO: 228 - DNA, 20 bp, artificial
  Oligonucleotide nucleobase seqeunce
cttacctttcaacttgagac

SEQ ID NO: 229 - DNA, 14 bp, artificial
  Oligonucleotide nucleobase seqeunce
gggtcagtttaaac

SEQ ID NO: 230 - DNA, 14 bp, artificial
  Oligonucleotide nucleobase seqeunce
gtcagtttaaaccc

SEQ ID NO: 231 - DNA, 14 bp, artificial
  Oligonucleotide nucleobase seqeunce
cagtttaaacccca

SEQ ID NO: 232 - DNA, 14 bp, artificial
  Oligonucleotide nucleobase seqeunce
gtttaaaccccagt

SEQ ID NO: 233 - DNA, 14 bp, artificial
  Oligonucleotide nucleobase seqeunce
ttaaaccccagttc

SEQ ID NO: 234 - DNA, 14 bp, artificial
  Oligonucleotide nucleobase seqeunce
aaaccccagttcaa

SEQ ID NO: 235 - DNA, 14 bp, artificial
  Oligonucleotide nucleobase seqeunce
accccagttcaaac

SEQ ID NO: 236 - DNA, 14 bp, artificial
  Oligonucleotide nucleobase seqeunce
cccagttcaaaccc

SEQ ID NO: 237 - DNA, 14 bp, artificial
  Oligonucleotide nucleobase seqeunce
cagttcaaaccctt

SEQ ID NO: 238 - DNA, 14 bp, artificial
  Oligonucleotide nucleobase seqeunce
gttcaaacccttac

SEQ ID NO: 239 - DNA, 14 bp, artificial
  Oligonucleotide nucleobase seqeunce
tcaaacccttacct

SEQ ID NO: 240 - DNA, 14 bp, artificial
  Oligonucleotide nucleobase seqeunce
aaacccttaccttt

SEQ ID NO: 241 - DNA, 14 bp, artificial
  Oligonucleotide nucleobase seqeunce
acccttacctttca

SEQ ID NO: 242 - DNA, 14 bp, artificial
  Oligonucleotide nucleobase seqeunce
ccttacctttcaac

SEQ ID NO: 243 - DNA, 14 bp, artificial
  Oligonucleotide nucleobase seqeunce
ttacctttcaactt

SEQ ID NO: 244 - DNA, 14 bp, artificial
  Oligonucleotide nucleobase seqeunce
acctttcaacttga

SEQ ID NO: 245 - DNA, 14 bp, artificial
  Oligonucleotide nucleobase seqeunce
ctttcaacttgaga

SEQ ID NO: 246 - DNA, 14 bp, artificial
  Oligonucleotide nucleobase seqeunce
ttcaacttgagacc

SEQ ID NO: 247 - DNA, 14 bp, artificial
  Oligonucleotide nucleobase seqeunce
caacttgagacccc

SEQ ID NO: 248 - DNA, 14 bp, artificial
  Oligonucleotide nucleobase seqeunce
acttgagacccctg

SEQ ID NO: 249 - DNA, 14 bp, artificial
  Oligonucleotide nucleobase seqeunce
ttgagacccctgtc

SEQ ID NO: 250 - DNA, 14 bp, artificial
  Oligonucleotide nucleobase seqeunce
gagacccctgtctg

SEQ ID NO: 251 - DNA, 14 bp, artificial
  Oligonucleotide nucleobase seqeunce
gacccctgtctgcc

SEQ ID NO: 252 - DNA, 14 bp, artificial
  Oligonucleotide nucleobase seqeunce
cccctgtctgcccc

SEQ ID NO: 253 - DNA, 14 bp, artificial
  Oligonucleotide nucleobase seqeunce
cctgtctgccccaa

SEQ ID NO: 254 - DNA, 14 bp, artificial
  Oligonucleotide nucleobase seqeunce
tgtctgccccaagc

SEQ ID NO: 255 - DNA, 14 bp, artificial
  Oligonucleotide nucleobase seqeunce
tctgccccaagctt

SEQ ID NO: 256 - DNA, 14 bp, artificial
  Oligonucleotide nucleobase seqeunce
tgccccaagctttt

SEQ ID NO: 257 - DNA, 14 bp, artificial
  Oligonucleotide nucleobase seqeunce
ccccaagcttttcc

SEQ ID NO: 258 - DNA, 14 bp, artificial
  Oligonucleotide nucleobase seqeunce
ccaagcttttccag

SEQ ID NO: 259 - DNA, 14 bp, artificial
  Oligonucleotide nucleobase seqeunce
aagcttttccagca

SEQ ID NO: 260 - DNA, 14 bp, artificial
  Oligonucleotide nucleobase seqeunce
gcttttccagcact

SEQ ID NO: 261 - DNA, 14 bp, artificial
  Oligonucleotide nucleobase seqeunce
ttttccagcactct

SEQ ID NO: 262 - DNA, 14 bp, artificial
  Oligonucleotide nucleobase seqeunce
ttccagcactctgg

SEQ ID NO: 263 - DNA, 14 bp, artificial
  Oligonucleotide nucleobase seqeunce
ccagcactctggtc

SEQ ID NO: 264 - DNA, 14 bp, artificial
  Oligonucleotide nucleobase seqeunce
agcactctggtcca

SEQ ID NO: 265 - DNA, 14 bp, artificial
  Oligonucleotide nucleobase seqeunce
cactctggtccatg

SEQ ID NO: 266 - DNA, 14 bp, artificial
  Oligonucleotide nucleobase seqeunce
ctctggtccatggg

SEQ ID NO: 267 - DNA, 14 bp, artificial
  Oligonucleotide nucleobase seqeunce
ctggtccatgggcc

SEQ ID NO: 268 - DNA, 14 bp, artificial
  Oligonucleotide nucleobase seqeunce
ggtccatgggcctc

SEQ ID NO: 269 - DNA, 14 bp, artificial
  Oligonucleotide nucleobase seqeunce
tccatgggcctctg

SEQ ID NO: 270 - DNA, 14 bp, artificial
  Oligonucleotide nucleobase seqeunce
catgggcctctgga

SEQ ID NO: 271 - DNA, 14 bp, artificial
  Oligonucleotide nucleobase seqeunce
tgggcctctggaga

SEQ ID NO: 272 - DNA, 14 bp, artificial
  Oligonucleotide nucleobase seqeunce
ggcctctggagacg

SEQ ID NO: 273 - DNA, 14 bp, artificial
  Oligonucleotide nucleobase seqeunce
cctctggagacgta

SEQ ID NO: 274 - DNA, 14 bp, artificial
  Oligonucleotide nucleobase seqeunce
tctggagacgtatc

SEQ ID NO: 275 - DNA, 50 bp, homo sapiens
gacagcggcgactgcggcggccgcgggagggcatcccgttggggatcctt

SEQ ID NO: 276 - DNA, 42 bp, homo sapiens
ccgcacactgaagagtacgtcttcgggtctacccctaatcac

SEQ ID NO: 277 - DNA, 400 bp, homo sapiens
gtaagtctcgcgtctgccctacctgccctgtcgcatttttaagtggtgattcaggctgtggcttttgagggtgttttgcactgtaatacctcggtcgtagcaaagtgccgtgggaccgaagtattatggtgcagtgacttgacgctcgagcctggttgggcctgggggctgtaggcggagttcacagtatctagcgatggaccaaaggggagggaaatatgattgcgcagaaattgtattattttgttttcctaaatttaattaaaagcaatcttggtaaatatgaaatgacctctcattcactatatgtttttctctatctgtgcttgcctatacatgttaaatgttaatttcaggttaacataagatactttaactttgtattctgtcctactttaag

SEQ ID NO: 278 - DNA, 156 bp, homo sapiens
ataatggctgtgtttaatcagaagtctgtctcggatatgattaaagagtttcgaaaaaattggcgtgctctttgtaactctgagagaactactctatgtggtgcagactccatgctcttggcattgcagctttctatggcggagaacaacaaacag

SEQ ID NO: 279 - DNA, 400 bp, homo sapiens
gttggtaaactatatttgggttaacttgtttttttaaagcaaaatttcattgctgcccatttgaataaacttaagcttaagaatattaatttctttacattatgtgcccttctgtgataatacaccattttcacaaatgcctctgtgtatctgtgtcagaatgttatgatagaatttgaaatgaattatttcaaaattaataaaatcatgaaaacctttttcttttatatctggtttccaaaattgtctcatagttcaacttgggtgttctcttcaaacccacaaaaattgaatgtagactttaccaaaatttaatttgtatttcttatctcagttataagttattgaatattctggtacccaactttattttttttttttttggcattatctttttcag

SEQ ID NO: 280 - DNA, 234 bp, homo sapiens
cacagtggagaatttacagtctctctcagtgatgttttattgacatggaaatacttgctccatgagaaattgaacttaccagttgaaaacatggacgtgactgaccattatgaggacgttaggaagatttatgatgatttcttgaagaacagtaatatgttagatctgattgatgtttatcaaaaatgtagggctttgacttctaattgtgaaaattataacacagtatctcct

SEQ ID NO: 281 - DNA, 400 bp, homo sapiens
gtaagtattttttaaacaattctattttaatcaaattaaaatttagctctatttattttgaattatagtatcaccagttatagtaacttggtaatgtttttgctttgtgaatcatgttaaaaatgggaaaatttagctacctgaaagctacttattacttgaaagaaagattttgtggtaaattttcttggacttgtgttgtctttatttgtgtagcacacaggaagaacccatgggcaattacattatgaagtttaattatatgtattcttaatgattgctaacaactgtcaggtaatactctcaactgaaacatcaatgacactgagtagagctttatgtgaagtattttttatagtcagcatagcattttattagtaatactctatgttttctacag

SEQ ID NO: 282 - DNA, 108 bp, homo sapiens
agtcaactactggattttctgtctggcaaacagtatgcagtaggtgatgaaactgatctttctataccaacatcaccaacaagtaaatacaaccgtgataatgaaaag

SEQ ID NO: 283 - DNA, 400 bp, homo sapiens
gtactgataaactgtgagtaaattaaagcagactataatccctttcaaatcactgaatttggtggtaccttaaagttactaatgctttgtaaaaatttttttaacaaatctttagtatgtttaaaactgtattcgcaaagaataatacattggaggctggtgagaatgtgataaaagagtctttatggtttggggccaagtttggtagtcagtcaagaatttggggagagatggagtttattgattgcccaacctgtttttatatggcagttttatttttctaaagagttaagtgaggtattgtgattgaatgattacatacatttttactgcttatgaaaaagattatggatttagaagaactgcaataaagaaattaactgaatattttattgcacag

SEQ ID NO: 284 - DNA, 171 bp, homo sapiens
gtgcagctgctagcaaggaaaattatcttttcatatttaaatctgctagtgaattcaaagaatgacctggctgtggcttatattctcaatattcctgatagaggactaggaagagaagccttcactgatttgaaacatgctgctcgagagaaacaaatgtctatctttttg

SEQ ID NO: 285 - DNA, 400 bp, homo sapiens
gtatggttgtgtgacatgttcaaaattaagactccttgcacagtggatccatgtatcctaatatgcttgtaactaggatgaaacaacttttaagtggttaatttatgttctacctctgtatttgaaaatccatggtattttaattttttaccatatcgttcaagtttggtaaaggagaatttctctaaagcagtgtttccaccacacttgctagcatgaattctgcatggcatttgtttctttggttatttacttctgactcaaagtgtgtcatgagccataaaatgtgcttcacttttaactgtatacccagaattatatagatttacttagctttatgaagtaaattacagtttaataaatcactggacataacttatccgtttgttttaattcatag

SEQ ID NO: 286 - DNA, 155 bp, homo sapiens
gtggccacgtcttttattagaacaatagagcttggagggaaaggatatgcaccaccaccatcagatcctttaaggacacatgtaaagggattgtctaattttattaatttcattgacaaattagatgagattcttggagaaataccaaacccaag

SEQ ID NO: 287 - DNA, 400 bp, homo sapiens
gtaatgacttttcatatattacaaatatgtttttaatctatctttaaaactttacagaattgggtagaaataagggatattgaaatgaccaagcatcagaccaaacggtgaggcgaagaaacaaaatctagatgaataaattataatcacatgaccttacctcctaaatttctgcctgtgattctgagttttggccaaagaaattacttgcttttagacaaacttttaaattttgtttctaatggtccttatctttttcttaaatgctttaactgtttgatatataagatcaaagctatattctttgaaaagtgttaactctgaaaattatattttcaagtctataagcatattataatttgcaatactgctatagaggcaatctaatttctattttcag

SEQ ID NO: 288 - DNA, 184 bp, homo sapiens
cattgcagggggtcaaatactgtcagtgataaagatgcaactgattaaaggccaaaacagcagggatcctttttgcaaagcaatagaggaagttgctcaggatttggatttgaggattaaaaatattatcaattctcaagaaggtgttgtagctcttagcaccactgacatcagtcctgctcgg

SEQ ID NO: 289 - DNA, 400 bp, homo sapiens
gtaatgagctttttatttttcattatccttttatacaaatcattatctcttctatttattgtagaaatggtttattattgctacttaagtcagaagagtgtgtttcctgtgttctaaaatgatattgaaaatgtaagtttctcaataggattatgtaggattacatgttcaacttgggtaaattatcacatatttgggccgtgcttttggaattggtaaagttgattatagcatggcttaaggaaaaatggtcacatgcctcacagccacttcttatagctgatgttttattactttaacaatagatgaaagaggccagtgccacagggaatttaaaagtatggccattcacccagctggggtgattattacatttacctaaaattattttttgtctcag

SEQ ID NO: 290 - DNA, 179 bp, homo sapiens
ccaaaatctcatgccataaaccatggtactgcatactgtggcagagatactgtgaaagccttattagttcttttggacgaagaagcagctaatgctcctaccaaaaacaaagcagagcttttatatgatgaggaaaacacaatccatcatcatggaacgtctattcttacactttttag

SEQ ID NO: 291 - DNA, 400 bp, homo sapiens
gtaagttatgtggaagttatatgtgttataaatgttaactctagagaattataaaagaaatgtatgcttatggtaggaaacttagaaactacagaaattgtgaagaaagaaaataaaagttaattaaagggagattgaacatttgaattcaaagaggttatatataaaagaagtaatattttatgagagtacagcctgaggttcggtggattgggtgtattaaatgcatttttgacttaatggtatttttaatttatgatgagtttatctgggaggtaaccccatcataagttgaggaaaaactagattgtattgggaattaagtttcaacgtgaatttggagggaaacaaacattccaaccatagcaataaatttttgccttttttttttttgacatag

SEQ ID NO: 292 - DNA, 79 bp, homo sapiens
gtctcccacacaggtgaataattcgataaaacccctaagagaacgcatctgtgtgtcaatgcaagagaaaaaaattaag

SEQ ID NO: 293 - DNA, 400 bp, homo sapiens
gtacaatttaatgcatgccatgaaaatagcaaacactgttttatttttagaagaaataaatacatgaagctgagctgggtattgtaaatattgtaaacattgtacatgacacagtaggaattaaatggaaaatgagttatgacagttgacgtacagagactttccttttatacagtcttgactaatctgtttctggtttgaagaacaaaaatgctagccataacaatgaatgagtttgcaaactatgaaatataaaaggttagagagaggttctcaatgagtaatagaatttctaagggaaatttttataccttgattactatagagtgtgtgtctatctagatgaagaaataaaataatgaataaatttgcatattttcttctttctttctgttactag

SEQ ID NO: 294 - DNA, 136 bp, homo sapiens
atgaagcaaactttaattagatcccaatttgcttgtacttataaagatgactacatgataagcaaggataattggaataatgttaatttagcatcaaagcctttgtgtgttctttacatggaaaatgacctttctg

SEQ ID NO: 295 - DNA, 400 bp, homo sapiens
gtaattgaccttatttgtgcaattaaataacaatttaattctagtctactaattagttttaacttaagtaaaattaggttatttttgcaaagtactatgaaatgtaaagggtaatatttttaaagacctcagtttatttaaaaagttaaatctaagtacaaatttatcgataggaataggacacagtttgatttaactaataactcagaggtgctatgataaaatgaaaagtaataggatagtaatgtagctatatttgtcaatcatattatttatagcattattttactttaaaaatttgaatattcgttagccatttttaaagtttattgtaaagttctcgtaaatattaatactcaatatcatgtaattcctttcccctttttatcttgcataacag

SEQ ID NO: 296 - DNA, 1570 bp, homo sapiens
agggtgtaaatccatctgttggaagatcaacaattggaacgagttttggaaatgttcatctggacagaagtaaaaatgaaaaagtatcaagaaaatcaaccagtcagacaggaaataaaagctcaaaaaggaaacaggtggatttggatggtgaaaatattctctgtgataatagaaatgaaccacctcaacataaaaatgctaaaatacctaagaaatcaaatgattcacagaatagattgtacggcaaactagctaaagtagcaaaaagtaataaatgtactgccaaggacaagttgatttctggccaggcaaagttaactcagttttttagactataaatttgtgtcttatatgctttaggtttatgtatctataaaccattcaccaaagacatgcttaatttttaagagatcaaggtgtaaattatgatgatttattattttggtctacagtgtatgtaaggttagtatgttaagcattgtttaaaaatactagtaagtcataattatgcagaattttcacaaagtttaatgcacagagaaagcatatcatttcagttactgatacatcttaacactactttcttttaaaacagacatttaacatacacaagttatagtagcagtatgggcttctcctcccattggcaattaaatgcttttattttcttctgaaaagatgatgtggaccaacaggtatcagacttgccaacaaggtcggtagactcttcccagcatacatctgagcactgaaggaagaagaaagtttaaattgtttaaaggactataattatcacacaaaatttattaagaaaaaaagaatggatctagtataactaattctgagtaaaccaaaatgataataattaattgttgctatttaatcccacatttttggcaggtgtaattgagccatggtcttatttgattttgttatgattgcatccaaattcactttaactcagagttctgtttaatggtggtaggatgtaagaattgaattttgaaaagactactcactgtcaaaatctctccttcctataggaaatttagctgagttttcttcatccccaatttctctcttttcttgtgttgattcagtattctgaactccattctcagctgggaaagctacagatccttttagtgcaagataaggttttatagccagattcagtggcagaccatgatttaagaaattatgtttggagcctgtgttctgtaaagagaaggttgatttggtttttagctatcgtattcggagtggaactataatacaattgtataatattcttgttgatcaattcaaagttactctgcactgtttttgactttttaaaaataccttagatgcaaatttataggagaaaaaacactttcagataagaggtgtttgctgggatggaagaactacctggcatgtaagaaatatcgtcagtcgtcctaatgcatattgtgactgtttgcatatacttctgtttataaaagtatcagttttacttttcagaggatttgtaagaatcatttaaattttcattgaaataaacgacaagtcacattgccactta

SEQ ID NO: 297 - DNA, 216 bp, homo sapiens
attacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacag

SEQ ID NO: 298 - DNA, 21 bp, Artificial
  probe or primer sequence
ttgtccctgagagtcgccagg

(19) United States
(12) Patent Application Publication
(10) Pub. No.: US 2022/0160870 A1
(43) Pub. Date: May 26, 2022

(54) NEOANTIGEN ENGINEERING USING SPLICE MODULATING COMPOUNDS

(71) Applicant: Roche Innovation Center Copenhagen A/S, Horsholm (DK)

(72) Inventors: Troels Koch, København S (DK); Kamille Dumong Erichsen, Horsholm (DK); Mads Jensen, Farum (DK); Jonas Vikesaa, Fredensborg (DK); Lars Joenson, Viby Sjaelland (DK); Klaus Jensen, Copenhagen (DK); Gianluigi Lichinchi, Horsholm (DK)

(21) Appl. No.: 17/271,491

(22) PCT Filed: Aug. 28, 2019
(86) PCT No.: PCT/EP2019/072898

§ 371 Date: Feb. 25, 2021

(30) Foreign Application Priority Data
Aug. 28, 2018 (EP) 18191271.8
Sep. 18, 2018 (EP) 18194983.5
Jun. 4, 2019 (EP) 19178168.1

(51) Int. Cl. A61K 39/395; A61K 48/00; C12N 15/113; A61K 39/00; A61P 35/00; C07K 14/47

(52) U.S. Cl. CPC A61K 39/39583; A61K 48/0058; C12N 15/113; A61K 39/0011; A61K 38/00; C07K 14/47; C12N 15/1135; C12N 2310/11; A61P 35/00

(57) ABSTRACT

The invention relates to the field of immunotherapy and vaccine treatment of diseased cells via enhancing the immune response to the diseased cells. In the context of the present invention this is done by engineering neo-antigens in cells via oligonucleotide mediated production of aberrant RNA transcripts which, when transcribed in the cell, result in the generation or increased expression of aberrant polypeptides. Extracellular display of these polypeptides, of peptide fragments derived provides antigen epitopes (neoantigen) for detection by the immune system.

### FIELD OF INVENTION

The invention relates to the field of immunotherapy and vaccine treatment of diseased cells via enhancing the immune response to the diseased cells. In the context of the present invention this is done by engineering neo-antigens in cells via modulating RNA transcripts, for example via splice modulation or RNA editing, e.g. via oligonucleotide mediated production of aberrant RNA transcripts which, when transcribed in the cell, result in the generation or increased expression of aberrant polypeptides. Extracellular display of these polypeptides, of peptide fragments derived provides antigen epitopes (neoantigen) for detection by the immune system. The methods of the invention may be combined with the use of vaccines or immunotherapy agents to stimulate the immune system to recognize the neoantigen.

### BACKGROUND

RNA modifying oligonucleotides, such as splice modulating antisense oligonucleotides are amongst the first antisense compounds which have been approved, for the treatment of genetic diseases, such as Duchenne muscular dystrophy and spinal muscular atrophy. Rather than degrade the RNA target, RNA modifying oligonucleotides modify RNA transcripts to product a transcript variant which may encode for an altered polypeptide as compared to the unmodified RNA transcript.

RNA modifying oligonucleotide include splice modifying oligonucleotides which alter the splicing of the target pre-mRNA, or RNA editing oligonucleotides, which can introduce insertions, deletions of substitutions (such as A to G substitutions), and can therefore be used for example to modify or insert start and stop codons to produce transcript variants which encode for an altered polypeptide.

Recently, antisense oligonucleotides have been developed that recruit endogenous human ADARs (adenosine deaminase acting on RNA) to edit endogenous transcripts in a simple and programmable way (Merkle et al., Nat Biotechnol. 2019 February; 37(2):133-138). Several important biological processes are under the control of alternative splicing, as many, as 95% of all human multi-exon genes undergo alternative splicing that generate proteins of potential different functions (Matlin et al., Nat Rev Mol Cell Biol. 2005; 6(5):386-98; et al., Nat Genet. 2008; 40(12):1413-5). For example, the isoforms of BCLX are anti-apoptotic and pro-apoptotic, respectively (Revil et al., Mol Cell Biol. 2007; 27(24):8431-41). Therefore, it is crucial for cells to tightly regulate and control the activity of alternative splicing. However, as previously reported, antisense oligonucleotides can be used to elegantly perturb splicing to create alternative or de novo mRNA isoforms with desired functions, see for example Graziewicz et al., Mol Ther. 2008; 16(7):1316-22.

Vormehr et al., doi:10.1038/nature14426 (abstract) reports that mutant MHC class II epitopes drive therapeutic immune responses to cancer. Kahles et al., Cancer Cell 34, 1-14, 2018 refers to a comprehensive analysis of alternative splicing across tumors from 8,705 patients.

Sahin and Tureci, Science 359, 1355-1360 (2018) reviews the art of personalized cancer vaccines for cancer immunotherapy: “Cancer is characterized by an accumulation of genetic alterations. Somatic mutations can generate cancer-specific neoepitopes that are recognized by autologous T-cells as foreign and constitute ideal cancer vaccine targets. Every tumor has its own unique composition of mutations, with only a small fraction shared between patients. Technological advances in genomics, data science, and cancer immunotherapy now enable the rapid mapping of the mutations within a genome, rational selection of vaccine targets, and on-demand production of a therapy customized to a patient's individual tumor.”

Sahin et al., Nature volume 547, pages 222-226 (13 Jul. 2017) reports that personalized RNA mutanome vaccines mobilize poly-specific therapeutic immunity against cancer Neon Therapeutics is developing neoantigen therapies based on unique cancer epitope peptides, see for example Pa et al., Nature. 2017 Jul. 13; 547(7662):217-221. Biontech is exploiting mRNA technologies for cancer immunotherapies.

These approaches require the characterization of a patient's tumor gene expression and epitope display prior to the selection or the creation of a vaccine or immunotherapy therapeutic. This is costly and time consuming, and either requires the development of a patient specific therapy, or limits the possibility of treating some cancers based on the profile of cancers to which there is already an approved therapeutic.

There is therefore a need for a therapeutic treatments using immunotherapy or cancer vaccine which are independent of the endogenous epitope profile of the tumor, and which may therefore be used to treat a broader patient group than current cancer vaccine or immunotherapy treatments. This is achieved in the methods of the present invention by the engineering of a peptide epitope in the cancer or tumor cell (an engineered epitope).

The invention provides a new use of splice modulating or RNA editing oligonucleotides for molecular targeted immunomodulation with the aim to make cancer therapy more efficient and wider application, via a novel concept of modulating splicing events in RNA transcripts or editing RNA transcripts (producing aberrant transcripts) to enable ‘genetic tagging’ of tumor cells by neo-antigens encoded by aberrant transcripts, in order to trigger of enhance the immune system and initiate an anti-tumor response.

There are numerous ways in which splice modulating or RNA editing agents can induce the expression of aberrant polypeptides, which are presented on the cell surface (e.g. via the major histocompatibility complex mechanism) or via the targeting mRNAs which encode a polypeptide membrane binding/trans-membrane domain. Alternatively, the aberrant polypeptide may be secreted.

Even if the efficacy of the splice modulation or RNA editing is low due to the inherent biological signal amplification of T-cell responses, it is likely to result in a durable and potentially strong anti-tumor effect. By selecting target RNAs which are expressed in target cells, such as RNA transcripts whose expression is de-regulated in cancer cells (RNA transcripts which are over-expressed as compared to non-cancer cells) the invention can be used to preferentially or selectively target the immune system to attack the target cell.

### STATEMENT OF THE INVENTION

The invention provides for a method for engineering a peptide epitope in a cell, said method comprising administration of an effective amount of a RNA modifying oligonucleotide to the cell, wherein the RNA modifying targets a target RNA to modulate the RNA to produce an aberrant RNA transcript encoding an aberrant polypeptide containing the peptide epitope.

By way of example, the RNA modifying oligonucleotide may be a splice modulating oligonucleotide or an RNA editing oligonucleotide.

The invention provides for a method for engineering a peptide epitope in a cell, said method comprising administration of an effective amount of an RNA modifying oligonucleotide to the cell, wherein the RNA modifying oligonucleotide targets a target RNA to modulate the coding sequence of the target RNA to produce an aberrant RNA transcript encoding an aberrant polypeptide containing the peptide epitope.

In some embodiments, the RNA modifying oligonucleotide is an RNA editing oligonucleotide, which is capable of introducing a nucleobase insertion, a deletion or a substitution in the target RNA, thereby altering the coding sequence of the target RNA.

In some embodiments, the RNA modifying oligonucleotide is capable of introducing a single base substitution in the target RNA, such as an adenosine to inosine base substitution.

In some embodiments, the RNA modifying oligonucleotide is capable of recruiting a human ADAR.

The invention provides for a method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope.

The invention provides for a method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope, wherein the splice modulating oligonucleotide modulates the of splicing of the target RNA, such as a pre-mRNA, to produce an aberrant RNA transcript introduced by the modulated splicing event, wherein the aberrant RNA (such as mRNA) transcript encodes an internal polypeptide deletion, to produce an aberrant polypeptide comprising an aberrant peptide sequence at the modulated splicing event (e.g. by skipping one or more exons), to produce the peptide epitope.

The invention provides for a method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope; wherein the splice modulating oligonucleotide modulates the splicing of the target RNA, such as a pre-mRNA, to produce an aberrant RNA transcript (such as a mRNA) introduced by the modulated splicing event, wherein the aberrant RNA transcript encodes one or more codons from an intronic region of the target RNA, to produce an aberrant polypeptide comprising an aberrant peptide sequence which includes at least one or more peptide(s) encoded by the one or more codons originating from the intronic region, to produce the peptide epitope.

The invention provides for a method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope; wherein the splice modulating oligonucleotide modulates the of splicing of the target RNA, such as a pre-mRNA, to produce an aberrant RNA transcript comprising a codon frame shift introduced by the modulated splicing event, wherein the aberrant RNA transcript produces a polypeptide with a C-terminal region of at least 1 amino acid, which is transcribed from the region of the aberrant RNA transcript at or 3′ to the codon frame shift.

The invention provides a method for engineering a peptide epitope, which may be referred to herein as a neoantigen peptide, in or a cell, said method comprising administration of an effective amount of a splice modulating oligonucleotide to the cell, wherein the splice modulating oligonucleotide targets an RNA splice event (splice site or splice regulatory region) to modulate the splicing of the RNA (referred to herein as the target RNA) at the splice site to produce an aberrant RNA transcript encoding an aberrant polypeptide containing the peptide epitope.

The method(s) may be an in vitro method or an in vivo method. For in vivo use, the expression of the peptide epitope (neo-antigen) may be used to induce or enhance an immune response.

The invention provides for a method of immune modulating a target cell in a subject, said method comprising the steps of:

a. Vaccinate the subject with an agent comprising a peptide epitope, or encoding peptide epitope;
b. Administer a splice modulating oligonucleotide to the subject, wherein the splice modulating oligonucleotide targets a target RNA in a target cell in the subject, and modulates the splicing of the target RNA to produce an aberrant RNA transcript encoding an aberrant polypeptide containing the peptide epitope;
to trigger or enhance the immune response by the subject to the peptide epitope, such as a target cell expressing the peptide epitope;
wherein step a. and step b: may be in the order of step a. and then step b., or step b. and then step a., or step a. and step b. are performed simultaneously.

The invention provides for a method of immune modulating a target cell in a subject, said method comprising the steps of:

a. administer a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing a peptide epitope;
b. administer an antibody to the subject, wherein the antibody is specific for the peptide epitope
to trigger or enhance the immune response by the subject to the peptide epitope, such as a target cell expressing the peptide epitope; wherein step a. and step b: may be in the order of step a. and then step b., or step b. and then step a., or step a. and step b. are performed simultaneously.

The invention provides for a method of immune modulating a target cell in a subject, said method comprising the step of administering a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing the peptide epitope;

to trigger or enhance the immune response by the subject to the peptide epitope, such as a target cell expressing the peptide epitope.

The invention provides for a method of immunotherapy treatment of a disease in a subject, said method comprising the steps of

a. administer a splice modulating oligonucleotide to the subject, wherein the splice modulating oligonucleotide targets a target RNA in the target cell in the subject, and modulates the splicing of the target RNA to produce an aberrant RNA transcript encoding an aberrant polypeptide containing a peptide epitope;
b. administer an immunotherapy antibody to the subject, wherein the immunotherapy antibody is specific for the peptide epitope;
to trigger or enhance the immune response by the subject to the peptide epitope, such as the peptide epitope expressed by the target cell; wherein step a. and step b: may be in the order of step a. and then step b., or step b. and then step a., or step a. and step b. are performed simultaneously.

The invention provides method of immune modulating a target cell in a subject, said method comprising the administration of a splice modulating oligonucleotide to the subject, wherein the splice modulating oligonucleotide targets a RNA splice site (such as intron/exon boundaries or other RNA splice regulatory regions) in the target cell in the subject, and modulates the splicing of the RNA at the splice site to produce an aberrant mRNA transcript encoding an aberrant polypeptide containing the peptide epitope; wherein the aberrant epitope is immunogenic to the subject; to trigger or enhance the immune response by the subject to the target cell.

The invention provides a method of immune modulating a target cell in a subject, said method comprising the steps of:

a. Vaccinate the subject with an agent comprising a peptide epitope, or encoding a peptide epitope;
b. Administer a splice modulating oligonucleotide to the subject, wherein the splice modulating oligonucleotide targets a RNA splice site (Including RNA splice regulatory regions) in the target cell in the subject, and modulates the splicing of the RNA at the splice site to produce an aberrant mRNA transcript encoding an aberrant polypeptide containing the peptide epitope;
to trigger or enhance the immune response by the subject to the target cell; wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response.

In some embodiments, between steps a and b or b and a, an optionally waiting step c. may be employed, to e.g. allow the subject to develop an adaptive immune response to the antigen peptide (order of steps a, c, b), or to allow the expression of the epitope peptide on the target cell (order of steps b, c, a).

The invention provides for a method of immune modulating a target cell in a subject, said method comprising the steps of:
a. administer a splice modulating oligonucleotide to the subject, wherein the splice modulating oligonucleotide targets a RNA splice site in the target cell in the subject, and modulates the splicing of the RNA at the splice site to produce an aberrant RNA transcript encoding an aberrant polypeptide containing the peptide epitope;
b. administer an antibody to the subject, wherein the antibody is specific for peptide epitope
   to trigger or enhance the immune response by the subject to the target cell, wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response particularly when the antibody is administered in step b.

A waiting step c. may be performed between steps a and b, for example to allow for the expression of the peptide epitope in the target cell (order of steps a, b, c).

The invention provides for the use of a splice-modulating oligonucleotide for the production of a peptide epitope in a cell.

The invention provides for the use of a splice modulating oligonucleotide in the immunotherapy treatment, e.g. of cancer, wherein the splice modulating oligonucleotide targets a RNA to produce an aberrant RNA transcript encoding an aberrant polypeptide containing the peptide epitope, in the cell e.g. in the cancer cell; wherein the immunotherapy treatment comprises the administration of an therapeutic antibody which recognizes the peptide epitope to the subject.

In some embodiments, more than 1 splice modulating oligonucleotide, such as 2 splice modulating oligonucleotides, may be used to provide the effective modulation of splicing to induce the synthesis of the aberrant polypeptide. Such use of multiple splice modulating oligonucleotides allows the targeting of more than splice regulator regions, which can provide enhanced splice modulating effectiveness. Multiple splice modulating oligonucleotides may be delivered as a single oligonucleotide “poly-oligo” construct—see for example WO2015/113922). The more than 1 splice modulating oligonucleotides may target different splicing events (in the same of or different RNA targets), and may therefore result in the production of more than 1 aberrant polypeptide. Use of multiple splice modulating oligonucleotides can thereby induce the synthesis of multiple neoepitopes, which may be advantageous in eliciting an immune response in the subject.

The invention provides for the use of a splice modulating oligonucleotide in the cancer vaccine therapy, wherein the splice modulating oligonucleotide targets a RNA to produce an aberrant RNA transcript encoding an aberrant polypeptide containing the peptide epitope in a cancer cell wherein the vaccine therapy results in the generation of, or enhances the immune response, by the subject to the peptide epitope.

The invention provides for an antisense oligonucleotide capable of modulating the splicing of CEMIP pre-mRNA, wherein the antisense oligonucleotide comprises a contiguous nucleotide sequence of at least 10 nucleotides, such as at least 12 nucleotides, which have 100% identity with a sequence selected from SEQ ID NO 1-41.

The invention provides for an antisense oligonucleotide capable of modulating the splicing of CEMIP pre-mRNA, wherein the antisense oligonucleotide comprises a contiguous nucleotide sequence of at least 10 nucleotides, such as at least 12 nucleotides, which have 100% identity with a sequence selected from SEQ ID NO 42-82.

The invention provides for an antisense oligonucleotide capable of modulating the splicing of CEMIP pre-mRNA, wherein the antisense oligonucleotide comprises a contiguous nucleotide sequence of at least 10 nucleotides, such as at least 12 nucleotides, which have 100% identity with a sequence selected from SEQ ID NO 193-274.

The invention provides for an antisense oligonucleotide capable of modulating the splicing of CEMIP pre-mRNA, wherein the antisense oligonucleotide is selected from compound O1-O41 The invention provides for an antisense oligonucleotide capable of modulating the splicing of CEMIP pre-mRNA, wherein the antisense oligonucleotide is selected from compound O42-O82.

The invention provides for an antisense oligonucleotide capable of modulating the splicing of CEMIP pre-mRNA, wherein the antisense oligonucleotide is selected from compound O65-O246.

The invention provides for an antisense oligonucleotide capable of modulating the splicing of ETV4 pre-mRNA, wherein the antisense oligonucleotide comprises a contiguous nucleotide sequence of at least 10 nucleotides, such as at least 12 nucleotides, which have 100% identity with a sequence selected from SEQ ID NO 83-123.

The invention provides for an antisense oligonucleotide capable of modulating the splicing of ETV4 pre-mRNA, wherein the antisense oligonucleotide is selected from compound 083-0123.

The invention provides for an antisense oligonucleotide capable of modulating the splicing of ETV4 pre-mRNA, wherein the antisense oligonucleotide comprises a contiguous nucleotide sequence of at least 10 nucleotides, such as at least 12 nucleotides, which have 100% identity with a sequence selected from SEQ ID NO 124-164.

The invention provides for an antisense oligonucleotide capable of modulating the splicing of ETV4 pre-mRNA, wherein the antisense oligonucleotide is selected from compound O124-O164.

The antisense oligonucleotides of the invention may be used in the methods and uses of the invention.

The invention provides for a vaccine or immunotherapy agent which comprises the peptide epitope, such as a peptide epitope selected from the group SEQ ID NO 188, 189, 190, 191 & 192.

The invention provides for a polypeptide which is or comprises the peptide, such as a peptide selected from the group SEQ ID NO 188, 189, 190, 191 and 192.

The invention provides for a polypeptide which is or comprises the peptide, such as a peptide selected from the group SEQ ID NO 188, 189, 190, 191 and 192, for use in medicine, such as for use as a vaccine or immunotherapy agent.

The peptide epitope, polypeptide, vaccine or immunotherapy agent of the invention may be used in the method or uses of the invention.

The invention provides for a splice modulating oligonucleotide, or use thereof, as described or claimed herein, in an exosome formulation. Exosome formulations are useful in enhancing delivery to target tissues or target cell(s), for example, cancer cells.

The invention provides for a conjugate comprising the splice modulating oligonucleotide, or use thereof, as described or claimed herein, such as a conjugate comprising the splice modulating oligonucleotide covalently linked to a trivalent GaINAc moiety. GaINAc conjugation enhances delivery to target cells in the liver, such as hepatocytes.

### RNA Editing Embodiments of the Invention

Recently, antisense oligonucleotides have been developed that recruit endogenous human ADARs (adenosine deaminase acting on RNA) to edit endogenous transcripts in a simple and programmable way (Merkle et al., Nat Biotechnol. 2019 February; 37(2):133-138). Oligonucleotide designs which can be used to mediate RNA editing include

It will be understood that an alternative method of introducing a change to the peptide encoding sequence of a target RNA (such as mRNA), is RNA editing, which may be used to introduce a deletion, insertion or substitution (RNA editing event). A deletion or substitution may for example introduce a frame shift, resulting in a novel antigen protein sequence down-stream of the RNA editing event. Stop codons in the target RNA may be targeted (by deletion, insertion or substitution), the removal of the stop codon can result in novel antigenic sequence produce down-steam of the RNA editing event. Frame shifting RNA editing events can also result in the stop codon becoming out of frame, thereby also resulting in the production of novel antigen protein sequences.

In some embodiments the RNA editing oligonucleotide is capable of recruiting adenosine deaminase enzyme to the target RNA, resulting in an adenosine deaminase event. Adenosine deaminase results in the creation of an inosine base, which is read as a A→G substitution. In some advantageous embodiments, the RNA editing event is an A to I substitution. Such A→G substitutions can be used to:
i) Create a AUG start codon up upstream of the endogenous translation start point—this creates a peptide epitope at the N terminus of the protein product. For example an AUA triplet upstream of the endogenous AUG start codon may be RNA edited to become an alternative AUG start codon.
ii) Change the start AUG to IUG(GUG), causing the use of a new/novel AUG start codon downstream generating an out of frame translation causing a completely new peptide sequence.
iii) Alter the endogenous stop codon, resulting in translation past the endogenous stop codon. The stop codons UAG, UGA or UAA may therefore be edited in the RNA target to become UGG, encoding tryptophan. The translation past the endogenous stop codon results in a peptide epitope at the C terminus of the protein product.

The invention provides for a method for engineering a peptide epitope in a cell, said method comprising administration of an effective amount of a RNA editing oligonucleotide to the cell, wherein the RNA editing oligonucleotide targets a target RNA to insert, delete or substitute a nucleobase of the target RNA to produce an aberrant RNA transcript encoding an aberrant polypeptide containing the peptide epitope.

The invention provides for a method for engineering a peptide epitope in a cell, said method comprising administration of an effective amount of a RNA editing oligonucleotide to the cell, wherein the RNA editing oligonucleotide targets a target RNA substitute a nucleobase of the target RNA to produce an aberrant RNA transcript encoding an aberrant polypeptide containing the peptide epitope.

The invention provides for a method for engineering a peptide epitope in a cell, said method comprising administration of an effective amount of a RNA editing oligonucleotide to the cell, wherein the RNA editing oligonucleotide targets a target RNA to insert, delete or substitute a nucleobase of the target RNA to produce an aberrant RNA transcript encoding an aberrant polypeptide containing the peptide epitope.

The invention provides for a method for engineering a peptide epitope in a cell, said method comprising administration of an effective amount of a RNA editing oligonucleotide to the cell, wherein the RNA editing oligonucleotide targets a target RNA to insert, delete or substitute a nucleobase of the target RNA to produce an aberrant RNA transcript encoding an aberrant polypeptide containing the peptide epitope.

The invention provides for a method for engineering a peptide epitope in a cell, said method comprising administration of an effective amount of a RNA editing oligonucleotide to the cell, wherein the RNA editing oligonucleotide targets a target RNA to insert, delete or substitute a nucleobase of the target RNA to produce an aberrant RNA transcript encoding an aberrant polypeptide containing the peptide epitope.

The invention provides a method for engineering a peptide epitope, which may be referred to herein as a neoantigen peptide, in or a cell, said method comprising administration of an effective amount of a RNA editing oligonucleotide to the cell, wherein the RNA editing oligonucleotide targets a target RNA to insert, delete or substitute a nucleobase of the target RNA to produce an aberrant RNA transcript encoding an aberrant polypeptide containing the peptide epitope.

An adenosine to inosine substitution is a particularly advantageous substitution in the context of the RNA editing methods of the present invention as the inosine is recognized as a G nucleobase in translation*, and as such the A to I (also referred to as A to G*) enables the editing of start and stop codons, allowing for the introduction of new start or stop codons, or the deletion of existing start or stop codons.

The invention provides for a method of immune modulating a target cell in a subject, said method comprising the steps of:

a. Vaccinate the subject with an agent comprising a peptide epitope, or encoding peptide epitope;
b. Administer a RNA editing oligonucleotide to the subject, wherein the RNA editing oligonucleotide targets a target RNA in a target cell in the subject, and modulates the splicing of the target RNA to insert, delete or substitute a nucleobase of the target RNA to produce an aberrant RNA transcript encoding an aberrant polypeptide containing a peptide epitope;
to trigger or enhance the immune response by the subject to the peptide epitope, such as a target cell expressing the peptide epitope;
wherein step a. and step b: may be in the order of step a. and then step b., or step b. and then step a., or step a. and step b. are performed simultaneously.

The invention provides for a method of immune modulating a target cell in a subject, said method comprising the steps of:

a. administer a RNA editing oligonucleotide to the subject, wherein the RNA editing oligonucleotide targets a target RNA in a target cell in the subject, to insert, delete or substitute a nucleobase of the target RNA to produce an aberrant RNA transcript encoding an aberrant polypeptide containing a peptide epitope;
b. administer an antibody to the subject, wherein the antibody is specific for the peptide epitope
to trigger or enhance the immune response by the subject to the peptide epitope, such as a target cell expressing the peptide epitope; wherein step a. and step b: may be in the order of step a. and then step b., or step b. and then step a., or step a. and step b. are performed simultaneously.

The invention provides for a method of immune modulating a target cell in a subject, said method comprising the step of administering a RNA editing oligonucleotide to the subject, wherein the RNA editing oligonucleotide targets a target RNA in a target cell in the to insert, delete or substitute a nucleobase of the target RNA to produce an aberrant RNA transcript encoding an aberrant polypeptide containing a peptide epitope;

to trigger or enhance the immune response by the subject to the peptide epitope, such as a target cell expressing the peptide epitope.

The invention provides for a method of immunotherapy treatment of a disease in a subject, said method comprising the steps of

a. administer a RNA editing oligonucleotide to the subject, wherein the RNA editing oligonucleotide targets a target RNA in the target cell in the subject, to insert, delete or substitute a nucleobase of the target RNA to produce an aberrant RNA transcript encoding an aberrant polypeptide containing a peptide epitope;
b. administer an immunotherapy antibody to the subject, wherein the immunotherapy antibody is specific for the peptide epitope;
to trigger or enhance the immune response by the subject to the peptide epitope, such as the peptide epitope expressed by the target cell; wherein step a. and step b: may be in the order of step a. and then step b., or step b. and then step a., or step a. and step b. are performed simultaneously.

The invention provides method of immune modulating a target cell in a subject, said method comprising the administration of a RNA editing oligonucleotide to the subject, wherein the RNA editing oligonucleotide targets a RNA to insert, delete or substitute a nucleobase of the target RNA to produce an aberrant RNA transcript encoding an aberrant polypeptide containing a peptide epitope; wherein the aberrant epitope is immunogenic to the subject; to trigger or enhance the immune response by the subject to the target cell.

The invention provides a method of immune modulating a target cell in a subject, said method comprising the steps of:

a. Vaccinate the subject with an agent comprising a peptide epitope, or encoding a peptide epitope;
b. Administer a RNA editing oligonucleotide to the subject, wherein the RNA editing oligonucleotide targets a RNA to insert, delete or substitute a nucleobase of the target RNA to produce an aberrant RNA transcript encoding an aberrant polypeptide containing a peptide epitope;
to trigger or enhance the immune response by the subject to the target cell;
wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response.

In some embodiments, between steps a and b or b and a, an optionally waiting step c. may be employed, to e.g. allow the subject to develop an adaptive immune response to the antigen peptide (order of steps a, c, b), or to allow the expression of the epitope peptide on the target cell (order of steps b, c, a).

The invention provides for a method of immune modulating a target cell in a subject, said method comprising the steps of:
a. administer a RNA editing oligonucleotide to the subject, wherein the RNA editing oligonucleotide targets a RNA splice site in the target cell in the subject, to insert, delete or substitute a nucleobase of the target RNA to produce an aberrant RNA transcript encoding an aberrant polypeptide containing a peptide epitope;
b. administer an antibody to the subject, wherein the antibody is specific for peptide epitope
   to trigger or enhance the immune response by the subject to the target cell, wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response particularly when the antibody is administered in step b.

A waiting step c. may be performed between steps a and b, for example to allow for the expression of the peptide epitope in the target cell (order of steps a, b, c).

The invention provides for the use of a RNA editing oligonucleotide for the production of a peptide epitope in a cell.

The invention provides for the use of a RNA editing oligonucleotide in the immunotherapy treatment, e.g. of cancer, wherein the RNA editing oligonucleotide targets a RNA to insert, delete or substitute a nucleobase of the target RNA to produce an aberrant RNA transcript encoding an aberrant polypeptide containing a peptide epitope, in the cell e.g. in the cancer cell; wherein the immunotherapy treatment comprises the administration of an therapeutic antibody which recognizes the peptide epitope to the subject.

The invention provides for the use of a RNA editing oligonucleotide in the cancer vaccine therapy, wherein the RNA editing oligonucleotide targets a RNA to produce an aberrant RNA transcript encoding an aberrant polypeptide containing the peptide epitope in a cancer cell wherein the vaccine therapy results in the generation of, or enhances the immune response, by the subject to the peptide epitope.

### BRIEF DESCRIPTION OF FIGURES

FIG. 1. Schematic representation of splice modulating events induced in pre-mRNA by oligonucleotides. Exon junctions are indicated by dashed lines, while novel peptide sequences are represented by red circles. For simplicity, single splice modulating events only are depicted.

FIG. 2. A novel splice junction between exon 6 and exon 8 in CEMIP mRNA is induced by specific oligonucleotides. CEMIP exon 7 skipping events are measured as percentage of the total level of CEMIP transcript.

FIG. 3. A novel splice junction between exon 27 and exon 29 in CEMIP mRNA is induced by specific oligonucleotides. CEMIP exon 28 skipping events are measured as percentage of the total level of CEMIP transcript.

FIG. 4. A novel splice junction between exon 7 and exon 9 in ETV4 mRNA is induced by specific oligonucleotides. ETV4 exon 8 skipping events are measured as percentage of the total level of ETV4 transcript.

FIG. 5. A novel splice junction between exon 9 and exon 11 in ETV4 mRNA is induced by specific oligonucleotides. ETV4 exon 10 skipping events are measured as percentage of the total level of ETV4 transcript.

FIG. 6. Scatter-plot depicting relative PARPBP gene expression level in 56 lung squamous cell carcinomas clinical samples (triangles) and a collection of normal human tissues (circles).

FIG. 7. Box-plot depicting gene expression level (TPM) of PARPBP among human healthy tissues from GTEX database.

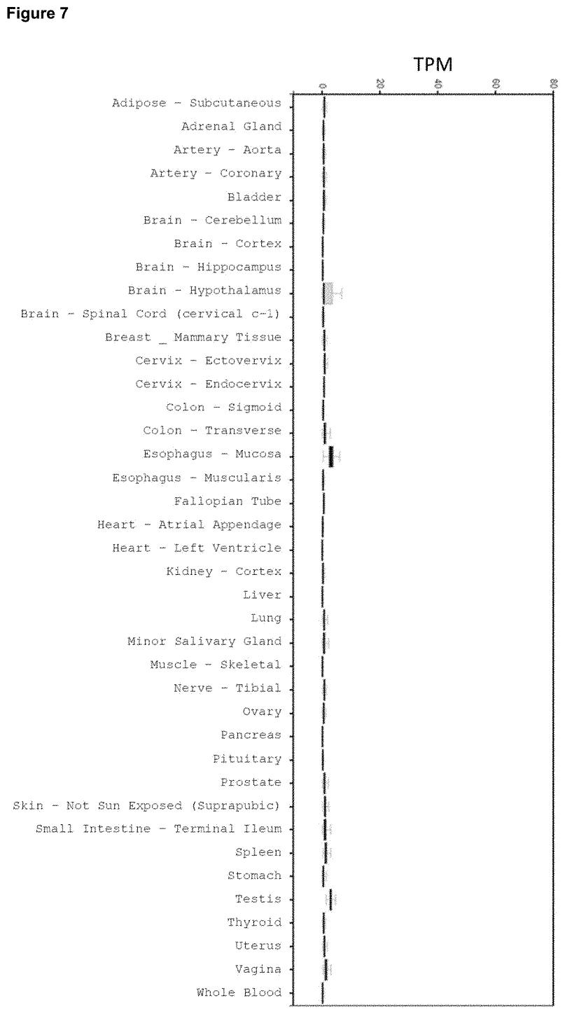

FIG. 8. Visualization of a capillary electrophoresis immunoassay detection of CEMIP and GAPDH in colo-205 cells treated with oligonucleotide inducing CEMIP exon 28 skipping. First 4 lanes contains lysate before CEMIP protein was enriched using immune precipitation shown in the last 4 lanes. The measured molecular weight estimated by the WES machine is indicated.

FIG. 9. Visualization of a capillary electrophoresis immunoassay detection of novel CEMIP c-terminus induced by oligonucleotide inducing CEMIP exon 28 skipping. The concentration of O195 is indicated above the lane. The measured molecular weight estimated by the WES machine is indicated. HTPR1 antibody was used as loading control.

FIG. 10. The top panel shows the identified MS/MS spectrum corresponding to 11 amino acids contained within the 15 amino acid Wild type CEMIP C-terminus IFQVVPIPVVKKKKL (SEQ ID NO 299) identified in samples both with and without O195 treatment. The lower panel shows the identified MS/MS spectrum of the last 11 aminoacids of the predicted novel c-terminus K A N G I R W L Q R Q L P A H L G D T G H, SEQ ID NO 189. This peptide fragment was only identified in samples treated with O195.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide

### RNA Editing Oligonucleotides

An RNA editing oligonucleotide is an oligonucleotide which is capable of targeting the target RNA via hybridization between a contiguous nucleotide sequence of an RNA editing oligonucleotide, and thereby result in an insertion, deletion or substitution of one or more nucleobases within the target RNA, typically over the region of complementarity between the contiguous nucleotide sequence of the RNA editing oligonucleotide and the RNA target sequence. The RNA editing oligonucleotide may comprise a further region (other than the contiguous nucleotide sequence), which allows for the recruitment of an RRE editing enzyme. The further region may for example comprise a double stranded region. One advantageous form of RNA editing oligonucleotide are ADAR recruiting oligonucleotides, for example as disclosed in Merkle et al., Nat Biotechnol. 2019 February; 37(2):133-138—see also WO17010556. RNA editing methods and RNA editing oligonucleotides agents are disclosed in WO19084063, WO19071274. WO18161032, WO18134301, WO18041973, WO17220751, WO16097212. RNA editing may also be achieved by CRISPR/Cas9 editing RNA editing—see WO18208998 for example.

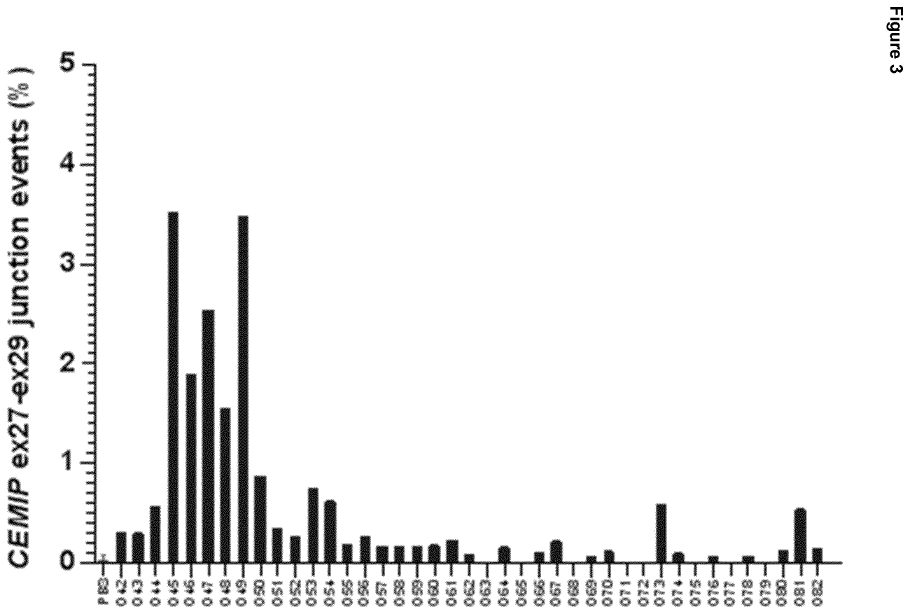

By way of example, as disclosed in Merkle et al., the ADAR recruiting oligonucleotides may comprise a 3′ region of modified nucleotides, e.g. 10-25 nucleotide in length, which comprise a C nucleoside at the position of the A base on the target RNA (which is to be edited to an I, read as a G, nucleobase), but is otherwise complementary to the target RNA (fully complementary except for the mismatch at the C nucleoside). The C nucleoside is positioned within the 3′region and typically is not a 3′ terminal nucleoside. The C nucleoside and the nucleosides flanking the C nucleoside may be RNA nucleotides, and the remainder of the 3′ region may be for example 2′-O-methyl nucleosides or other 2′-O-alkyl nucleosides. By way of example the C nucleoside may have an adjacent RNA nucleoside and a further 6-12 2′-O-methyl nucleosides positioned 3′ to the C nucleoside. There C nucleoside may be flanked by a single RNA nucleoside, and a further 4-8 2′-O-methyl nucleosides. The 3′ terminus may be protected from nucleases, for example by the use of phosphorothioate internucleoside linkages between the terminal 2-6 nucleosides, e.g. a region of 2-6 2′-O-methyl phosphorothioate linked nucleosides. The 3′region may be 10-25 nucleosides in length, such as 15-20, such as 16, 17, 18 or 19 nucleosides in length. The RNA editing oligonucleotide may further comprise a 5′ ADAR recruiting region—the ADAR recruiting region is typically independent of the target sequence (i.e. does not rely on complementarity to the target RNA), and typically comprises a double stranded region of modified nucleosides, wherein the double stranded region may comprise one or two mismatched nucleotide pairs (non-base pairing nucleosides). The double stranded region may be formed by a hairpin structure, i.e. the oligonucleotide is a single oligonucleotide where the 5′ region forms a hairpin, forming the double stranded region. Alternatively, the 5′ ADAR recruiting region may be formed by two complementary oligonucleotide molecules. The double stranded region may for example be 15-30 base pairs in length, such as 22-27 or 25 base pairs, including for example 1 or 2 non pairing bases. Suitably the double stranded region comprises modified nucleosides, such as 2′-O-methyl, LNA and/or 2′-O-MOE nucleosides. By way of example, see FIG. 3a of Merkle et al., Nat Biotechnol. 2019 February; 37(2):133-138.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. Adventurously, the contiguous nucleotide sequence is 100% complementary to the target nucleic acid.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target nucleic acid or target sequence). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (e.g. in the contiguous nucleotide sequence of the compound of the invention and in the reference sequence), dividing that number by the total number of nucleotides in the aligned region and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RTIn(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Sequence

The term “target sequence” as used herein refers to a sequence of nucleotides present in the target nucleic acid (RNA) which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence which are complementary to a target sequence present in the target nucleic acid molecule. The contiguous nucleotide sequence (and therefore the target sequence) comprises of at least 10 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

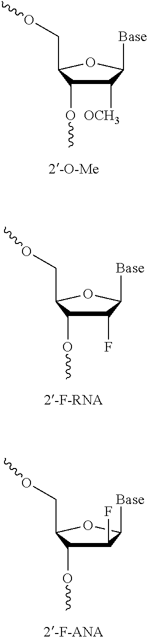

In relation to the present invention 2′ substituted does not include 2′ bridged molecules like LNA.

### Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of

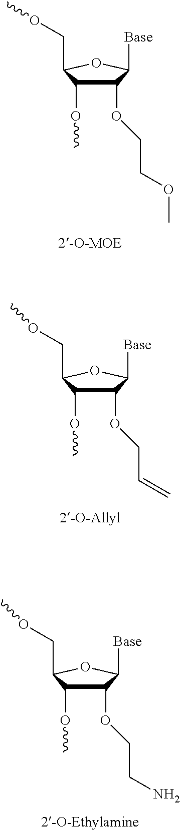

the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

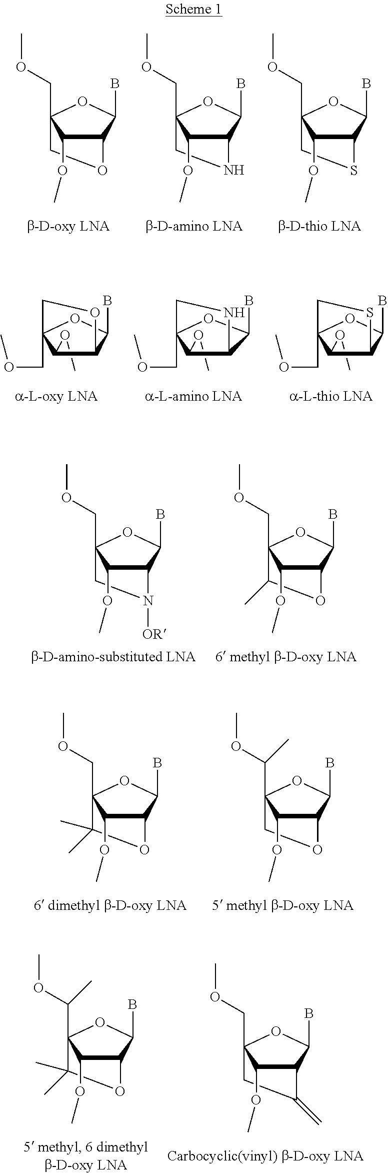

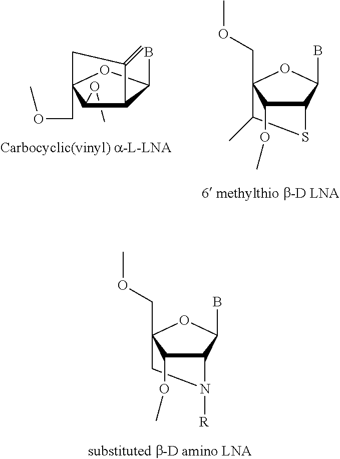

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

### Morpholino Oligonucleotides

In some embodiments, the oligonucleotide of the invention comprises or consists of morpholino nucleosides (i.e. is a Morpholino oligomer and as a phosphorodiamidate Morpholino oligomer (PMO)). Splice modulating morpholino oligonucleotides have been approved for clinical use—see for example eteplirsen, a 30nt morpholino oligonucleotide targeting a frame shift mutation in DMD, used to treat Duchenne muscular dystrophy. Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

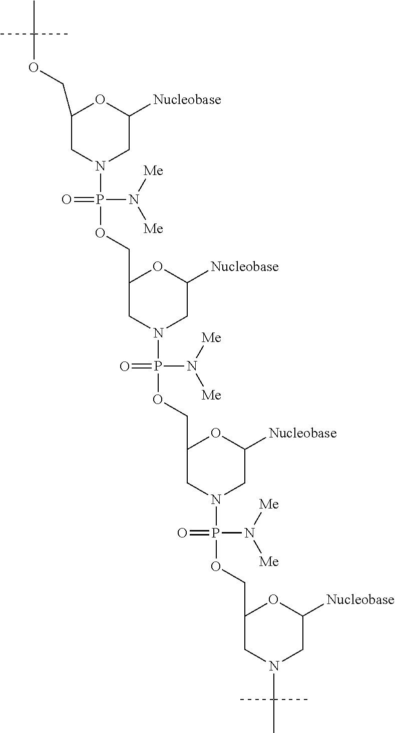

In some embodiments, morpholino oligonucleotides of the invention may be, for example 20-40 morpholino nucleotides in length, such as morpholino 25-35 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

### Splice Modulating Oligonucleotides

Splice modulating refers to the ability of an agent, such as an antisense oligonucleotide to alter the splicing events in a target RNA, such as a pre-MRNA. Splice modulating oligonucleotides may hybridise to and be complementary to intron/exon boundaries, or to cis-elements which regulate or control splice events, these are collectively referred to herein as splice sites, or splice regulatory elements, regions or sequences. Splice switching oligonucleotides is a term commonly used in the art to refers to splice modulating oligonucleotides.

Numerous designs for splice modulating oligonucleotides are known in the art, for example, see WO2007/028065, which discloses chimeric oligomeric compounds 13 to 80 nucleotides in length and WO2007/058894 which refers to LNA mixmer antisense oligonucleotides for splice modulating the TNFR2 transcript, resulting in a soluble form of TNFR2. Further splice modulator antisense oligonucleotides designs are disclosed in, for example: Sazani et al., Antisense and Nucleic Acid Drug Dev. 13:119-128 (2003): Childs et al., PNAS Aug. 20, 2002: 99 11091-11096 discloses a LNA/DNA mixmers for Oligonucleotide directed inhibition of Candida albicans group I intron splicing; Sazani et al., Nature Biotechnology 2002 1228-1233 discloses fully 2′-O-MOE modified phosphorothioate splice modulators which correct the splicing of an aberrant GFP reporter gene in mice: Roberts et al., MOLECULAR THERAPY Vol. 14, No. 4, October 2006 pp 471-475 reports on LNA mixmers for splice modulation of the aberrant GFP reporter gene in mice. Aartsma-Rus et al., Gene Therapy (2004) 11, 1391-1398 refers to a comparative analysis of antisense oligonucleotide analogs for targeted DMD exon 46 skipping in muscle cells, using LNA, 2-O-Methyl and morphilino exon skipping antisense oligonucelotides. Hua et al., PLoS Biology April 2007 | Volume 5 | Issue 4 | e73 refers to the enhancement of SMN2 Exon 7 Inclusion by antisense oligonucleotides targeting the Exon. Havens and Hastings Nucleic Acids Research, 2016 1 doi: 10.1093/nar/gkw533 reviews antisense modulation of splicing; WO2007/047913 refers to method for identifying cis-splicing elements which may be target sites for modulating splicing events.

Antisense oligonucleotides for modulation of splicing preferably operate through a non-RNAseH mediated mechanism (they may therefore advantageously be referred to as RNaseH independent). In some embodiments, the antisense oligonucleotide splice modulator is not capable of recruiting RNaseH. In some embodiments the antisense oligonucleotide splice modulator does not comprise more than 3 contiguous DNA nucleotides or does not comprise more than 4 contiguous DNA nucleotides.

Oligonucelotides used for splice modulation may for example comprise a contiguous sequence of nucleotides of 8-40 nucleotides which are complementary to the target RNA. Oligonucelotides used for splice modulation may for example comprise a contiguous sequence of nucleotides of 8-30 nucleotides which are complementary to the target RNA.

Splice modulating oligonucleotides, or the contiguous nucleotide sequence thereof may be, for example between 8-30 nucleotides in length, such as 12-24 nucleotides, such as 13, 14, 15, 16, 17, 18, 19, 20, 21, 22 or 23 nucleotides in length.

### LNA Splice Modulators

Due to their remarkable high affinity for RNA targets, LNA oligonucleotides make highly effective splice modulators.

In some embodiments the antisense oligonucleotide comprises at least one LNA nucleoside—these may be referred to as LNA splice modulators. In some embodiments, the antisense oligonucleotide comprises both DNA and LNA nucleosides, optionally also comprising one or more 2-O-MOE nucleosides (referred to herein as LNA mixmers). In some embodiments, the antisense oligonucleotide comprises both one 2′-O-methyoxyethyl nucleosides and LNA nucleosides, optionally also comprising one or more DNA nucleosides. In some embodiments the LNA antisense oligonucleotide does not comprise DNA or RNA nucleotides (a totalmer). In some embodiments all the nucleosides with the antisense oligonucleotide or contiguous nucleotide sequence thereof are independently LNA, 2′-O-methoxyethyl or DNA nucleosides. In some embodiments all the nucleosides with the antisense oligonucleotide or contiguous nucleotide sequence thereof are independently LNA or DNA nucleosides. In some the antisense oligonucleotide or contiguous nucleotide sequence thereof comprises both LNA and DNA nucleosides, or all of the nucleotides with the contiguous nucleotide sequence are LNA or DNA nucleotides.

The LNA splice modulators described herein may further comprise one or more phosphorothioate internucleoside linkage. In some embodiments, all of the internucleoside linkages in the antisense oligonucleotide or contiguous nucleotide sequence thereof are phosphorothioate internucleoside linkages. In some embodiments, the 2′-O-MOE splice modulators are between 10-30 nucleotides in length, such as 12-20 nucleotides in length.

### 2′-O-MOE Splice Modulators

In some embodiments the antisense oligonucleotide comprises at least one 2′-O-methyoxyethyl nucleoside—these may be referred to as 2′-O-MOE splice modulators. In some embodiments, the antisense oligonucleotide comprises both one 2′-O-methyoxyethyl nucleosides and LNA nucleosides, optionally also comprising one or more DNA nucleosides. In some embodiments all the nucleosides with the antisense oligonucleotide or contiguous nucleotide sequence thereof are 2′-O-methoxyethyl nucleosides. In some embodiments all the nucleosides with the antisense oligonucleotide or contiguous nucleotide sequence thereof are 2′-O-methoxyethyl nucleosides. The 2′-O-MOE splice modulators described herein may further comprise one or more phosphorothioate internucleoside linkage. In some embodiments, all of the internucleoside linkages in the antisense oligonucleotide or contiguous nucleotide sequence thereof are phosphorothioate internucleoside linkages. IN some embodiments, the 2′-O-MOE splice modulators are between 12-30 nucleotides in length. For fully 2-O-MOE oligonucleotides lengths of a least 18 nucleotides are preferred to provide sufficient affinity for the target RNA.

### Totalmers

In some embodiments, the oligomer or contiguous nucleotide sequence thereof consists of a contiguous sequence of nucleotide nucleoside analogues, such as affinity enhancing nucleotide nucleoside analogues—referred to herein is as a ‘totalmer’.

A totalmer is a single stranded oligomer, or contiguous nucleotide sequence thereof, which does not comprise DNA or RNA nucleosides, and as such only comprises nucleoside analogue nucleosides. only comprises non-naturally occurring nucleotides.

The oligomer, or contiguous nucleotide sequence thereof, maybe a totalmer—indeed various totalmer designs are highly effective as therapeutic oligomers, particularly when used as splice switching oligomers (SSOs).

In some embodiments, the totalmer comprises or consists of at least one XYX or YXY sequence motif, such as a repeated sequence XYX or YXY, wherein X is LNA and Y is an alternative (i.e. non LNA) nucleotide analogue, such as a 2′-OMe RNA unit and 2′-fluoro DNA unit. The above sequence motif may, in some embodiments, be XXY, XYX, YXY or YYX for example.

In some embodiments, the totalmer may comprise or consist of a contiguous nucleotide sequence of between 8 and 16 nucleotides, such as 9, 10, 11, 12, 13, 14, or 15 nucleotides, such as between 8 and 12 nucleotides.

In some embodiments, the contiguous nucleotide sequence of the totaolmer comprises of at least 30%, such as at least 40%, such as at least 50%, such as at least 60%, such as at least 70%, such as at least 80%, such as at least 90%, such as 95%, such as 100% LNA units. The remaining units may be selected from the non-LNA nucleotide analogues referred to herein in, such as those selected from the group consisting of 2′-O_alkyl-RNA unit, 2′-OMe-RNA unit, 2′-amino-DNA unit, 2′-fluoro-DNA unit, LNA unit, PNA unit, HNA unit, INA unit, and a 2′MOE RNA unit, or the group of 2′-OMe RNA unit and 2′-fluoro DNA unit. In some embodiments the totalmer consist or comprises of a contiguous nucleotide sequence which consists only of LNA units.

### Mixmers

The term ‘mixmer’ refers to oligomers, or contiguous nucleotide sequences thereof, which comprise DNA nucleosides and nucleoside analogue nucleosides, both naturally and non-naturally occurring nucleotides, where, as opposed to gapmers, tailmers, headmers and blockmers, there is no contiguous sequence of more than 5 naturally occurring DNA nucleotidesnucleosides, such as DNA units.

The oligomer, or contiguous nucleotide sequence thereof, according to the invention may be mixmers—indeed various mixmer designs are highly effective as therapeutic oligomers, particularly when splice modulating/splice switching oligomers (SSOs).

The oligomer may, or contiguous nucleotide sequence thereof, in some embodiments, also be a mixmer and indeed, due to the ability of mixmers to effectively and specifically bind to their target, the use of mixmers as therapeutic oligomers are considered to be particularly effective in decreasing the target RNA.

In some embodiments, the mixmer comprises or consists of a contiguous nucleotide sequence of repeating pattern of nucleotide analogue and naturally occurring nucleotides, or one type of nucleotide analogue and a second type of nucleotide analogues. The repeating pattern, may, for instance be every second or every third nucleotide is a nucleotide analogue, such as LNA, and the remaining nucleotides are naturally occurring nucleotides, such as DNA, or are a 2′substituted nucleotide analogue such as 2′MOE of 2′fluoro analogues as referred to herein, or, in some embodiments selected form the groups of nucleotide analogues referred to herein. It is recognised that the repeating pattern of nucleotide analogues, such as LNA units, may be combined with nucleotide analogues at fixed positions—e.g. at the 5′ or 3′ termini.

In some embodiments the first nucleotide of the oligomer or mixmer, counting from the 3′ end, is a nucleotide analogue, such as an LNA nucleotide.

In some embodiments, which maybe the same or different, the second nucleotide of the oligomer or mixmer, counting from the 3′ end, is a nucleotide analogue, such as an LNA nucleotide.

In some embodiments, which maybe the same or different, the seventh and/or eighth nucleotide of the oligomer or mixmer, counting from the 3′ end, are nucleotide analogues, such as LNA nucleotides.

In some embodiments, which maybe the same or different, the ninth and/or the tenth nucleotides of the oligomer or mixmer, counting from the 3′ end, are nucleotide analogues, such as LNA nucleotides.

In some embodiments, which maybe the same or different, the 5′ terminal of the foligomer or mixmer is a nucleotide analogue, such as an LNA nucleotide.

The above design features may, in some embodiments be incorporated into the mixmer design, such as mixmers splice modulating oligonucleotides.

In some embodiments, the mixmer does not comprise a region of more than 4 consecutive DNA nucleotide units or 3 consecutive DNA nucleotide units. In some embodiments, the mixmer does not comprise a region of more than 2 consecutive DNA nucleotide units.

In some embodiments, the mixmer comprises at least a region consisting of at least two consecutive nucleotide analogue units, such as at least two consecutive LNA units. In some embodiments, the mixmer comprises at least a region consisting of at least three consecutive nucleotide analogue units, such as at least three consecutive LNA units. In some embodiments, the mixmer of the invention does not comprise a region of more than 7 consecutive nucleotide analogue units, such as LNA units. In some embodiments, the mixmer of the invention does not comprise a region of more than 6 consecutive nucleotide analogue units, such as LNA units. In some embodiments, the mixmer of the invention does not comprise a region of more than 5 consecutive nucleotide analogue units, such as LNA units. In some embodiments, the mixmer of the invention does not comprise a region of more than 4 consecutive nucleotide analogue units, such as LNA units. In some embodiments, the mixmer of the invention does not comprise a region of more than 3 consecutive nucleotide analogue units, such as LNA units. In some embodiments, the mixmer of the invention does not comprise a region of more than 2 consecutive nucleotide analogue units, such as LNA units.

### Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

### GaINAc Conjugates

Conjugate moieties capable of binding to the asialoglycoprotein receptor (ASGPRr) are particular useful for targeting hepatocytes in liver, and are therefore advantageous. In some embodiments the invention provides a conjugate comprising the oligonucleotide of the invention and an asialoglycoprotein receptor targeting conjugate moiety. The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. JB. C. 1996, 271, 6686) or are readily determined using methods typical in the art.

In one embodiment the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GaINAc).

To generate the ASGPR conjugate moiety the ASPGR targeting moieties (preferably GaINAc) can be attached to a conjugate scaffold. Generally the ASPGR targeting moieties can be at the same end of the scaffold. In one embodiment the conjugate moiety consists of two to four terminal GaINAc moieties linked to a spacer which links each GaINAc moiety to a brancher molecule that can be conjugated to the antisense oligonucleotide.

In a further embodiment the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GaINAc) moieties.

The ASPGR targeting scaffold which constitute the conjugate moiety can for example be generated by linking the GaINAc moiety to the spacer through its C-I carbon. A preferred spacer is a flexible hydrophilic spacer (U.S. Pat. No. 5,885,968; Biessen et al. J. Med. Chern. 1995 Vol. 39 p. 1538-1546). A preferred flexible hydrophilic spacer is a PEG spacer. A preferred PEG spacer is a PEG3 spacer. The branch point can be any small molecule which permits attachment of two to three GaINAc moieties or other asialoglycoprotein receptor targeting moieties and further permits attachment of the branch point to the oligonucleotide, such constructs are termed GaINAc clusters or GaINAc conjugate moieties. An exemplary branch point group is a di-lysine. A di-lysine molecule contains three amine groups through which three GaINAc moieties or other asialoglycoprotein receptor targeting moieties may be attached and a carboxyl reactive group through which the di-lysine may be attached to the oligomer. Khorev, et al 2008 Bioorg. Med. Chem. Vol 16, pp. 5216 also describes the synthesis of a suitable trivalent brancher. Other commercially available branchers are 1,3-bis-[5-(4,4′-dimethoxytrityloxy)pentylamido]propyl-2-[(2-cyanoethyl)-(N,N-diisopropyl)] phosphoramidite (Glen Research Catalogue Number: 10-1920-xx); tris-2,2,2-[3-(4,4′-dimethoxytrityloxy)propyloxymethyl]ethyl-[(2-cyanoethyl)-(N,N-diisopropyl)]-phosphoramidite (Glen Research Catalogue Number: 10-1922-xx) and tris-2,2,2-[3-(4,4′-dimethoxytrityloxy)propyloxymethyl]methyleneoxypropyl-[(2-cyanoethyl)-(N,N-diisopropyl)]-phosphoramidite; and 1-[5-(4,4′-dimethoxy-trityloxy)pentylamido]-3-[5-fluorenomethoxy-carbonyl-oxy-pentylamido]-propyl-2-[(2-cyanoethyl)-(N,N-diisopropyl)]-phosphoramidite (Glen Research Catalogue Number: 10-1925-xx).

Other GaINAc conjugate moieties can include, for example, those described in WO 2014/179620 and WO 2016/055601 and PCT/EP2017/059080 (hereby incorporated by reference), as well as small peptides with GaINAc moieties attached such as Tyr-Glu-Glu- (aminohexyl GaINAc)3 (YEE(ahGaINAc)3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GaINAc conjugate moiety, may be attached to the 3′- or 5′-end of the oligonucleotide using methods known in the art. In one embodiment the ASGPR conjugate moiety is linked to the 5′-end of the oligonucleotide. In some embodiments the conjugate moiety is a tri-valent N-acetylgalactosamine (GaINAc), such as those shown below:

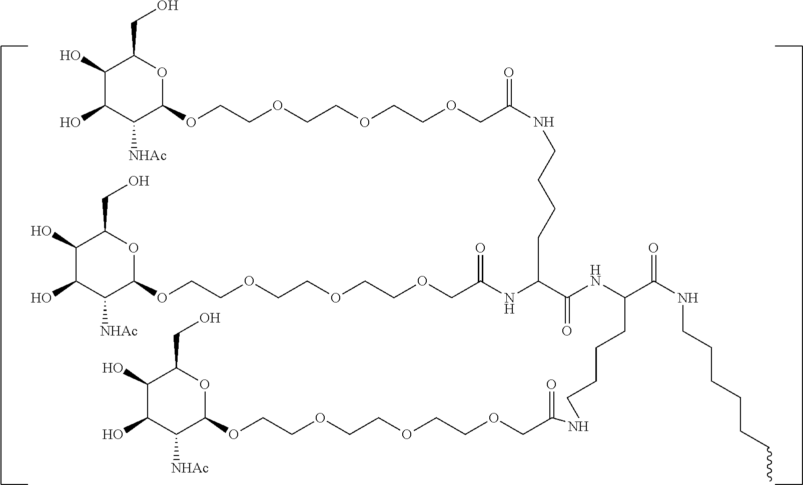

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. DNA phosphodiester containing biocleavable linkers are described in more detail in WO 2014/076195 (hereby incorporated by reference)—see also region D′ or D″ herein.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### Administration

The oligonucleotides or pharmaceutical compositions of the present invention may be administered topical or enteral or parenteral (such as, intravenous, subcutaneous, intramuscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

### DETAILED DESCRIPTION

The invention provides a method for engineering a peptide epitope, which may be referred to herein as a neo-antigen peptide, in or a cell, said method comprising administration of an effective amount of a splice modulating oligonucleotide to the cell, wherein the splice modulating oligonucleotide targets an RNA splice event to modulate the splicing of the RNA (referred to herein as the target RNA) at the splice site to produce an aberrant RNA transcript encoding an aberrant polypeptide containing the peptide epitope.

The method may be an in vitro method or an in vivo method.

For in vivo use, the expression of the peptide epitope (neoantigen) may be used to induce or enhance an immune response.

### The Peptide Epitope

The peptide epitope may be displayed on the cell surface, for example via the major histocompatibility complex, e.g. MHC class I or II, or via a membrane anchoring domain within the aberrant polypeptide, or may in some embodiments be part of a secreted polypeptide.

In some embodiments the peptide epitope may be a novel peptide epitope which the cell does not synthesis without the modulation of the splicing event. However it is also envisaged that the peptide epitope may be expressed at a low level in the cell, (possibly a rare event or below the level of detection) and the methods or uses of the invention result in an effective enhancement of expression of the modulated splice event and production of the peptide epitope. In this context, the splicing event modulated in the present methods or uses is typically either not usually detectable or a rare event. In some embodiments, the aberrant RNA is either absent in the cell in the absence of the splice modulating oligonucleotide, or represents less than 0.1% of the RNA population originating for the target gene, such as less than 0.01% or less than 0.001%.

In some embodiments the peptide epitope is secreted from the cell.

In some embodiments the peptide epitope is presented on the cell as a MHC Class I or II molecule (or both).

In some embodiments the polypeptide containing the peptide epitope further comprises a membrane binding domain. The use of a target RNA which encodes a membrane binding domain (such as a transmembrane domain) may be that the peptide epitope is presented on the external surface of the cell.

It will be understood that the presentation of the peptide epitope on or secretion from the cell may occur via a combination of the above mechanisms or any other mechanism.

In some embodiments, the peptide epitope is SEQ ID NO 188: R T S R L C C C P G I Q I V P D R A W R I F Q C F F V Q.

In some embodiments, the peptide epitope is SEQ ID NO 189: K A N G I R W L Q R Q L P A H L G D T G H

In some embodiments, the peptide epitope is SEQ ID NO 190: Q E Q T D F A Y D S G Y G Y E K P L R P

In some embodiments, the peptide epitope is SEQ ID NO 191: G R Q A L G H P E E P A S H E L R Q A E P L A P I L L,

In some embodiments, the peptide epitope is SEQ ID NO 192: H C R G S N T V S D K D A T D

The invention provides for a vaccine which comprises the peptide epitope, such as a peptide epitope selected from the group SEQ ID NO 188, 189, 190, 191, and 192.

The invention provides for a polypeptide which is or comprises the peptide, such as a peptide selected from the groups SEQ ID NO 188, 189, 190, 191 and 192; and it's use in therapy, such as for use as a vaccine or in immunotherapy.

### The Target RNA

The target RNA may be any peptide or polypeptide encoding RNA, such as advantageously a pre-mRNA. It is well known that certain cancers are associated with the production of fusion transcripts (e.g certain Sarcomas, for example see Hofvander et al., Laboratory Investigation volume 95, pages 603-609 (2015)). In some embodiments the target RNA may be a fusion transcript.

Whilst IncRNAs are usually thought as non-coding, it is apparent that they often encode for short polypeptides (see for example Rion et al., Cell Research volume 27, pages 604-605 (2017)). In some embodiments, the target RNA is a IncRNA, which is either a peptide encoding IncRNA, or a IncRNA where the modulation of splicing results in the translation of a aberrant polypeptide.

In some embodiments the RNA target is a pre-mRNA which is over-expressed in the cancer cell (e.g. Cancer Genome Atlas TCGA).

Examples of target RNAs (and non limiting examples of cancer types where they are over-expressed): CEMIP (colon cancer), ETV4 (colon cancer), LRG5 (colon cancer), NOX1 (colon cancer), FOXP3 (T-REGS), IGF2BP3 (general cancer), MAGE-A4 (general cancer), NY-ESO-1 (general cancer), EWSR1 (sarcoma, aberrant splicing), FUS (sarcoma, aberrant splicing), SS18 (sarcoma, aberrant splicing).

### Modulation of Splicing

Splice modulation may be achieved by use of antisense oligonucleotides targeting intron/exon splice sites or the regions adjacent to the splice sites or cis-acting elements, or other splicing control regions (referred to collectively and interchangeably as splice regulatory elements, regions or sequences).

With the advent of global RNA sequencing technologies, potential splice modulating oligonucleotides may be screened in a suitable cell system to identify splice modulating oligonucleotides which are effective in modulating the splicing event as well as those which result in the production of an aberrant RNA encoding an aberrant polypeptide.

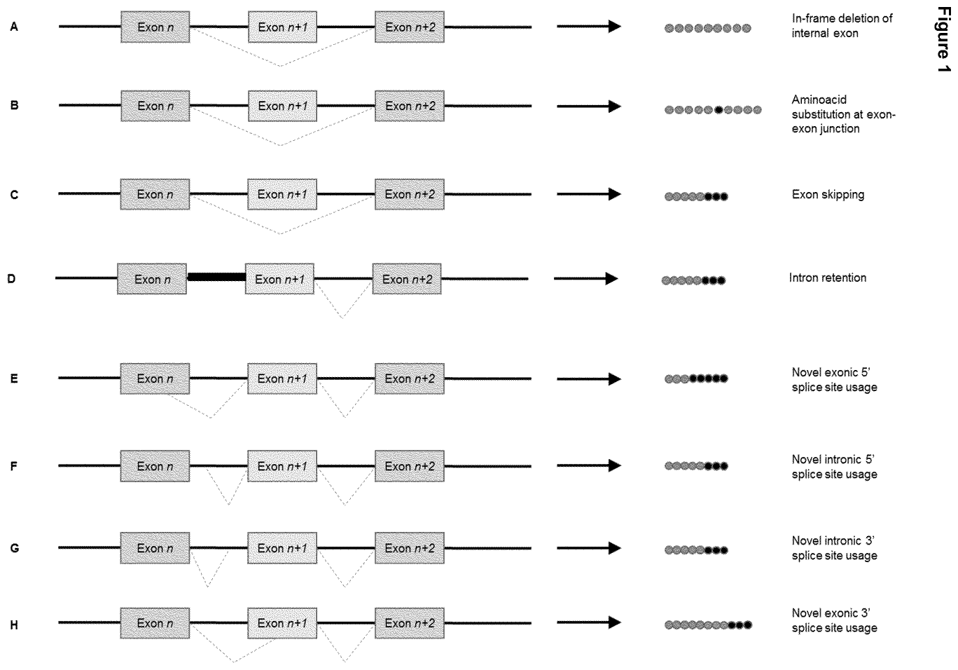

Modulation of splicing may be achieved by the use of an antisense oligonucleotide targeting a splice site of the RNA target, such oligonucleotides modulate alternative splicing by hybridizing to pre-mRNA sequences involved in splicing, and are also referred to as splice switching oligonucleotides. In order to modulate splicing of a target RNA, splice modulators may be designed to be complementary to or near intron/exon boundaries, or cis-elements which regulate the splicing event (See for example FIG. 1 which illustrates splice modulating events which can be effected by antisense oligonucleotides).

### Examples of Splice Modulation: (See FIG. 1 for Examples)

Numerous examples of splice modulating events are represented in FIG. 1, non-limiting examples of splice modulating events which result in the change of the peptide sequence of encoded by the target RNA include splice skipping, splice adding and splice shifting.

Splice skipping: Splice skipping refers to the modulation where the splicing is modulated by the skipping of at least one exon region (or part of an exon), and optionally intron regions, of the target RNA (e.g. pre-mRNA), resulting in a new or aberrant polypeptide sequence encoded from the region of the RNA (e.g. mRNA). Skipping may be enabled by activating a cryptic splice site or alternative 3′ or 5′ splice site.

In some embodiments, the splice modulating oligonucleotide modulates the of splicing of the target RNA, such as a pre-mRNA, to produce an aberrant RNA transcript introduced by the modulated splicing event, wherein the aberrant RNA (such as mRNA) transcript encodes an internal polypeptide deletion, to produce an aberrant polypeptide comprising an aberrant peptide sequence at the modulated splicing event (e.g. by skipping one or more exons), to produce the peptide epitope.

Splice Adding: Splice adding refers to refers to the modulation where the splicing is modulated by the inclusion of at least one codon originating from an intronic region, resulting in the addition of codons into the polypeptide chain, resulting in a new or aberrant polypeptide sequence encoded from the region of the RNA (e.g. mRNA). Splice adding may be enabled by activating a cryptic splice site or alternative 3′ or 5′ splice site. In some embodiments, the addition results a codon frame shift (see shifting) or may result in the retention of the same codon frame.

In some embodiments, the splice modulating oligonucleotide modulates the splicing of the target RNA, such as a pre-mRNA, to produce an aberrant RNA transcript (such as a mRNA) introduced by the modulated splicing event, wherein the aberrant RNA transcript encodes one or more codons from an intronic region of the target RNA, to produce an aberrant polypeptide comprising an aberrant peptide sequence which includes at least one or more peptide(s) encoded by the one or more codons originating from the intronic region, to produce the peptide epitope.

Splice Shifting: Splice shifting refers to the modulation where the splicing is modulated by the inclusion of or deletion of part of a codon, resulting in the introduction of a frame shift. At and down-stream of the frame shift this will result in the production of an aberrant polypeptide sequence, and optionally further down-stream a stop codon (may be a premature stop codon or a “delayed” stop codon).

In some embodiments, the splice modulating oligonucleotide modulates the of splicing of the target RNA, such as a pre-mRNA, to produce an aberrant RNA transcript comprising a codon frame shift introduced by the modulated splicing event, wherein the aberrant RNA transcript produces a polypeptide with a C-terminal region of at least 1 amino acid, which is transcribed from the region of the aberrant RNA transcript at or 3′ to the codon frame shift.

Peptide Epitopes Originating from Codon Frame Shifts (Splice Shifting) In some embodiments, the splice modulating oligonucleotide modulating splicing of the pre-mRNA (e.g. at the splice site or splice regulatory region) to produce an aberrant mRNA transcript with a codon frame shift introduced at the modulated splice site, wherein the aberrant mRNA transcript produces a polypeptide with a C-terminal region of at least 1 amino acid which is transcribed from the region of the aberrant mRNA transcript at or 3′ to the codon frame shift. In some embodiments the length of the C-terminal region is transcribed from the region of the aberrant mRNA transcript at or 3′ to the codon frame shift is at least 8 amino acids in length, such as at least 9 or at least 10 amino acids in length, such as 8, 9, 10, 11, 12, 13, or 14 amino acids. It will be understood that the peptide epitope may, in some embodiments, be formed by the combination of the N terminal region of the polypeptide encoded by the region of the aberrant RNA upstream of the codon frames shift in combination with the C-terminal region encoding at or down-stream of the codon frame shift. Alternatively the peptide epitope may be formed from the C-terminal region encoding at or down-stream of the codon frame shift.

The peptide epitope may be presented at the cell surface, e.g. via a major histocompatibility complex, or via the use of a target RNA which encodes an upstream membrane binding domain. Alternatively the peptide epitope may be secreted. The use of splice modulating oligonucleotides to enhance the secretion of isoforms of polypeptides is well known (e.g. TNFR2) and it is therefore envisaged that the methods of the invention may also result in peptide epitopes being both presented at the cell surface and secreted—indeed this may be highly advantageous when triggering or enhancing an immune response to the peptide epitope.

### The Cell

The cell referred to in the context of the present invention may be in vitro or in vivo, and may be a cell which is associated with a disease phenotype, for example a cancer cell, which is expressing the target RNA. In some embodiments, the cell is over-expressing the target RNA as compared to a cell originating from the same tissue which is not associated with the disease phenotype. The examples provide illustrative methods of how such target RNAs may be identified.

In some embodiments, the cell is a cancer cell, such as a tumor cell, for example a colon cancer cell, metastasized colon cancer cell or metastasized colon cancer cell in the liver.

In some embodiments the cancer, or cancer cell is selected from the group consisting of bladder cancer, breast cancer, colon cancer, colorectal cancer, endometrial cancer, kidney cancer, leukemia, liver cancer, lung cancer, melanoma, lymphoma, pancreatic cancer, prostate cancer, thyroid cancer, soft tissue sarcoma, brain cancer, cervical cancer, skin cancer, bone cancer, bile duct cancer, esophageal cancer, stomach cancer, testis cancer, head and neck cancer.

A further advantage of targeting cancer cells is that the nonsense mediate decay (NMD) mechanism for aberrant RNAs is typically reduces or inactivated in tumor cells (this is also common in for example virally infected cells).

It is well known that antisense oligonucleotides are preferentially taken up in the liver and kidney. In some embodiments the cell is a liver cell or a kidney cell.

However, for the use in the methods of the presently invention where the methods results in a gain of function (production of a peptide epitope), highly efficient uptake into the target cell may not be necessary, as long as sufficient oligonucleotide enters the cell so as to modulate the splicing event of the target RNA. In view of the amplification provided by the immune system, a modulation of splicing of, for example 0.5% or more may be effective in producing functional quantities of peptide epitope. The present methods are not therefore limited to use in liver or kidney cells, but may be generally applicable to any tissue or cell type. Furthermore, recent results from the inventors lab indicates that for splice switching oligonucleotides, as opposed to RNaseH recruiting gapmer, a very low cellular uptake of oligonucleotide can and often does result in a profound modulation of the splicing event.

In some embodiments the cell may be selected from the group consisting of a liver cell, a kidney cell, a mesenteric lymph node cell, a bone marrow cell, an immune cell, a monocyte cell, a macrophage cell, a T cell, a B-cell, a spleen cell, a uterine cell, an ovarian cell, a duodenum cell, a colon cell, an illium cell, a jejumum cell, a adopise cell, a lung cell, a muscle cell, a stomach cell, a pancreatic cell, a heart cell, a retinal cell, a brain cell, a neuronal cell, a dendritic cell, or a dorsal root ganglion cell.

The splice switching oligonucleotide may be administered to the cell via any suitable means, including for in vitro use, via gymnosis, transfection or electroporation. For in vivo use, the administration may be via systemic delivery or local delivery. In some embodiments the cell may be a tissue or cell which has been isolated from the subject, is then treatment by the method of the invention, prior to being re-introduced into the subject (ex-vivo administration).

### Cancer Target RNAs

The methods of the present invention are particularly useful in targeting cancer cells. Firstly, it is well known that the transcriptional control of several RNA transcripts are de-regulated in cancer cells—e.g. Cancer Genome Atlas TCGA.

In some embodiments the pre-mRNA is selected from the group consisting of CEMIP, ETV4, LRG5, NOX1, FOXP3, IGF2BP3, MAGE-A4, NY-ESO-1, EWSR1, FUS, and SS18.

### In Vivo and Therapeutic Methods and Uses

The invention provides for a method of immune modulating a target cell in a subject, said method comprising the steps of:

a. Vaccinate the subject with an agent comprising a peptide epitope, or encoding peptide epitope;
b. Administer a splice modulating oligonucleotide to the subject, wherein the splice modulating oligonucleotide targets a target RNA in a target cell in the subject, and modulates the splicing of the target RNA to produce an aberrant RNA transcript encoding an aberrant polypeptide containing the peptide epitope;
to trigger or enhance the immune response by the subject to the peptide epitope, such as a target cell expressing the peptide epitope;
wherein step a. and step b: may be in the order of step a. and then step b., or step b. and then step a., or step a. and step b. are performed simultaneously.

The invention provides for a method of immune modulating a target cell in a subject, said method comprising the steps of:

a. administer a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing a peptide epitope;
b. administer an antibody to the subject, wherein the antibody is specific for the peptide epitope
to trigger or enhance the immune response by the subject to the peptide epitope, such as a target cell expressing the peptide epitope; wherein step a. and step b: may be in the order of step a. and then step b., or step b. and then step a., or step a. and step b. are performed simultaneously.

The invention provides for a method of immune modulating a target cell in a subject, said method comprising the step of administering a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing the peptide epitope;

to trigger or enhance the immune response by the subject to the peptide epitope, such as a target cell expressing the peptide epitope.

The invention provides for a method of immunotherapy treatment of a disease in a subject, said method comprising the steps of

a. administer a splice modulating oligonucleotide to the subject, wherein the splice modulating oligonucleotide targets a target RNA in the target cell in the subject, and modulates the splicing of the target RNA to produce an aberrant RNA transcript encoding an aberrant polypeptide containing a peptide epitope;
b. administer an immunotherapy antibody to the subject, wherein the immunotherapy antibody is specific for the peptide epitope;
to trigger or enhance the immune response by the subject to the peptide epitope, such as the peptide epitope expressed by the target cell; wherein step a. and step b: may be in the order of step a. and then step b., or step b. and then step a., or step a. and step b. are performed simultaneously.

The invention provides method of immune modulating a target cell in a subject, said method comprising the administration of a splice modulating oligonucleotide to the subject, wherein the splice modulating oligonucleotide targets a RNA splice site or splice regulatory element in the target cell in the subject, and modulates the splicing of the RNA at the splice site or splice regulatory element to produce an aberrant mRNA transcript encoding an aberrant polypeptide containing the peptide epitope; wherein the aberrant epitope is immunogenic to the subject; to trigger or enhance the immune response by the subject to the target cell.

The invention provides a method of immune modulating a target cell in a subject, said method comprising the steps of:

a. Vaccinate the subject with an agent comprising a peptide epitope, or encoding a peptide epitope;
b. Administer a splice modulating oligonucleotide to the subject, wherein the splice modulating oligonucleotide targets a RNA splice site or splice regulatory element in the target cell in the subject, and modulates the splicing of the RNA at the splice site or splice regulatory element to produce an aberrant mRNA transcript encoding an aberrant polypeptide containing the peptide epitope;
to trigger or enhance the immune response by the subject to the target cell;
wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response.

In some embodiments, between steps a and b or b and a, an optionally waiting step c. may be employed, to e.g. allow the subject to develop an adaptive immune response to the antigen peptide (order of steps a, c, b), or to allow the expression of the epitope peptide on the target cell (order of steps b, c, a).

The invention provides for a method of immune modulating a target cell in a subject, said method comprising the steps of:
a. administer a splice modulating oligonucleotide to the subject, wherein the splice modulating oligonucleotide targets a RNA splice site or splice regulatory element in the target cell in the subject, and modulates the splicing of the RNA at the splice site or splice regulatory element to produce an aberrant RNA transcript encoding an aberrant polypeptide containing the peptide epitope;
b. administer an antibody to the subject, wherein the antibody is specific for peptide epitope
   to trigger or enhance the immune response by the subject to the target cell, wherein step a. and step b: may be in the order of step a. and then step b., or step b. and then step a., or step a. and step b. are performed simultaneously.

The method results in the expression or enhanced expression of the peptide epitope in the target cell, resulting in the triggering or enhanced immune response particularly when the antibody is administered in step b.

A waiting step c. may be performed between steps a and b, for example to allow for the expression of the peptide epitope in the target cell (order of steps a, b, c).

The invention provides for the use of a splice-modulating oligonucleotide for the production of a peptide epitope in a cell.

The invention provides for the use of a splice switching oligonucleotide in the immunotherapy treatment, e.g. of cancer, wherein the splice switching oligonucleotide targets a RNA to produce an aberrant RNA transcript encoding an aberrant polypeptide containing the peptide epitope, in the cell e.g. in the cancer cell; wherein the immunotherapy treatment comprises the administration of an therapeutic antibody which recognizes the peptide epitope to the subject.

The invention provides for the use of a splice switching oligonucleotide in the cancer vaccine therapy, wherein the splice switching oligonucleotide targets a RNA to produce an aberrant RNA transcript encoding an aberrant polypeptide containing the peptide epitope in a cancer cell wherein the vaccine therapy results in the generation of, or enhances the immune response, by the subject to the peptide epitope.

### Immunotherapy

Immunotherapy is a treatment which uses and enhances the subjects (patients) own adaptive immune system to treat disease, and is widely used in cancer treatment, wherein it is referred to as cancer immunotherapy. In some embodiments, the immunotherapy uses an antibody therapeutic, which may for example be the an antibody specific for the peptide epitope, or may for example be an antibody which enhances the subjects immune response to the disease, e.g. cancer.

### Vaccines & Vaccination

Vaccinate means to treat with a vaccine to produce immunity against a disease. Vaccination results in the activation of the adaptive immune system to an antigen which may be present in the vaccine or may be encoded in a nucleic acid present in the vaccine (such as in the form of a DNA, RNA or viral vaccine—collectively referred to herein as nucleic acid vaccine). In some embodiments the vaccine comprises the peptide epitope, or a nucleic acid vaccine encoding the peptide epitope. The delivery of a nucleic acid vaccine to a subject results in the expression of the peptide epitope in the subject, and thereby results in an immune response to the peptide epitope. Vaccines often comprise adjuvants which enhance the development of immunity by the subject.

In some embodiments the vaccine is used to treat cancer in the subject such as the cancer cell. Such vaccines are referred to as cancer vaccine.

### Pharmaceutical Salt

The splice modulating oligonucelotides used in the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention utilises a pharmaceutically acceptable salt of the antisense oligonucleotide or a conjugate thereof. In a preferred embodiment, the pharmaceutically acceptable salt is a sodium or a potassium salt.

### Pharmaceutical Composition

In a further aspect, the invention uses a pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide of the invention is administered at a dose of 10-1000 μg.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

An advantageous formulation of the splice modulating oligonucleotide is an exosome formulation.

Oligonucleotides or oligonucleotide conjugates may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety may in some embodiments be cleaved off the oligonucleotide once the prodrug is delivered to the site of action, e.g. the target cell.

### Administration of the Oligonucleotide

The oligonucleotides or pharmaceutical compositions used in the present invention may be administered topical or enteral or parenteral (such as, intravenous, subcutaneous, intramuscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

In some embodiments the oligonucleotide is administered in an exosome formulation.

### Exosomes

Exosomes are natural biological nanovesicles, typically in the range of 30 to 500 nm, that are involved in cell-cell communication via the functionally-active cargo (such as miRNA, mRNA, DNA and proteins).

Exosomes are secreted by all types of cells and are also found abundantly in the body fluids such as: saliva, blood, urine and milk. The major role of exosomes is to carry the information by delivering various effectors or signaling molecules between specific cells (Acta Pol Pharm. 2014 July-August; 71(4):537-43). Such effectors or signaling molecules can for example be proteins, miRNAs or mRNAs. Exosomes are currently being explored as a delivery vehicle for various drug molecules including RNA therapeutic molecules, to expand the therapeutic and diagnostic applications of such molecules. There are disclosures in the art of exosomes loaded with synthetic molecules such as siRNA, antisense oligonucleotides and small molecules which suggest or show advantages in terms of delivery and efficacy of such molecules compared to the free drug molecules (see for example Andaloussi et al 2013 Advanced Drug Delivery Reviews 65: 391-397, WO2014/168548, WO2016/172598, WO2017/173034 and WO 2018/102397).

Exosomes may be isolated from biological sources, such as milk (milk exosomes), in particular bovine milk is a abundant source for isolating bovine milk exosomes. See for example Manca et al., Scientific Reports (2018) 8:11321.

In some embodiments of the invention, the splice modulating oligonucleotide is encapsulated in an exosome (exosome formulation), examples of loading an exosome with a single stranded antisense oligonucleotide are described in EP application No. 18192614.8. In the methods of the invention the splice modulating oligonucleotide may be administered to the cell or to the subject in the form of an exosome formulation, in particular oral administration of the exosome formulations are envisioned.

In some embodiments, the splice modulating oligonucleotide may be conjugated, e.g. with a lipophilic conjugate such as cholesterol, which may be covalently attached to the splice modulating oligonucleotide via a biocleavable linker (e.g. a region of phosphodiester linked DNA nucleotides). Such lipophilic conjugates can facilitate formulation of splice modulating oligonucleotide into exosomes and may further enhance the delivery to the target cell.

### Check Point Inhibitors

Therapeutically approved immune check point inhibitors, which may be used in the therapeutic methods and uses of the invention include for example

| Name | Target | Approved |
|---|---|---|
| Ipilimumab | CTLA-4 | 2011 |
| Nivolumab | PD-1 | 2014 |
| Pembrolizumab | PD-1 | 2014 |
| Atezolizumab | PD-L1 | 2016 |
| Avelumab | PD-L1 | 2017 |
| Durvalumab | PD-L1 | 2017 |

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### Administration

The oligonucleotides or pharmaceutical compositions of the present invention may be administered topical or enteral or parenteral (such as, intravenous, subcutaneous, intramuscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

### Splice Modulating Embodiments

The following embodiments of the invention relate to splice modulating aspects of the invention.

1. A method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope.
2. The method according to embodiment 1, wherein either:
a) the splice modulating oligonucleotide modulates the of splicing of the target RNA, such as a pre-mRNA, to produce an aberrant RNA transcript introduced by the modulated splicing event, wherein the aberrant RNA (such as mRNA) transcript encodes an internal polypeptide deletion, to produce an aberrant polypeptide comprising an aberrant peptide sequence at the modulated splicing event (e.g. by skipping one or more exons), to produce the peptide epitope; and/or
b) the splice modulating oligonucleotide modulates the splicing of the target RNA, such as a pre-mRNA, to produce an aberrant RNA transcript (such as a mRNA) introduced by the modulated splicing event, wherein the aberrant RNA transcript encodes one or more codons from an intronic region of the target RNA, to produce an aberrant polypeptide comprising an aberrant peptide sequence which includes at least one or more peptide(s) encoded by the one or more codons originating from the intronic region, to produce the peptide epitope; and/or
c) the splice modulating oligonucleotide modulates the of splicing of the target RNA, such as a pre-mRNA, to produce an aberrant RNA transcript comprising a codon frame shift introduced by the modulated splicing event, wherein the aberrant RNA transcript produces a polypeptide with a C-terminal region of at least 1 amino acid, which is transcribed from the region of the aberrant RNA transcript at or 3′ to the codon frame shift.
   3. The method according to any one of embodiments 1 or 2, wherein the cell is a cancer cell, such as a tumor, lung cancer, breast cancer, colon cancer cell, mestastizised colon cancer cell, or a mestastizised colon cancer cell in the liver.
   4. The method according to any one of embodiments 1-3, wherein the method is an in vitro method or an in vivo method.
   5. The method according to any one of embodiments 1-4, wherein the target RNA is a RNA which is over-expressed in the cancer cell.
   6. The method according to any one of embodiments 1-5, wherein the peptide epitope is secreted from the cell.
   7. The method according to any one of embodiments 1-6, wherein the peptide epitope is presented on the cell as a MHC Class I or II molecule.
   8. The method according to any one of embodiments 1-7, wherein the polypeptide containing the peptide epitope further comprises a membrane binding domain.
   9. The method according to any one of embodiment 1-7, wherein the RNA is a pre-mRNA, such as a (e.g. human) pre-mRNA is selected from the group consisting of: CEMIP, ETV4, LRG5, NOX1, FOXP3, IGF2BP3, MAGE-A4, NY-ESO-1, EWSR1, FUS, PARPBP and SS18.
   10. The method according to any one of embodiments 1-7, wherein either:
a) the pre-mRNA is CEMIP, wherein the antisense oligonucleotide comprises a contiguous nucleotide sequence of at least 10 nucleotides, such as at least 12 nucleotides, which have 100% identity with a sequence selected from 1-82, or 193-274.
b) the pre-mRNA is ETV4, wherein the antisense oligonucleotide comprises a contiguous nucleotide sequence of at least 10 nucleotides, such as at least 12 nucleotides, which have 100% identity with a sequence selected from 83-164.
   11. The method according to any one of embodiments 1-10, wherein the splice modulating oligonucleotide comprises 2′ sugar modified nucleosides, such as 2′ sugar modified nucleosides independently selected from 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside, and LNA nucleosides.
   12. The method according to any one of embodiments 1-11, wherein the splice modulating oligonucleotide comprises modified internucleoside linkages, such as phosphorothioate internucleoside linkages.
   13. The method according to any one of embodiments 1-12, wherein the splice modulating oligonucleotide is a 2′-O-MOE oligonucleotide.
   14. The method according to any one of embodiments 1-13, wherein the splice modulating oligonucleotide is a LNA oligonucleotide such as a LNA mixmer.
   15. The method according to any one of embodiments 1-10, wherein the splice modulating oligonucleotide is a morpholino oligonucleotide.
   16. A method of immune modulating a target cell in a subject, said method comprising the steps of:
a. Vaccinate the subject with an agent comprising a peptide epitope, or encoding peptide epitope;
b. Administer a splice modulating oligonucleotide to the subject, wherein the splice modulating oligonucleotide targets a target RNA in a target cell in the subject, and modulates the splicing of the target RNA to produce an aberrant RNA transcript encoding an aberrant polypeptide containing the peptide epitope;
   to trigger or enhance the immune response by the subject to the peptide epitope, such as a target cell expressing the peptide epitope;
   wherein step a. and step b: may be in the order of step a. and then step b., or step b. and then step a., or step a. and step b. are performed simultaneously.
   17. A method of immune modulating a target cell in a subject, said method comprising the steps of:
a. administer a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing a peptide epitope;
b. administer an antibody to the subject, wherein the antibody is specific for the peptide epitope
   to trigger or enhance the immune response by the subject to the peptide epitope, such as a target cell expressing the peptide epitope; wherein step a. and step b: may be in the order of step a. and then step b., or step b. and then step a., or step a. and step b. are performed simultaneously.
   18. A method of immune modulating a target cell in a subject, said method comprising the step of administering a splice modulating oligonucleotide to the subject, wherein the splice modulating oligonucleotide targets a target RNA in a target cell in the subject, and modulates the splicing of the RNA to produce an aberrant RNA transcript encoding an aberrant polypeptide containing the peptide epitope;
   to trigger or enhance the immune response by the subject to the peptide epitope, such as a target cell expressing the peptide epitope.
   19. The method according to any one of embodiments 16-18, wherein the method further comprises the step of administering a check point inhibitor to the subject, such as a PDL1 inhibitor, a PD1 inhibitor or CTLA-4 inhibitor.
   20. A method of immunotherapy treatment of a disease in a subject, said method comprising the steps of
a. administer a splice modulating oligonucleotide to the subject, wherein the splice modulating oligonucleotide targets a target RNA in the target cell in the subject, and modulates the splicing of the target RNA to produce an aberrant RNA transcript encoding an aberrant polypeptide containing a peptide epitope;
b. administer an immunotherapy antibody to the subject, wherein the immunotherapy antibody is specific for the peptide epitope;
   to trigger or enhance the immune response by the subject to the peptide epitope, such as the peptide epitope expressed by the target cell; wherein step a. and step b: may be in the order of step a. and then step b., or step b. and then step a., or step a. and step b. are performed simultaneously.
   21. The method according to any one of embodiments 16-20, wherein the method is a method of treatment for cancer.
   22. The method according to any one of embodiments 16-21, wherein the cell is a cancer cell, such as a tumor, lung cancer, breast cancer, colon cancer cell, mestastizised colon cancer cell, or a mestastizised colon cancer cell in the liver.
   23. The method according to any one of embodiments 16-22, wherein step a) comprises the method according to any one of embodiments 1-15; or the method according to any one of embodiments 16, or embodiments 20 or 21 when dependent upon embodiment 16, wherein step c. comprises the method according to any one of embodiments 1-15.
   24. Use of a splice-modulating oligonucleotide for the production of a peptide epitope in a cell.
   25. Use of a splice modulating oligonucleotide in the immunotherapy treatment of cancer, wherein the splice modulating oligonucleotide targets a RNA to produce an aberrant RNA transcript encoding an aberrant polypeptide containing the peptide epitope in a cancer cell wherein the immunotherapy treatment comprises the administration of an therapeutic antibody which recognizes the peptide epitope.
   26. Use of a splice modulating oligonucleotide in the cancer vaccine therapy, wherein the splice modulating oligonucleotide targets a RNA to produce an aberrant RNA transcript encoding an aberrant polypeptide containing the peptide epitope wherein the vaccine therapy results in the generation of or enhances the immune response by the subject to the peptide epitope.
   27. An antisense oligonucleotide capable of modulating the splicing of CEMIP pre-mRNA, wherein the antisense oligonucleotide comprises a contiguous nucleotide sequence of at least 10 nucleotides, such as at least 12 nucleotides, which have 100% identity with a sequence selected from SEQ ID NO 1-82, or 193-274.
   28. An antisense oligonucleotide capable of modulating the splicing of ETV4 pre-mRNA, wherein the antisense oligonucleotide comprises a contiguous nucleotide sequence of at least 10 nucleotides, such as at least 12 nucleotides, which have 100% identity with a sequence selected from SEQ ID NO 83-164.
   29. An splice modulating antisense oligonucleotide comprising or consisting the sequence 1-164, or a compound selected from the group consisting of O1-O164, or O165-O246.
   30. A vaccine or immunotherapy agent which comprises the peptide epitope, such as a peptide epitope selected from the groups 188, 189, 190, 191 or 192.
   31. A polypeptide which is or comprises the peptide, such as a peptide selected from the groups 188, 189, 190, 191 or 192.
   32. A polypeptide which is or comprises the peptide, such as a peptide selected from the groups 188, 189, 190, 191 or 192, for use in medicine, such as for use as a vaccine or immunotherapy agent.

### EXAMPLES

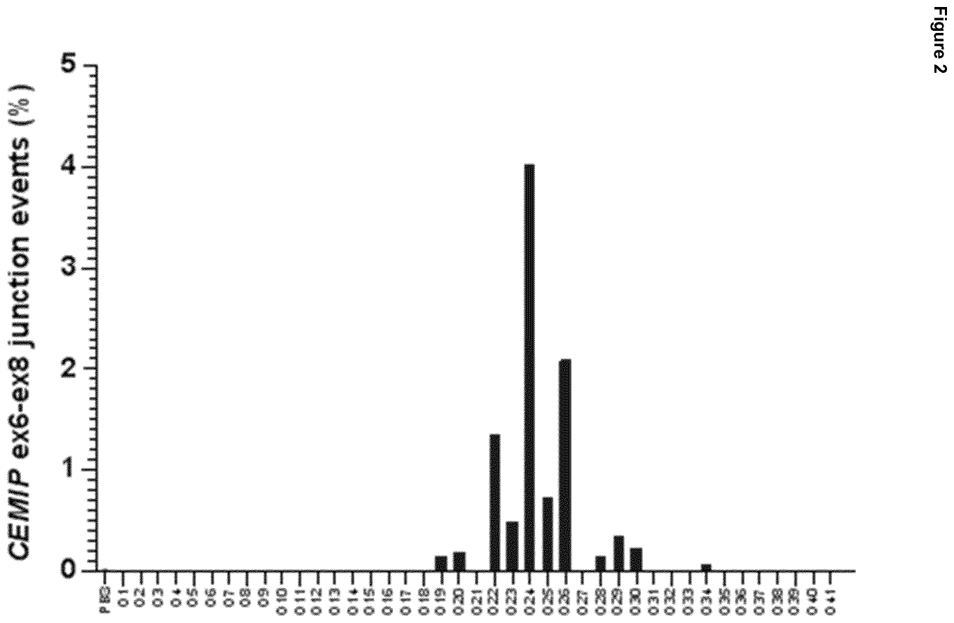

### Example 1. A Novel Splice Junction Between Exon 6 and Exon 8 in CEMIP mRNA is Induced by Specific Oligonucleotides. (Results Shown in FIG. 2)

To induce a novel splice junction between exon 6 and exon 8 based on the transcript isoform CEMIP-201 transcript (ENST00000220244.7), we designed oligonucleotides to target the CEMIP exon 7 sequence.

4×10³ Colo-205 cells were seeded in 96-well plate format and cultured in RPMI media supplemented with 10% FBS and 1% pen/strep. 41 different oligonucleotides targeting CEMIP pre-mRNA sequence (or vehicle only, PBS) were added to the cells at 25 μM final concentration. After 4 days, cells were harvested, and RNA was extracted using RNeasy Mini extraction kit (Qiagen). cDNA was generated using iScript Advanced cDNA synthesis kit and processed for ddPCR analysis (Biorad). The expression level of CEMIP mRNA containing the induced exon 6/exon 8 splice junction is represented as the percentage of total CEMIP transcripts. QuantaSoft software (Biorad) was used for analysis. The following probe based assays were used to detect the splicing event: CEMIP exon 6/exon 8 junction (forward primer GCGATGACCAAATTGGGAAG (SEQ ID NO 167), reverse primer GCCATGCTCTGTCTGGAA (SEQ ID NO 168), probe/56-FAM/CACCTTGGA/ZEN/TTTAGGACATCGAGGCTC/3IABkFQ/-(SEQ ID NO 169)); total CEMIP (forward primer CTCGGTGCTGAGGTTAACTC (SEQ ID NO 170), reverse primer TCAGACTAAAGGTGGGGAGAA (SEQ ID NO 171), probe /5HEX/TCAGACCTC/ZEN/TGGAAAGCTCACCCA/3IABkFQ/SEQ ID NO 172). Compared to the wild-type CEMIP mRNA, the induced exon 6/exon 8 junction generates a frame shift in the mRNA coding sequence with a subsequent novel 28 aminoacid-long polypeptide at CEMIP C-terminal region (R T S R L C C C P G I Q I V P D R A W R I F Q C F F V Q, SEQ ID NO 188).

The following LNA containing oligonucleotides were used to induce alteration in splicing events. All internucleoside linkages are phopshorothionate. Upper and lower case indicate LNA and DNA nucleobases, respectively.

| | | |
|---|---|---|
| O1 | GAaAaACaTAacAA | SEQ ID NO 1 |
| O2 | AGaAAaACaTAaCA | SEQ ID NO 2 |
| O3 | AaGAaAAaCAtaAC | SEQ ID NO 3 |
| O4 | AAaGAaAAaCAtAA | SEQ ID NO 4 |
| O5 | AAaAGaAAaACaTA | SEQ ID NO 5 |
| O6 | AAaAaGAaaAAcAT | SEQ ID NO 6 |
| O7 | AAaAAaGAaAAaCA | SEQ ID NO 7 |
| O8 | AAaAAaAGaAAaAC | SEQ ID NO 8 |
| O9 | GAaAAaAaGAaaAA | SEQ ID NO 9 |
| O10 | TGaAAaAAaGAaAA | SEQ ID NO 10 |
| O11 | CTgAAaAAaAGaAA | SEQ ID NO 11 |
| O12 | TCtGAaAAaAAgAA | SEQ ID NO 12 |
| O13 | GTcTGaAAaAAaGA | SEQ ID NO 13 |
| O14 | TGtCTgAAaAAaAG | SEQ ID NO 14 |
| O15 | GTgTCtGAaAAaAA | SEQ ID NO 15 |
| O16 | GGtGTcTGaAAaAA | SEQ ID NO 16 |
| O17 | GGgTGtCTgAAaAA | SEQ ID NO 17 |
| O18 | AGgGTgTCtGAaAA | SEQ ID NO 18 |
| O19 | AaGGgTGtCTgaAA | SEQ ID NO 19 |
| O20 | CaaGGgTGtCTgAA | SEQ ID NO 20 |
| O21 | CAaGGgTGtCTgAA | SEQ ID NO 21 |
| O22 | CCaaGgGtGTctGA | SEQ ID NO 22 |
| O23 | CCaaGgGTgTctGA | SEQ ID NO 23 |
| O24 | CCaAGgGtGTctGA | SEQ ID NO 24 |
| O25 | TccAaGGgtGTcTG | SEQ ID NO 25 |
| O26 | TccAAgGGtGTcTG | SEQ ID NO 26 |
| O27 | TcCAaGgGTgTcTG | SEQ ID NO 27 |
| O28 | CTccAaGgGTgtCT | SEQ ID NO 28 |
| O29 | CtcCAaGgGTgtCT | SEQ ID NO 29 |
| O30 | CTcCAaGgGTgtCT | SEQ ID NO 30 |
| O31 | GGGTGTCT | SEQ ID NO 31 |
| O32 | ActCcAaGgGTgTC | SEQ ID NO 32 |
| O33 | ACtcCaaGgGTgTC | SEQ ID NO 33 |
| O34 | ACtcCAaGgGTgTC | SEQ ID NO 34 |
| O35 | AActCCaAGgGtGT | SEQ ID NO 35 |
| O36 | AaCtCCaAGgGtGT | SEQ ID NO 36 |
| O37 | AaCtCCaAgGGtGT | SEQ ID NO 37 |
| O38 | AAaCTcCAaGGgTG | SEQ ID NO 38 |
| O39 | AAaACtCCaAGgGT | SEQ ID NO 39 |
| O40 | GAaaACtCCaAgGG | SEQ ID NO 40 |
| O41 | AGaAAaCTcCAaGG | SEQ ID NO 41 |

### Example 2. A Novel Splice Junction Between Exon 27 and Exon 29 in CEMIP mRNA is Induced by Specific Oligonucleotides. (Results Shown in FIG. 3)

To induce a novel splice junction between exon 27 and exon 29 based on the transcript isoform CEMIP-201 transcript (ENST00000220244.7), we designed oligonucleotides to target the CEMIP pre-mRNA at sites flanking or overlapping CEMIP exon 28 sequence:

catgcccactttctccagtttgctctctccctctggtctaattggtttcttctcacacagTTCCATAGTGCTTATGGCATCA AAGGGAAGATACGTCTCCAGAGGCCCATGGACCAGAGTGCTGGAAAAGCTTGGGGCA GACAGGGGTCTCAAGTTGAAAGgtaagggtttgaactggggtttaaactgaccccaaaaccagagaaggcaaa tgc (SEQ ID NO 297, Exon 28 nucleotides in capital letters, lower case letter are flanking intronic sequences).

4×10³ Colo-205 cells were seeded in 96-well plate format and cultured in RPMI media supplemented with 10% FBS and 1% pen/strep. 41 different oligonucleotides targeting CEMIP pre-mRNA sequence (or vehicle only, PBS) were added to the cells at 25 μM final concentration. After 4 days, cells were harvested, and RNA was extracted using RNeasy Mini extraction kit (Qiagen). cDNA was generated using iScript Advanced cDNA synthesis kit and processed for ddPCR analysis (Biorad). The expression level of CEMIP mRNA containing the induced exon 27/exon 29 splice junction is represented as the percentage of total CEMIP transcripts. QuantaSoft software (Biorad) was used for analysis. The following probe based assays were used to detect the splice event: CEMIP exon 27/exon 29 junction (forward primer GGAACTCCATTCTGCAAGG (SEQ ID NO 173), reverse primer CCTCAGTGTCCAGTGTCA (SEQ ID NO 174), /56-FAM/CCA TCCCTG/ZEN/ACAAAGCAAATGGCATTC/3IABkFQ/(SEQ ID NO 175)); total CEMIP (forward primer CTCGGTGCTGAGGTTAACTC (SEQ ID NO 176), reverse primer TCAGACTAAAGGTGGGGAGAA (SEQ ID NO 177), probe /5HEX/TCAGACCTC/ZEN/TGGAAAGCTCACCCA/3IABkFQ/(SEQ ID NO 172)). Compared to the wild-type CEMIP mRNA, the induced exon 27/exon 29 junction generates a frame shift in the mRNA coding sequence with a subsequent novel 21 aminoacid-long polypeptide at CEMIP C-terminal region (K A N G I R W L Q R Q L P A H L G D T G H, SEQ ID NO 189). The following LNA containing oligonucleotides were used to induce alteration in splicing events. All internucleoside linkages are phopshorothionate. Upper and lower case indicate LNA and DNA nucleobases, respectively.

| | | |
|---|---|---|
| O42 | AGaAaCCaaTTaGA | SEQ ID NO 42 |
| O43 | AaGAaaCCaATtAG | SEQ ID NO 43 |
| O44 | GAaGAaaCCaAtTA | SEQ ID NO 44 |
| O45 | AGaAGaAaCCaaTT | SEQ ID NO 45 |
| O46 | GAgAaGAaaCCaAT | SEQ ID NO 46 |
| O47 | TGaGAaGAaACcAA | SEQ ID NO 47 |
| O48 | GTgAGaAGaAAcCA | SEQ ID NO 48 |
| O49 | TgTGaGAaGAaaCC | SEQ ID NO 49 |
| O50 | GTgTGaGAaGAaAC | SEQ ID NO 50 |
| O51 | TGtGTgAGaAGaAA | SEQ ID NO 51 |
| O52 | CTgTGtGAgAAgAA | SEQ ID NO 52 |
| O53 | AcTGtgTGaGAaGA | SEQ ID NO 53 |
| O54 | AaCTgTGtgAGaAG | SEQ ID NO 54 |
| O55 | GAaCTgTGtGAgAA | SEQ ID NO 55 |
| O56 | GGaaCTgTgTgaGA | SEQ ID NO 56 |
| O57 | TGgAaCTgtGTgAG | SEQ ID NO 57 |
| O58 | ATgGAaCTgTGtGA | SEQ ID NO 58 |
| O59 | TAtgGAaCTgTgTG | SEQ ID NO 59 |
| O60 | TAtGGaACtGTgTG | SEQ ID NO 60 |
| O61 | CTaTGgAAcTGtGT | SEQ ID NO 61 |
| O62 | ACtATgGAaCTgTG | SEQ ID NO 62 |
| O63 | GAACTGTG | SEQ ID NO 63 |
| O64 | CAcTAtGGaACtGT | SEQ ID NO 64 |
| O65 | GGAACTGT | SEQ ID NO 65 |
| O66 | GCaCtATgGAacTG | SEQ ID NO 66 |
| O67 | GCaCTaTgGAacTG | SEQ ID NO 67 |
| O68 | GCaCTaTGgAAcTG | SEQ ID NO 68 |
| O69 | TGGAACTG | SEQ ID NO 69 |
| O70 | AGcaCTaTgGAaCT | SEQ ID NO 70 |
| O71 | AGcaCTaTGgAaCT | SEQ ID NO 71 |
| O72 | ATGGAACT | SEQ ID NO 72 |
| O73 | AaGCaCTaTGgaAC | SEQ ID NO 73 |
| O74 | TATGGAAC | SEQ ID NO 74 |
| O75 | TAaGCaCTaTGgAA | SEQ ID NO 75 |
| O76 | CTATGGAA | SEQ ID NO 76 |
| O77 | ATaaGCaCTaTgGA | SEQ ID NO 77 |
| O78 | ACTATGGA | SEQ ID NO 78 |
| O79 | CaTAaGCaCTatGG | SEQ ID NO 79 |
| O80 | CACTATGG | SEQ ID NO 80 |
| O81 | CCaTAaGcaCTaTG | SEQ ID NO 81 |
| O82 | GCcATaaGCaCtAT | SEQ ID NO 82 |

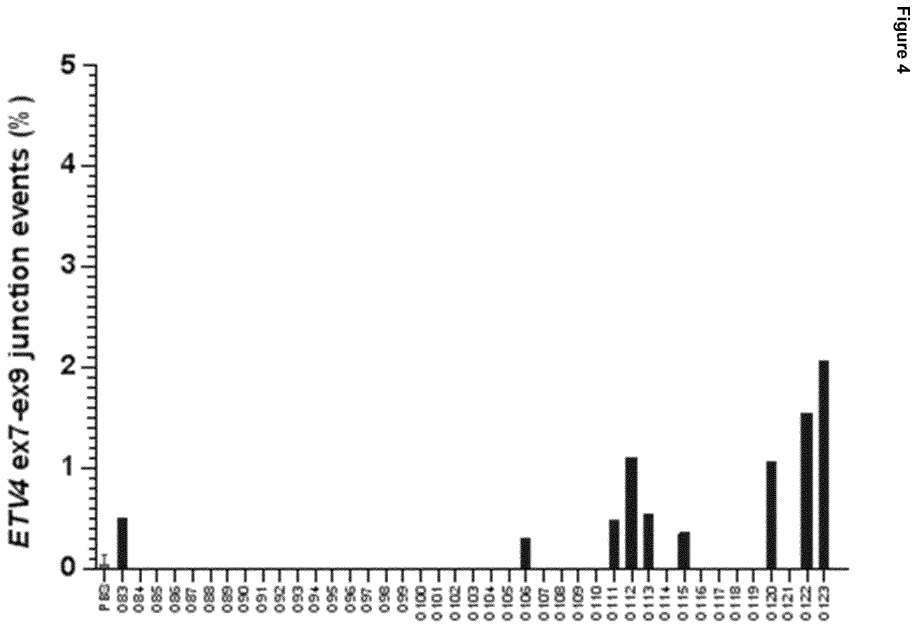

### Example 3. A Novel Splice Junction Between Exon 7 and Exon 9 in ETV4 mRNA is Induced by Specific Oligonucleotides. (Results Shown in FIG. 4)

4×10³ Colo-205 cells were seeded in 96-well plate format and cultured in RPMI media supplemented with 10% FBS and 1% pen/strep. 41 different oligonucleotides targeting ETV4 pre-mRNA sequence (or vehicle only, PBS) were added to the cells at 25 μM final concentration. After 4 days, cells were harvested, and RNA was extracted using RNeasy Mini extraction kit (Qiagen). cDNA was generated using iScript Advanced cDNA synthesis kit and processed for ddPCR analysis (Biorad). The expression level of ETV4 mRNA containing the induced exon 7/exon 9 splice junction is represented as the percentage of total ETV4 transcripts. QuantaSoft software (Biorad) was used for analysis. The following probe based assays were used to detect the splice event: ETV4 exon 7/exon 9 junction: (forward primer GGTGATCAAACAGGAACAGAC, reverse primer GGGACAACGCAGACATC SEQ ID NO 178, /56-FAM/CCTACGACT/ZEN/CAGGCTATGGCTATGAG/3IABkFQ/(SEQ ID NO 179)); total ETV4 (forward primer CGCTCGCTCCGATACTATTATG (SEQ ID NO 180), reverse primer CAAACTCAGCCTTGAGAGCTG (SEQ ID NO 181), probe /5HEX/CATCATGCA/ZEN/GAAGGTGGCTGGTGA/3IABkFQ/(SEQ ID NO 182)). Compared to the wild-type ETV4 mRNA, the induced exon 7/exon 9 junction generated an in-frame deletion of 25 aminoacid-long polypeptide coded by exon 8, which in turn produces a novel polypeptidic junction within ETV4 protein. (Q E Q T D F A Y D S G Y G Y E K P L R P, SEQ ID NO 190, underlined is indicated the new junction site).

The following LNA containing oligonucleotides were used to induce alteration in splicing events. All internucleoside linkages are phopshorothionate. Upper and lower case indicate LNA and DNA nucleobases, respectively.

| | | |
|---|---|---|
| O83 | GgAaGAaGGgGtGA | SEQ ID NO 83 |
| O84 | GgGAagAaGGggTG | SEQ ID NO 84 |
| O85 | GggGAagAaGGgGT | SEQ ID NO 85 |
| O86 | GgGggAaGAaGgGG | SEQ ID NO 86 |
| O87 | TgGGgGAagAagGG | SEQ ID NO 87 |
| O88 | GTgGggGAaGAaGG | SEQ ID NO 88 |
| O89 | TgTGgGGgaAgaAG | SEQ ID NO 89 |
| O90 | CTgTGgGGgAagAA | SEQ ID NO 90 |
| O91 | TcTgTggGgGAaGA | SEQ ID NO 91 |
| O92 | AtCTgtGGgGgaAG | SEQ ID NO 92 |
| O93 | CAtCTgTgGgGgAA | SEQ ID NO 93 |
| O94 | ACatCTgtGgGgGA | SEQ ID NO 94 |
| O95 | GacATctGTgGgGG | SEQ ID NO 95 |
| O96 | TgACatCTgTggGG | SEQ ID NO 96 |
| O97 | GTgAcATctGTgGG | SEQ ID NO 97 |
| O98 | GTgaCatCTgTgGG | SEQ ID NO 98 |
| O99 | GTgaCAtCTgTgGG | SEQ ID NO 99 |
| O100 | GgTgACatCTgtGG | SEQ ID NO 10O |
| O101 | GgTgaCAtCTgtGG | SEQ ID NO 101 |
| O102 | GGtGAcAtCTgtGG | SEQ ID NO 102 |
| O103 | CgGTgACatCTgTG | SEQ ID NO 103 |
| O104 | CgGTgaCAtCTgTG | SEQ ID NO 104 |
| O105 | CGgTgaCAtCTgTG | SEQ ID NO 105 |
| O106 | CCgGTgAcATctGT | SEQ ID NO 106 |
| O107 | CCgGTgACaTctGT | SEQ ID NO 107 |
| O108 | CCgGTgaCAtCtGT | SEQ ID NO 108 |
| O109 | CCcgGTgAcATcTG | SEQ ID NO 109 |
| O110 | CCcGGtGAcATcTG | SEQ ID NO 110 |
| O111 | AccCgGTgaCAtCT | SEQ ID NO 111 |
| O112 | AcCCgGTgACatCT | SEQ ID NO 112 |
| O113 | AcCCgGTgaCAtCT | SEQ ID NO 113 |
| O114 | CAccCgGTgAcaTC | SEQ ID NO 114 |
| O115 | CAccCgGTgACaTC | SEQ ID NO 115 |
| O116 | CAcCCgGTgAcaTC | SEQ ID NO 116 |
| O117 | GcaCCcgGTgAcAT | SEQ ID NO 117 |
| O118 | GCacCCggTgAcAT | SEQ ID NO 118 |
| O119 | GCacCCgGTgAcAT | SEQ ID NO 119 |
| O120 | CGcAccCgGTgaCA | SEQ ID NO 120 |
| O121 | GcgCAccCgGTgAC | SEQ ID NO 121 |
| O122 | TgCGcACccGgtGA | SEQ ID NO 122 |
| O123 | ATgCGcACccGgTG | SEQ ID NO 123 |

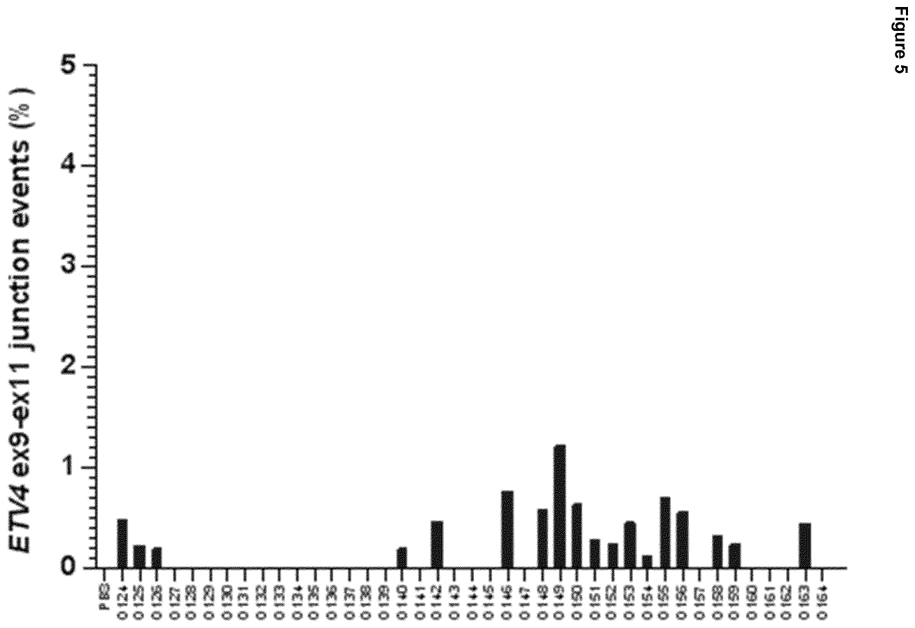

### Example 4. A Novel Splice Junction Between Exon 9 and Exon 11 in ETV4 mRNA is Induced by Specific Oligonucleotides. (Results Shown in FIG. 5)

4×10³ Colo-205 cells were seeded in 96-well plate format and cultured in RPMI media supplemented with 10% FBS and 1% pen/strep. 41 different oligonucleotides targeting ETV4 pre-mRNA sequence (or vehicle only, PBS) were added to the cells at 25 μM final concentration. After 4 days, cells were harvested, and RNA was extracted using RNeasy Mini extraction kit (Qiagen). cDNA was generated using iScript Advanced cDNA synthesis kit and processed for ddPCR analysis (Biorad). The expression level of ETV4 mRNA containing the induced exon 9/exon 11 splice junction is represented as the percentage of total ETV4 transcripts. QuantaSoft software (Biorad) was used for analysis. The following probe based assays were used to detect the splice event: ETV4 exon 9/exon 11 junction: (forward primer CTCTGCGACCATTCCCA (SEQ ID NO 183), reverse primer GCTCAGCTTGTCGTAATTCATG (SEQ ID NO 184), /56-FAM/TTG TCCCTG/ZEN/AGAGTCGCCAGG/3IABkFQ/(SEQ ID NO 298)); total ETV4 (forward primer CGCTCGCTCCGATACTATTATG (SEQ ID NO 185), reverse primer CAAACTCAGCCTTGAGAGCTG (SEQ ID NO 186), probe /5HEX/CATCATGCA/ZEN/GAAGGTGGCTGGTGA/3IABkFQ/(SEQ ID NO 187)). Compared to the wild-type ETV4 mRNA, the induced exon 9/exon 11 junction generates a frame-shift in the mRNA coding sequence with a subsequent novel 27 aminoacid-long polypeptide at ETV4 C-terminal region (G R Q A L G H P E E P A S H E L R Q A E P L A P I L L, SEQ ID NO 190).

The following LNA containing oligonucleotides were used to induce alteration in splicing events. All internucleoside linkages are phopshorothionate. Upper and lower case indicate LNA and DNA nucleobases, respectively.

| | | |
|---|---|---|
| O124 | AaCAcGAaaATaGG | SEQ ID NO 124 |
| O125 | GAaCAcGAaAAtAG | SEQ ID NO 125 |
| O126 | GGaACaCGaAAaTA | SEQ ID NO 126 |
| O127 | GGgAaCAcGAaaAT | SEQ ID NO 127 |
| O128 | GGgGAaCAcGAaAA | SEQ ID NO 128 |
| O129 | TGgGGaACaCGaAA | SEQ ID NO 129 |
| O130 | CtGGgGAaCAcgAA | SEQ ID NO 130 |
| O131 | CCtGGgGAacAcGA | SEQ ID NO 131 |
| O132 | TcCTgGGgAAcaCG | SEQ ID NO 132 |
| O133 | CTcCTgGgGAacAC | SEQ ID NO 133 |
| O134 | TcTccIgGgGAaCA | SEQ ID NO 134 |
| O135 | GtCTcCTggGgaAC | SEQ ID NO 135 |
| O136 | TgtCTccIgGGgAA | SEQ ID NO 136 |
| O137 | ATgtCTccTgGgGA | SEQ ID NO 137 |
| O138 | ATgtCTccTGggGA | SEQ ID NO 138 |
| O139 | ATgtCTcCTgGgGA | SEQ ID NO 139 |
| O140 | GAtGtcTccTggGG | SEQ ID NO 140 |
| O141 | GAtGTcTcaggGG | SEQ ID NO 141 |
| O142 | GatGtCTcCTggGG | SEQ ID NO 142 |
| O143 | TgATgtCTccTgGG | SEQ ID NO 143 |
| O144 | TgATgtCtcCTgGG | SEQ ID NO 144 |
| O145 | TgATgtCTcCTgGG | SEQ ID NO 145 |
| O146 | TTgATgtCtCctGG | SEQ ID NO 146 |
| O147 | TTgATgtCTcCtGG | SEQ ID NO 147 |
| O148 | TTgATgTctCCtGG | SEQ ID NO 148 |
| O149 | CTtGAtGTcTccTG | SEQ ID NO 149 |
| O150 | CTtgATgTCtCcTG | SEQ ID NO 150 |
| O151 | CTtGAtGTcTCcTG | SEQ ID NO 151 |
| O152 | GcTTgATgtCTcCT | SEQ ID NO 152 |
| O153 | GcTTgATgTCtcCT | SEQ ID NO 153 |
| O154 | GcTTgaTGtCTcCT | SEQ ID NO 154 |
| O155 | TgcTTgATgtCtCC | SEQ ID NO 155 |
| O156 | TgCTtgATgtCtCC | SEQ ID NO 156 |
| O157 | TgcTTgATgTCtCC | SEQ ID NO 157 |
| O158 | CTgCTtgATgTcTC | SEQ ID NO 158 |
| O159 | CTgCTtGAtgTcTC | SEQ ID NO 159 |
| O160 | CTgCTtGAtGTcTC | SEQ ID NO 160 |
| O161 | CCtgCTtgATgtCT | SEQ ID NO 161 |
| O162 | TcCTgcTTgATgTC | SEQ ID NO 162 |
| O163 | TtcCTgCTtGAtGT | SEQ ID NO 163 |
| O164 | CTtcCTgCtTgaTG | SEQ ID NO 164 |

### Example 5. Finding Candidate RNAs for the Generation of Engineered Neo-Epitopes

Candidate RNAs for the generation of engineered neo-epitopes will be identified by comparative analysis of gene expression data (such as RNA-seq and microarray profiling). In particular, by comparing the expression profiles of the diseased cells of interest with the profiles of normal tissues (and/or normal cells), a subset of transcripts (or isoforms)—which are exclusively or highly upregulated in the diseased cells—will be selected. A comprehensive analysis of all possible splice-switch events will be performed in silico to define which ones have the potential to generate novel epitopes upon exposure to splice-switch oligonucleotides. Examples of transcripts identified by this approach are: CEMIP (colon cancer), ETV4 (colon cancer), LRG5 (colon cancer), NOX1 (colon cancer), FOXP3 (T-REGS), IGF2BP3 (general cancer), MAGE-A4 (general cancer), NY-ESO-1 (general cancer), EWSR1 (sarcoma, aberrant splicing), FUS (sarcoma, aberrant splicing), SS18 (sarcoma, aberrant splicing).

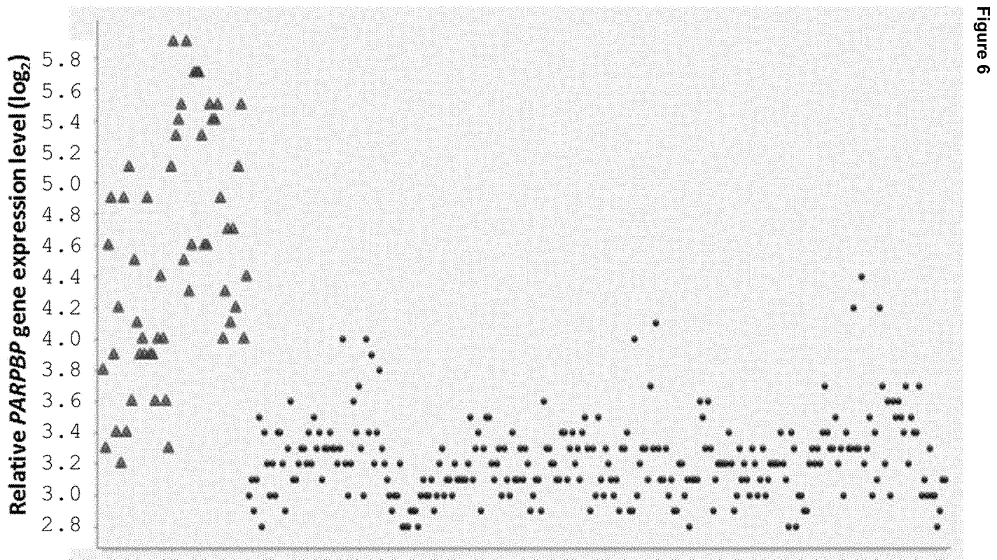

### Example 6. Finding Candidate RNAs for the Generation of Engineered Neo-Epitopes for Lung Squamous Cell Carcinoma. (Illustrated in FIGS. 6 & 7)

A two group comparison of the gene expression profiles (measured by Affymetrix U133 plus 2 arrays, 54,676 probes) was performed on 56 lung squamous cell carcinomas clinical samples against normal human tissues (Adrenal cortex gland, n=4; Bladder, n=3; Breast, n=30; Cervix, n=3; Colon, n=8; Esophagous, n=4; Kidney, n=37; Liver, n=22; Lung, n=6; Lung epithelium, n=21; Ovary, n=4; Pancreas, n=37; Prostate, n=24; Skeletal muscle, n=14; Skin, n=4; Small Intestine, n=3; Stomach, n=19; Testes, n=11; Thyroid, n=10. PARPBP resulted as one of the most differentially expressed genes (p-value=3.857e⁻⁶⁹). The expression data measured by the PARPBP probe 220060_s_at is depicted in the figure below. Pink triangles correspond to lung squamous cell carcinomas samples, blue dots represent normal tissues. In order to further validate the previous analysis, the expression of PARPBP was examined in the GTEX database (https://glexportal.org/home/gene/PARPBP). Consistent with the microarray data, PARPBP shows negligible expression in the majority of healthy human tissues, confirming its potential use as a target transcript for neo-antigen engineering.

PARPBP-201 ENST00000327680.6

| No. | Exon/Intron | Length | Sequence |
|---|---|---|---|
| | 5′ upstream | | ..........gacagcggcgactgcggcggccgcgggagggcatcccgttggggatc |
| | sequence | | ctt (SEQ ID NO 275) |
| 1 | ENSE000 | 42 | CCGCACACTGAAGAGTACGTCTTCGGGTCTACCCCTAATCAC |
| | 02301033 | | (SEQ ID NO 276) |
| | Intron 1-2 | 3,599 | gtaagtctcgcgtctgccctacctgccctgtcgcatttttaagtggtgattcaggct |
| | | | gtggcttttgagggtgttttgcactgtaatacctcggtcgtagcaaagtgccgtggg |
| | | | accgaagtattatggtgcagtgacttgacgctcgagcctggttgggcctgggggctg |
| | | | taggcggagttcacagtatctagcgatgg....................accaaagg |
| | | | ggagggaaatatgattgcgcagaaattgtattattttgttttcctaaatttaattaa |
| | | | aagcaatcttggtaaatatgaaatgacctctcattcactatatgtttttctctatct |
| | | | gtgcttgcctatacatgttaaatgttaatttcaggttaacataagatactttaactt |
| | | | tgtattctgtcctactttaag(SEQ ID NO 277) |
| 2 | ENSE000 | 156 | ATAATGGCTGTGTTTAATCAGAAGTCTGTCTCGGATATGATTAAAGAGTTTCGAAAA |
| | 03490031 | | AATTGGCGTGCTCTTTGTAACTCTGAGAGAACTACTCTATGTGGTGCAGACTCCATG |
| | | | CTCTTGGCATTGCAGCTTTCTATGGCGGAGAACAACAAACAG(SEQ ID NO 278) |
| | Intron 2-3 | 24,188 | gttggtaaactatatttgggttaacttgtttttttaaagcaaaatttcattgctgcc |
| | | | catttgaataaacttaagcttaagaatattaatttctttacattatgtgcccttctg |
| | | | tgataatacaccattttcacaaatgcctctgtgtatctgtgtcagaatgttatgata |
| | | | gaatttgaaatgaattatttcaaaattaa....................taaaatca |
| | | | tgaaaacctttttcttttatatctggtttccaaaattgtctcatagttcaacttggg |
| | | | tgttctcttcaaacccacaaaaattgaatgtagactttaccaaaatttaatttgtat |
| | | | ttcttatctcagttataagttattgaatattctggtacccaactttatttttttttt |
| | | | ttttggcattatctttttcag (SEQ ID NO 279) |
| 3 | ENSE000 | 234 | CACAGTGGAGAATTTACAGTCTCTCTCAGTGATGTTTTATTGACATGGAAATACTTG |
| | 03747564 | | CTCCATGAGAAATTGAACTTACCAGTTGAAAACATGGACGTGACTGACCATTATGAG |
| | | | GACGTTAGGAAGATTTATGATGATTTCTTGAAGAACAGTAATATGTTAGATCTGATT |
| | | | GATGTTTATCAAAAATGTAGGGCTTTGACTTCTAATTGTGAAAATTATAACACAGTA |
| | | | TCTCCT(SEQ ID NO 280) |
| | Intron 3-4 | 5,405 | gtaagtattttttaaacaattctattttaatcaaattaaaatttagctctatttatt |
| | | | ttgaattatagtatcaccagttatagtaacttggtaatgtttttgctttgtgaatca |
| | | | tgttaaaaatgggaaaatttagctacctgaaagctacttattacttgaaagaaagat |
| | | | tttgtggtaaattttcttggacttgtgtt....................gtctttat |
| | | | ttgtgtagcacacaggaagaacccatgggcaattacattatgaagtttaattatatg |
| | | | tattcttaatgattgctaacaactgtcaggtaatactctcaactgaaacatcaatga |
| | | | cactgagtagagctttatgtgaagtattttttatagtcagcatagcattttattagt |
| | | | aatactctatgttttctacag(SEQ ID NO 281) |
| 4 | ENSE000 | 108 | AGTCAACTACTGGATTTTCTGTCTGGCAAACAGTATGCAGTAGGTGATGAAACTGAT |
| | 03595314 | | CTTTCTATACCAACATCACCAACAAGTAAATACAACCGTGATAATGAAAAG |
| | | | (SEQ ID NO 282) |
| | Intron 4-5 | 10,461 | gtactgataaactgtgagtaaattaaagcagactataatccctttcaaatcactgaa |
| | | | tttggtggtaccttaaagttactaatgctttgtaaaaatttttttaacaaatcttta |
| | | | gtatgtttaaaactgtattcgcaaagaataatacattggaggctggtgagaatgtga |
| | | | taaaagagtctttatggtttggggccaag....................tttggtag |
| | | | tcagtcaagaatttggggagagatggagtttattgattgcccaacctgtttttatat |
| | | | ggcagttttatttttctaaagagttaagtgaggtattgtgattgaatgattacatac |
| | | | atttttactgcttatgaaaaagattatggatttagaagaactgcaataaagaaatta |
| | | | actgaatattttattgcacag(SEQ ID NO 283) |
| 5 | ENSE000 | 171 | GTGCAGCTGCTAGCAAGGAAAATTATCTTTTCATATTTAAATCTGCTAGTGAATTCA |
| | 01314302 | | AAGAATGACCTGGCTGTGGCTTATATTCTCAATATTCCTGATAGAGGACTAGGAAGA |
| | | | GAAGCCTTCACTGATTTGAAACATGCTGCTCGAGAGAAACAAATGTCTATCTTTTTG |
| | | | (SEQ ID NO 284) |
| | Intron 5-6 | 1,120 | gtatggttgtgtgacatgttcaaaattaagactccttgcacagtggatccatgtatc |
| | | | ctaatatgcttgtaactaggatgaaacaacttttaagtggttaatttatgttctacc |
| | | | tctgtatttgaaaatccatggtattttaattttttaccatatcgttcaagtttggta |
| | | | aaggagaatttctctaaagcagtgtttcc....................accacact |
| | | | tgctagcatgaattctgcatggcatttgtttctttggttatttacttctgactcaaa |
| | | | gtgtgtcatgagccataaaatgtgcttcacttttaactgtatacccagaattatata |
| | | | gatttacttagctttatgaagtaaattacagtttaataaatcactggacataactta |
| | | | tccgtttgttttaattcatag (SEQ ID NO 285) |
| 6 | ENSE000 | 155 | GTGGCCACGTCTTTTATTAGAACAATAGAGCTTGGAGGGAAAGGATATGCACCACCA |
| | 03495299 | | CCATCAGATCCTTTAAGGACACATGTAAAGGGATTGTCTAATTTTATTAATTTCATT |
| | | | GACAAATTAGATGAGATTCTTGGAGAAATACCAAACCCAAG(SEQ ID NO 286) |
| | Intron 6-7 | 9,599 | gtaatgacttttcatatattacaaatatgtttttaatctatctttaaaactttacag |
| | | | aattgggtagaaataagggatattgaaatgaccaagcatcagaccaaacggtgaggc |
| | | | gaagaaacaaaatctagatgaataaattataatcacatgaccttacctcctaaattt |
| | | | ctgcctgtgattctgagttttggccaaag....................aaattact |
| | | | tgcttttagacaaacttttaaattttgtttctaatggtccttatctttttcttaaat |
| | | | gctttaactgtttgatatataagatcaaagctatattctttgaaaagtgttaactct |
| | | | gaaaattatattttcaagtctataagcatattataatttgcaatactgctatagagg |
| | | | caatctaatttctattttcag(SEQ ID NO 287) |
| 7 | ENSE000 | 184 | CATTGCAGGGGGTCAAATACTGTCAGTGATAAAGATGCAACTGATTAAAGGCCAAAA |
| | 03529925 | | CAGCAGGGATCCTTTTTGCAAAGCAATAGAGGAAGTTGCTCAGGATTTGGATTTGAG |
| | | | GATTAAAAATATTATCAATTCTCAAGAAGGTGTTGTAGCTCTTAGCACCACTGACAT |
| | | | CAGTCCTGCTCGG (SEQ ID NO 288) |
| | Intron 7-8 | 2,925 | gtaatgagctttttatttttcattatccttttatacaaatcattatctcttctattt |
| | | | attgtagaaatggtttattattgctacttaagtcagaagagtgtgtttcctgtgttc |
| | | | taaaatgatattgaaaatgtaagtttctcaataggattatgtaggattacatgttca |
| | | | acttgggtaaattatcacatatttgggcc....................gtgctttt |
| | | | ggaattggtaaagttgattatagcatggcttaaggaaaaatggtcacatgcctcaca |
| | | | gccacttcttatagctgatgttttattactttaacaatagatgaaagaggccagtgc |
| | | | cacagggaatttaaaagtatggccattcacccagctggggtgattattacatttacc |
| | | | taaaattattttttgtctcag(SEQ ID NO 289) |
| 8 | ENSE000 | 179 | cCAAAATCTCATGCCATAAACCATGGTACTGCATACTGTGGCAGAGATACTGTGAAA |
| | 03654471 | | GCCTTATTAGTTCTTTTGGACGAAGAAGCAGCTAATGCTCCTACCAAAAACAAAGCA |
| | | | GAGCTTTTATATGATGAGGAAAACACAATCCATCATCATGGAACGTCTATTCTTACA |
| | | | CTTTTTAG(SEQ ID NO 290) |
| | Intron 8-9 | 3,778 | gtaagttatgtggaagttatatgtgttataaatgttaactctagagaattataaaag |
| | | | aaatgtatgcttatggtaggaaacttagaaactacagaaattgtgaagaaagaaaat |
| | | | aaaagttaattaaagggagattgaacatttgaattcaaagaggttatatataaaaga |
| | | | agtaatattttatgagagtacagcctgag....................gttcggtg |
| | | | gattgggtgtattaaatgcatttttgacttaatggtatttttaatttatgatgagtt |
| | | | tatctgggaggtaaccccatcataagttgaggaaaaactagattgtattgggaatta |
| | | | agtttcaacgtgaatttggagggaaacaaacattccaaccatagcaataaatttttg |
| | | | ccttttttttttttgacatag(SEQ ID NO 291) |
| 9 | ENSE000 | 79 | GTCTCCCACACAGGTGAATAATTCGATAAAACCCCTAAGAGAACGCATCTGTGTGTC |
| | 03502613 | | AATGCAAGAGAAAAAAATTAAG (SEQ ID NO 292) |
| | Intron 9-10 | 12,684 | gtacaatttaatgcatgccatgaaaatagcaaacactgttttatttttagaagaaat |
| | | | aaatacatgaagctgagctgggtattgtaaatattgtaaacattgtacatgacacag |
| | | | taggaattaaatggaaaatgagttatgacagttgacgtacagagactttccttttat |
| | | | acagtcttgactaatctgtttctggtttg....................aagaacaa |
| | | | aaatgctagccataacaatgaatgagtttgcaaactatgaaatataaaaggttagag |
| | | | agaggttctcaatgagtaatagaatttctaagggaaatttttataccttgattacta |
| | | | tagagtgtgtgtctatctagatgaagaaataaaataatgaataaatttgcatatttt |
| | | | cttctttctttctgttactag(SEQ ID NO 293) |
| 10 | ENSE000 | 136 | ATGAAGCAAACTTTAATTAGATCCCAATTTGCTTGTACTTATAAAGATGACTACATG |
| | 03591981 | | ATAAGCAAGGATAATTGGAATAATGTTAATTTAGCATCAAAGCCTTTGTGTGTTCTT |
| | | | TACATGGAAAATGACCTTTCTG (SEQ ID NO 294) |
| | Intron 10-11 | 503 | gtaattgaccttatttgtgcaattaaataacaatttaattctagtctactaattagt |
| | | | tttaacttaagtaaaattaggttatttttgcaaagtactatgaaatgtaaagggtaa |
| | | | tatttttaaagacctcagtttatttaaaaagttaaatctaagtacaaatttatcgat |
| | | | aggaataggacacagtttgatttaactaa....................taactcag |
| | | | aggtgctatgataaaatgaaaagtaataggatagtaatgtagctatatttgtcaatc |
| | | | atattatttatagcattattttactttaaaaatttgaatattcgttagccattttta |
| | | | aagtttattgtaaagttctcgtaaatattaatactcaatatcatgtaattcctttcc |
| | | | cctttttatcttgcataacag(SEQ ID NO 295) |
| 11 | ENSE000 | 1,570 | AGGGTGTAAATCCATCTGTTGGAAGATCAACAATTGGAACGAGTTTTGGAAATGTTC |
| | 03571928 | | ATCTGGACAGAAGTAAAAATGAAAAAGTATCAAGAAAATCAACCAGTCAGACAGGAA |
| | | | ATAAAAGCTCAAAAAGGAAACAGGTGGATTTGGATGGTGAAAATATTCTCTGTGATA |
| | | | ATAGAAATGAACCACCTCAACATAAAAATGCTAAAATACCTAAGAAATCAAATGATT |
| | | | CACAGAATAGATTGTACGGCAAACTAGCTAAAGTAGCAAAAAGTAATAAATGTACTG |
| | | | CCAAGGACAAGTTGATTTCTGGCCAGGCAAAGTTAACTCAGTTTTTTAGACTATAAA |
| | | | TTTGTGTCTTATATGCTTTAGGTTTATGTATCTATAAACCATTCACCAAAGACATGC |
| | | | TTAATTTTTAAGAGATCAAGGTGTAAATTATGATGATTTATTATTTTGGTCTACAGT |
| | | | GTATGTAAGGTTAGTATGTTAAGCATTGTTTAAAAATACTAGTAAGTCATAATTATG |
| | | | CAGAATTTTCACAAAGTTTAATGCACAGAGAAAGCATATCATTTCAGTTACTGATAC |
| | | | ATCTTAACACTACTTTCTTTTAAAACAGACATTTAACATACACAAGTTATAGTAGCA |
| | | | GTATGGGCTTCTCCTCCCATTGGCAATTAAATGCTTTTATTTTCTTCTGAAAAGATG |
| | | | ATGTGGACCAACAGGTATCAGACTTGCCAACAAGGTCGGTAGACTCTTCCCAGCATA |
| | | | CATCTGAGCACTGAAGGAAGAAGAAAGTTTAAATTGTTTAAAGGACTATAATTATCA |
| | | | CACAAAATTTATTAAGAAAAAAAGAATGGATCTAGTATAACTAATTCTGAGTAAACC |
| | | | AAAATGATAATAATTAATTGTTGCTATTTAATCCCACATTTTTGGCAGGTGTAATTG |
| | | | AGCCATGGTCTTATTTGATTTTGTTATGATTGCATCCAAATTCACTTTAACTCAGAG |
| | | | TTCTGTTTAATGGTGGTAGGATGTAAGAATTGAATTTTGAAAAGACTACTCACTGTC |
| | | | AAAATCTCTCCTTCCTATAGGAAATTTAGCTGAGTTTTCTTCATCCCCAATTTCTCT |
| | | | CTTTTCTTGTGTTGATTCAGTATTCTGAACTCCATTCTCAGCTGGGAAAGCTACAGA |
| | | | TCCTTTTAGTGCAAGATAAGGTTTTATAGCCAGATTCAGTGGCAGACCATGATTTAA |
| | | | GAAATTATGTTTGGAGCCTGTGTTCTGTAAAGAGAAGGTTGATTTGGTTTTTAGCTA |
| | | | TCGTATTCGGAGTGGAACTATAATACAATTGTATAATATTCTTGTTGATCAATTCAA |
| | | | AGTTACTCTGCACTGTTTTTGACTTTTTAAAAATACCTTAGATGCAAATTTATAGGA |
| | | | GAAAAAACACTTTCAGATAAGAGGTGTTTGCTGGGATGGAAGAACTACCTGGCATGT |
| | | | AAGAAATATCGTCAGTCGTCCTAATGCATATTGTGACTGTTTGCATATACTTCTGTT |
| | | | TATAAAAGTATCAGTTTTACTTTTCAGAGGATTTGTAAGAATCATTTAAATTTTCAT |
| | | | TGAAATAAACGACAAGTCACATTGCCACTTA (SEQ ID NO 296) |

Coding sequence: (SEQ ID 165)
ATGGCTGTGTTTAATCAGAAGTCTGTCTCGGATATGATTAAAGAGTTTCGAAAAAATTGGCGTGCTCTTTGTAACTCTGAG
AGAACTACTCTATGTGGTGCAGACTCCATGCTCTTGGCATTGCAGCTTTCTATGGCGGAGAACAACAAACAGCACAGTGGA
GAATTTACAGTCTCTCTCAGTGATGTTTTATTGACATGGAAATACTTGCTCCATGAGAAATTGAACTTACCAGTTGAAAAC
ATGGACGTGACTGACCATTATGAGGACGTTAGGAAGATTTATGATGATTTCTTGAAGAACAGTAATATGTTAGACTGATTG
ATGTTTATCAAAAATGTAGGGCTTTGACTTCTAATTGTGAAAATTATAACACAGTATCTCCTAGTCAACTACTGGATTTTC
TGTCTGGCAAACAGTATGCAGTAGGTGATGAAACTGATCTTTCTATACCAACATCACCAACAAGTAAATACAACCGTGATA
ATGAAAAGGTGCAGCTGCTAGCAAGGAAAATTATCTTTTCATATTTAAATCTGCTAGTGAATTCAAAGAATGACCTGGCTG
TGGCTTATATTCTCAATATTCCTGATAGAGGACTAGGAAGAGAAGCCTTCACTGATTTGAAACATGCTGCTCGAGAGAAAC
AAATGTCTATCTTTTTGGTGGCCACGTCTTTTATTAGAACAATAGAGCTTGGAGGGAAAGGATATGCACCACCACCATCAG
ATCCTTTAAGGACACATGTAAAGGGATTGTCTAATTTTATTAATTTCATTGACAAATTAGATGAGATTCTTGGAGAAATAC
CAAACCCAAGCATTGCAGGGGGTCAAATACTGTCAGTGATAAAGATGCAACTGATTAAAGGCCAAAACAGCAGGGATCCTT
TTTGCAAAGCAATAGAGGAAGTTGCTCAGGATTTGGATTTGAGGATTAAAAATATTATCAATTCTCAAGAAGGTGTTGTAG
CTCTTAGCACCACTGACATCAGTCCTGCTCGGCCAAAATCTCATGCCATAAACCATGGTACTGCATACTGTGGCAGAGATA
CTGTGAAAGCCTTATTAGTTCTTTTGGACGAAGAAGCAGCTAATGCTCCTACCAAAAACAAAGCAGAGCTTTTATATGATG
AGGAAAACACAATCCATCATCATGGAACGTCTATTCTTACACTTTTTAGGTCTCCCACACAGGTGAATAATTCGATAAAAC
CCCTAAGAGAACGCATCTGTGTGTCAATGCAAGAGAAAAAAATTAAGATGAAGCAAACTTTAATTAGATCCCAATTTGCTT
GTACTTATAAAGATGACTACATGATAAGCAAGGATAATTGGAATAATGTTAATTTAGCATCAAAGCCTTTGTGTGTTCTTT
ACATGGAAAATGACCTTTCTGAGGGTGTAAATCCATCTGTTGGAAGATCAACAATTGGAACGAGTTTTGGAAATGTTCATC
TGGACAGAAGTAAAAATGAAAAAGTATCAAGAAAATCAACCAGTCAGACAGGAAATAAAAGCTCAAAAAGGAAACAGGTGG
ATTTGGATGGTGAAAATATTCTCTGTGATAATAGAAATGAACCACCTCAACATAAAAATGCTAAAATACCTAAGAAATCAA
ATGATTCACAGAATAGATTGTACGGCAAACTAGCTAAAGTAGCAAAAAGTAATAAATGTACTGCCAAGGACAAGTTGATTT
CTGGCCAGGCAAAGTTAACTCAGTTTTTTAGACTATAA
Skipping of PARPBP exon 6 produces a frame-shift which generates a novel 15
aminoacid-long peptide at the C-terminal of PARPBP protein and a premature
stop-codon (SEQ ID NO 166)
Met A V F N Q K S V S D Met I K E F R K N W R A L C N S E R T T L C G A D S Met L
L A L Q L S Met A E N N K Q H S G E F T V S L S D V L L T W K Y L L H E K L N L P
V E N Met D V T D H Y E D V R K I Y D D F L K N S N Met L D L I D V Y Q K C R A L
T S N C E N Y N T V S P S Q L L D F L S G K Q Y A V G D E T D L S I P T S P T S K
Y N R D N E K V Q L L A R K I I F S Y L N L L V N S K N D L A V A Y I L N I P D R
G L G R E A F T D L K H A A R E K Q Met S I F L H C R G S N T V S D K D A T D
Stop
SEQ ID NO: 192: H C R G S N T V S D K D A T D

### Example 7. A Novel Splice Junction Between Exon 27 and Exon 29 in CEMIP mRNA is Induced by Specific Oligonucleotides, an Extended Screen to Identify More Efficacious Compounds Compared to Example 2. (Results are Shown in Table 1)

To induce a novel splice junction between exon 27 and exon 29 based on the transcript isoform CEMIP-201 transcript (ENST00000220244.7), we designed oligonucleotides to target the CEMIP pre-mRNA at sites flanking or overlapping CEMIP exon 28 sequence (Exon 28 is the underlined sequence—the non-underlined sequences are upstream or down-stream intron sequence.

(SEQ ID NO 297)
catgcccactttctccagtttgctctctccctctggtctaattggtttct
tctcacacagTTCCATAGTGCTTATGGCATCAAAGGGAAGATACGTCTCC
AGAGGCCCATGGACCAGAGTGCTGGAAAAGCTTGGGGCAGACAGGGGTCT
CAAGTTGAAAGgtaagggtttgaactggggtttaaactgaccccaaaacc
agagaaggcaaatgc

4×10³ Colo-205 cells were seeded in 96-well plate format and cultured in RPMI media supplemented with 10% FBS and 1% pen/strep. 82 different oligonucleotides targeting CEMIP pre-mRNA sequence (or vehicle only, PBS) were added to the cells at 5 μM and 25 μM final concentration. After 4 days, cells were harvested, and RNA was extracted using RNeasy Mini extraction kit (Qiagen). cDNA was generated using iScript Advanced cDNA synthesis kit and processed for ddPCR analysis (Biorad). The expression level of CEMIP mRNA containing the induced exon 27/exon 29 splice junction is represented as the percentage of total CEMIP transcripts. QuantaSoft software (Biorad) was used for analysis. The following probe-based assays were used to detect the splice event: CEMIP exon 27/exon 29 junction (forward primer GGAACTCCATTCTGCAAGG (SEQ ID NO 173), reverse primer CCTCAGTGTCCAGTGTCA (SEQ ID NO 174), /56-FAM/CCA TCCCTG/ZEN/ACAAAGCAAATGGCATTC/3IABkFQ/(SEQ ID NO 175)); total CEMIP (forward primer CTCGGTGCTGAGGTTAACTC (SEQ ID NO 176), reverse primer TCAGACTAAAGGTGGGGAGAA (SEQ ID NO 177), probe /5HEX/TCAGACCTC/ZEN/TGGAAAGCTCACCCA/3IABkFQ/(SEQ ID NO 172)). Compared to the wild-type CEMIP mRNA, the induced exon 27/exon 29 junction generates a frame shift in the mRNA coding sequence with a subsequent novel 21 aminoacid-long polypeptide at CEMIP C-terminal region (K A N G I R W L Q R Q L P A H L G D T G H, SEQ ID NO 189).

The following LNA containing oligonucleotides were used to induce alteration in splicing events. All internucleoside linkages are phopshorothionate. Upper and lower case indicate LNA and DNA nucleobases, respectively. The oligos and results are shown in the table below.

The data shows the percentiles of CEMIP mRNAs containing the novel exon 27/exon 29 junction.

| | | | | % of exon | |
|---|---|---|---|---|---|
| | | | | 27/exon 29 | |
| | | | | junction | |
| Oligo No | Compound | Sequence | SEQ ID | 5 uM | 25 uM |
| O165 | TtaGAcCAgAGgGA | TTAGACCAGAGGGA | 193 | 0.0 | 0.1 |
| O166 | TaGAccAGaGGgAG | TAGACCAGAGGGAG | 194 | 0.0 | 0.0 |
| O167 | AGacCAgaGGgaGA | AGACCAGAGGGAGA | 195 | 0.0 | 0.1 |
| O168 | GAccAGaGgGAgAG | GACCAGAGGGAGAG | 196 | 0.1 | 0.3 |
| O169 | ACcAGaGgGAgaGA | ACCAGAGGGAGAGA | 197 | 0.0 | 0.0 |
| O170 | CCagAgGgaGAgAG | CCAGAGGGAGAGAG | 198 | 0.1 | 0.4 |
| O171 | CaGAgGgaGAgaGC | CAGAGGGAGAGAGC | 199 | 0.0 | 0.0 |
| O172 | AgaGgGAgaGAgCA | AGAGGGAGAGAGCA | 200 | 0.0 | 0.1 |
| O173 | GAgGGaGAgAGcAA | GAGGGAGAGAGCAA | 201 | 0.0 | 0.0 |
| O174 | AGgGAgAGaGCaAA | AGGGAGAGAGCAAA | 202 | 0.0 | 0.2 |
| O175 | GGgAGaGAgCAaAC | GGGAGAGAGCAAAC | 203 | 0.0 | 0.1 |
| O176 | GGaGAgaGCaAaCT | GGAGAGAGCAAACT | 204 | 0.2 | 0.0 |
| O177 | GAgAGaGCaAAcTG | GAGAGAGCAAACTG | 205 | 0.1 | 0.0 |
| O178 | CTTACCTTTC | CTTACCTTTC | 206 | 0.0 | 0.1 |
| O179 | CCtTaCCtTtCA | CCTTACCTTTCA | 207 | 0.6 | 1.4 |
| O180 | AGttCAaACcCTtaCC | AGTTCAAACCCTTACC | 208 | 20.9 | 57.6 |
| O181 | TtCAaAcCCtTaCcTT | TTCAAACCCTTACCTT | 209 | 14.2 | 41.4 |
| O182 | CAaaCCcTtaCCttTC | CAAACCCTTACCTTTC | 210 | 4.9 | 21.1 |
| O183 | AaCCcTTaCCtTTcAA | AACCCTTACCTTTCAA | 211 | 0.4 | 1.6 |
| O184 | CCcTTacCTttCAaCT | CCCTTACCTTTCAACT | 212 | 2.7 | 8.5 |
| O185 | CTtaCCttTCaACtTG | CTTACCTTTCAACTTG | 213 | 6.9 | 20.5 |
| O186 | TaCCtTtCAaCTtgAG | TACCTTTCAACTTGAG | 214 | 1.5 | 3.7 |
| O187 | CatTCaaCTtGAgAC | CCTTTCAACTTGAGAC | 215 | 1.1 | 3.5 |
| O188 | AGttCAaACccTTaCcTT | AGTTCAAACCCTTACCTT | 216 | 17.5 | 47.0 |
| O189 | TTcAaaCCcTTacCTtTC | TTCAAACCCTTACCTTTC | 217 | 7.0 | 23.1 |
| O190 | CAaaCCcTTaCCatcAA | CAAACCCTTACCTTTCAA | 218 | 2.1 | 7.3 |
| O191 | AAcCCttAcCTttCAaCT | AACCCTTACCTTTCAACT | 219 | 4.3 | 12.5 |
| O192 | CCcTtACcTTtcAActTG | CCCTTACCTTTCAACTTG | 220 | 2.3 | 6.4 |
| O193 | CTtACcTTtCAaCTtgAG | CTTACCTTTCAACTTGAG | 221 | 1.7 | 4.8 |
| O194 | TacCTttCAaCTtGAgAC | TACCTTTCAACTTGAGAC | 222 | 1.0 | 1.7 |
| O195 | AGttCaaAccCTtAccTtTC | AGTTCAAACCCTTACCTTTC | 223 | 48.0 | 79.4 |
| O196 | TTcAAaCCcTTacCTtTcAA | TTCAAACCCTTACCTTTCAA | 224 | 3.1 | 7.9 |
| O197 | CaaACccTTacCatCAaCT | CAAACCCTTACCTTTCAACT | 225 | 4.8 | 16.3 |
| O198 | AacCatACcTTtcAActTG | AACCCTTACCTTTCAACTTG | 226 | 2.9 | 9.2 |
| O199 | CcCTtAccTTtcAActTgAG | CCCTTACCTTTCAACTTGAG | 227 | 2.2 | 8.5 |
| O200 | CTtAcCTttCAacTTgAgAC | CTTACCTTTCAACTTGAGAC | 228 | 1.6 | 3.0 |
| O201 | GGgTCaGTtTAaAC | GGGTCAGTTTAAAC | 229 | 0.0 | 0.1 |
| O202 | GtCAgTtTAaAcCC | GTCAGTTTAAACCC | 230 | 0.0 | 0.0 |
| O203 | CAgTtTAaaCCcCA | CAGTTTAAACCCCA | 231 | 0.1 | 0.0 |
| O204 | GTtTAaACcCCaGT | GTTTAAACCCCAGT | 232 | 0.0 | 0.1 |
| O205 | TTaAaCCcCAgtTC | TTAAACCCCAGTTC | 233 | 0.1 | 0.1 |
| O206 | AAaCCcCAgTTcAA | AAACCCCAGTTCAA | 234 | 0.1 | 0.0 |
| O207 | ACcCCaGTtCAaAC | ACCCCAGTTCAAAC | 235 | 0.0 | 0.0 |
| O208 | CCcAGtTCaaAcCC | CCCAGTTCAAACCC | 236 | 0.0 | 0.3 |
| O209 | CAgTtCAaaCCcTT | CAGTTCAAACCCTT | 237 | 3.9 | 15.5 |
| O210 | GTtCAaAcCCttAC | GTTCAAACCCTTAC | 238 | 39.4 | 80.5 |
| O211 | TCaAAcCCtTAcCT | TCAAACCCTTACCT | 239 | 7.2 | 23.7 |
| O212 | AAaCCcTTaCCtTT | AAACCCTTACCTTT | 240 | 0.5 | 2.3 |
| O213 | AcCCtTAccTTtCA | ACCCTTACCTTTCA | 241 | 3.4 | 11.7 |
| O214 | CCtTaCCttTCaAC | CCTTACCTTTCAAC | 242 | 1.8 | 4.9 |
| O215 | TTaCCtTtCAacTT | TTACCTTTCAACTT | 243 | 1.5 | 7.3 |
| O216 | ACcTTtCAaCTtGA | ACCTTTCAACTTGA | 244 | 0.6 | 1.9 |
| O217 | CTtTCaACtTGaGA | CTTTCAACTTGAGA | 245 | 0.2 | 0.4 |
| O218 | TtCAaCtTGaGaCC | TTCAACTTGAGACC | 246 | 0.1 | 0.3 |
| O219 | CAaCTtGAgACcCC | CAACTTGAGACCCC | 247 | 0.2 | 0.0 |
| O220 | AcTTgAGacCCcTG | ACTTGAGACCCCTG | 248 | 0.0 | 0.0 |
| O221 | TTgaGAccCCtgTC | TTGAGACCCCTGTC | 249 | 0.0 | 0.1 |
| O222 | GAgAccCctGTcTG | GAGACCCCTGTCTG | 250 | 0.1 | 0.2 |
| O223 | GacCccTgtCTgCC | GACCCCTGTCTGCC | 251 | 0.1 | 0.0 |
| O224 | CccCTgTctGccCC | CCCCTGTCTGCCCC | 252 | 0.1 | 0.2 |
| O225 | CCtGTcTgCCccAA | CCTGTCTGCCCCAA | 253 | 0.1 | 0.0 |
| O226 | TgtCTgCCccAaGC | TGTCTGCCCCAAGC | 254 | 0.1 | 0.2 |
| O227 | TcTgCCcCAaGcTT | TCTGCCCCAAGCTT | 255 | 0.0 | 0.0 |
| O228 | TgCCcCAagCTtTT | TGCCCCAAGCTTTT | 256 | 0.1 | 0.1 |
| O229 | CCcCAaGcTTttCC | CCCCAAGCTTTTCC | 257 | 0.6 | 1.2 |
| O230 | CCaAGcTTtTCcAG | CCAAGCTTTTCCAG | 258 | 0.1 | 0.6 |
| O231 | AaGCtTTtCCagCA | AAGCTTTTCCAGCA | 259 | 0.1 | 0.0 |
| O232 | GCttTTccAGcaCT | GCTTTTCCAGCACT | 260 | 0.0 | 0.0 |
| O233 | TTttCcaGCaCtCT | TTTTCCAGCACTCT | 261 | 0.2 | 0.7 |
| O234 | TtCCagCAcTctGG | TTCCAGCACTCTGG | 262 | 0.2 | 0.1 |
| O235 | CCaGcActCTggTC | CCAGCACTCTGGTC | 263 | 0.1 | 0.1 |
| O236 | AGcActCTgGTcCA | AGCACTCTGGTCCA | 264 | 0.3 | 0.6 |
| O237 | CActCTgGTcCaTG | CACTCTGGTCCATG | 265 | 0.1 | 0.5 |
| O238 | CtCTgGTccATgGG | CTCTGGTCCATGGG | 266 | 0.1 | 0.5 |
| O239 | CTgGTccATgGgCC | CTGGTCCATGGGCC | 267 | 0.0 | 0.0 |
| O240 | GgTccATgGGccTC | GGTCCATGGGCCTC | 268 | 0.0 | 0.0 |
| O241 | TccATgGgCCtcTG | TCCATGGGCCTCTG | 269 | 0.5 | 1.2 |
| O242 | CAtgGgCctCTgGA | CATGGGCCTCTGGA | 270 | 0.6 | 4.5 |
| O243 | TggGcCTcTGgaGA | TGGGCCTCTGGAGA | 271 | 0.8 | 0.8 |
| O244 | GgCctCTgGAgaCG | GGCCTCTGGAGACG | 272 | 0.1 | 0.1 |
| O245 | CCtcTGgaGAcgTA | CCTCTGGAGACGTA | 273 | 1.7 | 4.5 |
| O246 | TCtGGaGAcGTaTC | TCTGGAGACGTATC | 274 | 0.2 | 0.5 |

### Example 8. Next Generation Sequencing Verification of Precise Nucleotide Junction Between Exon 27 and Exon 29 in CEMIP mRNA Induced by O195

The precise nucleotide junction between CEMIP exon 27 and CEMIP exon 29 was verified by Next Generation Sequencing: 1.5×10⁶ Colo-205 cells were seeded in 6-well plate format and incubated with vehicle (n=2) or O195 (n=2) at 50 μM for 4 days. Cells were harvested, and RNA was extracted using RNeasy Mini extraction kit (Qiagen). cDNA synthesis was performed, followed by enrichment for CEMIP cDNA using capture probe enrichment. RNAseq was then performed using Illumina NGS sequencing. The sequencing verified that the predicted junction was indeed induced by O195. The induction of the novel splice junction by antisense oligonucleotide treatment was quantitated as the ratio between the reads matching the junction exon 27-exon 29 junction and the reads matching junction the wild type exon 27-exon 28 junction. O195 was able to induce exon 28 skipping with 84% efficiency, while vehicle treated samples showed undetected exon 28 skipped form.

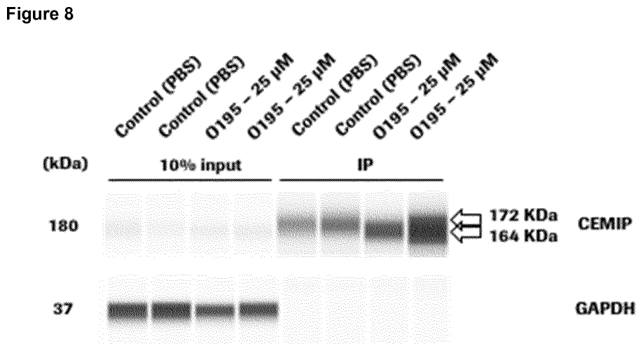

### Example 9. Shift in Molecular Weight of CEMIP Protein Induced by O195 (See FIG. 8)

1.5×10⁶ Colo-205 cells were seeded in 6-well plate format and incubated with vehicle (n=2) or O195 (n=2) at 25 μM for 4 days. Cells were harvested, washed with PBS, and lysed in 500 μl of IP lysis buffer (#87787, ThermoFisher) containing protease and phosphatase inhibitors (#78446, ThermoFisher). 10% of lysate was saved as input control, while the rest was incubated with 2 μg of anti-CEMIP antibody (NBP1-58029, Novus Biologicals) and kept in rotation overnight at 4° C. 10 μl of protein A/G agarose beads slurry (#20421, ThermoFisher) was added to the lysate and kept in rotation at 4° C. for 1.5 hours. Beads were washed three times with ice-cold IP lysis buffer before resuspension in WES loading buffer for immunoblotting analysis. GAPDH signal was used as loading control, and to confirm immunoprecipitation efficiency. CEMIP was readily detectable and successfully pulled down in all the biological samples. This indicates that manipulation of CEMIP mRNA splicing does not impairs protein translation. Moreover, treatment with O195 led to an apparent measured reduction of CEMIP molecular weight from 172 KDa to 164 KDa, when compared to the vehicle treated samples. These results suggest that CEMIP protein may present a novel C terminal sequence upon treatment with O195.

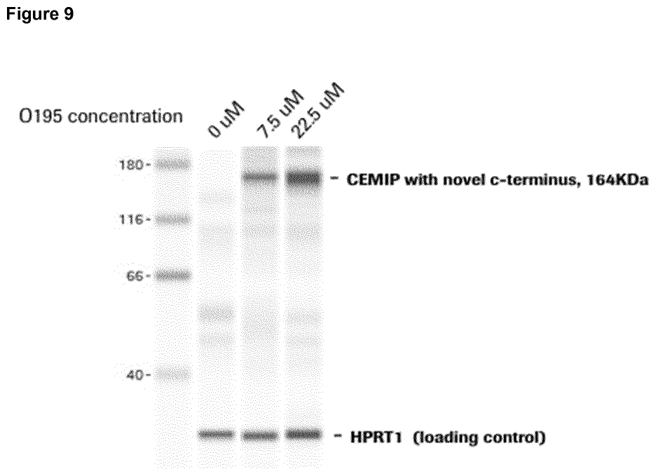

### Example 10. Antibody Recognizing the Production of a Novel c-Terminus of CEMIP Following Induction of Exon 27-Exon 29 Junction of CEMIP mRNA (See FIG. 9)

A polyclonal antibody targeting the novel c-terminus of CEMIP was generating by immunizing rabbits with peptide corresponding to the predicted novel c-terminus of CEMIP (K ANG IR W L Q R Q L P A H L G D T G H, 189). 4×10³ Colo-205 cells were seeded in 96-well plate format and cultured in RPMI media supplemented with 10% FBS and 1% pen/strep. Cells were incubated with Oligo 195 (223) at a concentration of 7.5 uM and 22.5 uM. Cells were harvested after 4 days in 50 uL RIPA buffer (Themo scientific). Cell lysate was analyzed by capillary electrophoresis using the Wes system from protein simple according to manufactures instructions. An antibody targeting HPRT1 was included as a loading control. (Abcam ab109021). The results is displayed in FIG. 9, showing that treatment of cells with oligo 195 induces a strong band with a size around 164 kDa. This corresponds to the size of the predicted truncated CEMIP protein with the novel c-terminus.

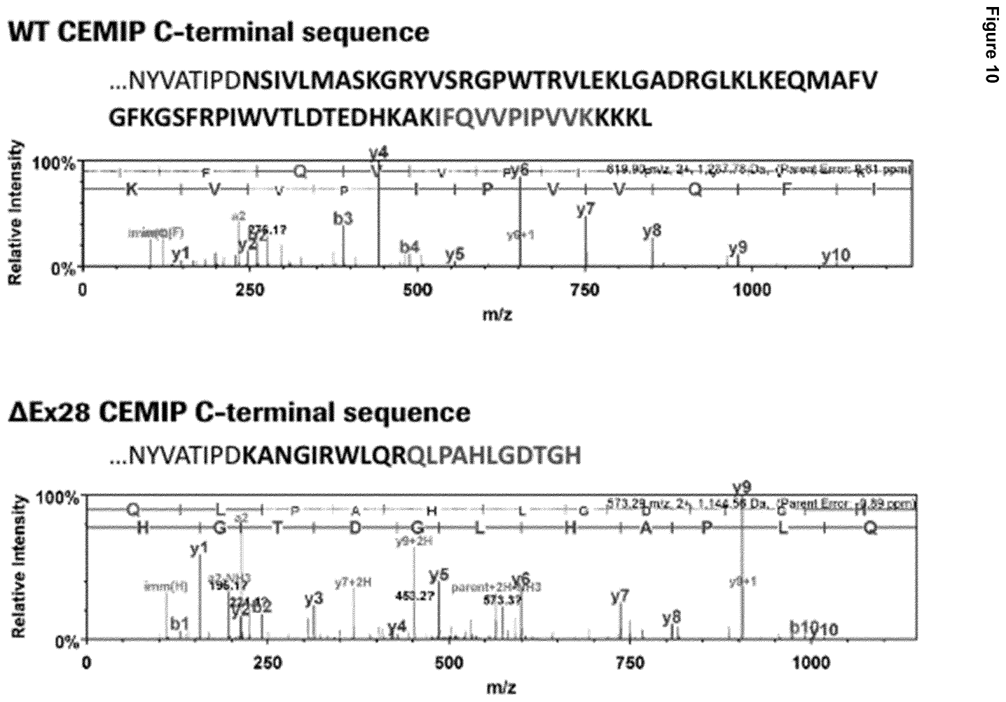

### Example 11 Mass Spectrometry Verification of Novel c-Terminus Peptide Sequence Induced by O195 (See FIG. 10)

CEMIP immunoprecipitated samples (as described in Example 9) were submitted for mass spectrometry analysis. As expected, both vehicle treated and O195-treated samples contained the wild-type CEMIP protein sequence. Strikingly, only O195-treated samples also presented the novel C-terminus peptide which is predicted by exon 28 skipping. These data demonstrate that the exon 28 skipped isoform induced by O195 is efficiently translated and generates the predicted novel C-terminus.

### Sample Preparation

Immunoprecipitated samples were boiled in 60 μL of 1.5× LDS buffer for 15 minutes. Half of each elution was processed by SDS-PAGE using a 10% Bis-Tris NuPAGE (Invitrogen) with the MES buffer system. The mobility region was excised into 10 equal sized segments and in-gel digestion was performed on each using a robot (ProGest, DigiLab) with the following protocol:
Washed with 25 mM ammonium bicarbonate followed by acetonitrile.
Reduced with 10 mM dithiothreitol at 60° C. followed by alkylation with 50 mM iodoacetamide at RT.
Digested with sequencing grade trypsin (Promega) at 37° C. for 4 h.
Quenched with formic acid and the supernatant was analyzed directly without further processing.

### Mass Spectrometry

Half of each gel digest was analyzed by nano LC-MS/MS with a Waters NanoAcquity HPLC system interfaced to a ThermoFisher Fusion Lumos mass spectrometer. Peptides were loaded on a trapping column and eluted over a 75 μm analytical column at 350 nL/min; both columns were packed with Luna C18 resin (Phenomenex). The mass spectrometer was operated in data-dependent mode, with the Orbitrap operating at 60,000 FWHM and 15,000 FWHM for MS and MS/MS respectively. The instrument was run with a 3s cycle for MS and MS/MS.

5 hrs of instrument time was employed per sample.

### Data Processing

Data were searched using a local copy of Mascot with the following parameters:

Enzyme: Trypsin/P

Database: SwissProt Human plus the custom sequence* (concatenated forward and reverse plus common contaminants)

Fixed modifications: Carbamidomethyl (C)

Variable modifications: Oxidation (M), Acetyl (N-term), Pyro-Glu (N-term Q), Deamidation (N,Q)

Mass values: Monoisotopic

Peptide Mass Tolerance: 10 ppm

Fragment Mass Tolerance: 0.02 Da

Max Missed Cleavages: 2

1. A method for engineering a peptide epitope in a cell, said method comprising administration of an effective amount of an RNA modifying oligonucleotide to the cell, wherein the RNA modifying oligonucleotide targets a target RNA to modulate the coding sequence of the target RNA to produce an aberrant RNA transcript encoding an aberrant polypeptide containing the peptide epitope.

2. A method for engineering a peptide epitope in a cell according to claim 1, wherein the RNA modifying oligonucleotide is either a splice modulating oligonucleotide or is a RNA editing oligonucleotide.

3. A method for engineering a peptide epitope in a cell, wherein the RNA modifying oligonucleotide is a splice modulating oligonucleotide, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope.

4. The method according to claim 3, wherein either:
a) the splice modulating oligonucleotide modulates the of splicing of the target RNA to produce an aberrant RNA transcript introduced by the modulated splicing event, wherein the aberrant RNA transcript encodes an internal polypeptide deletion, to produce an aberrant polypeptide comprising an aberrant peptide sequence at the modulated splicing event, to produce the peptide epitope; and/or
b) the splice modulating oligonucleotide modulates the splicing of the target RNA to produce an aberrant RNA transcript introduced by the modulated splicing event, wherein the aberrant RNA transcript encodes one or more codons from an intronic region of the target RNA, to produce an aberrant polypeptide comprising an aberrant peptide sequence which includes at least one or more peptide(s) encoded by the one or more codons originating from the intronic region, to produce the peptide epitope; and/or
c) the splice modulating oligonucleotide modulates the of splicing of the target RNA, to produce an aberrant RNA transcript comprising a codon frame shift introduced by the modulated splicing event, wherein the aberrant RNA transcript produces a polypeptide with a C-terminal region of at least 1 amino acid, which is transcribed from the region of the aberrant RNA transcript at or 3′ to the codon frame shift.
d) A method for engineering a peptide epitope in a cell, said method comprising administration of an effective amount of a splice modulating oligonucleotide to the cell, wherein the splice modulating oligonucleotide targets a target RNA to modulate the splicing of the target RNA to produce an aberrant RNA transcript encoding an aberrant polypeptide containing the peptide epitope.

5. The method according to claim 1, wherein the cell is a cancer cell.

6. The method according to claim 1, wherein the method is an in vitro method or an in vivo method.

7. The method according to claim 1, wherein the target RNA is a RNA which is over-expressed in the cancer cell.

8. The method according to claim 1, wherein the peptide epitope is secreted from the cell.

9. The method according to claim 1, wherein the peptide epitope is presented on the cell as a MHC Class I or II molecule.

10. The method according to claim 1, wherein the polypeptide containing the peptide epitope further comprises a membrane binding domain.

11. The method according to claim 1, wherein the RNA is a pre-mRNA selected from the group consisting of: CEMIP, ETV4, LRG5, NOX1, FOXP3, IGF2BP3, MAGE-A4, NY-ESO-1, EWSR1, FUS, PARPBP and SS18.

12. The method according to, wherein either:
a. the pre-mRNA is CEMIP, wherein the antisense oligonucleotide comprises a contiguous nucleotide sequence of at least 10 nucleotides, which have 100% identity with a sequence selected from 1-82, or 193-274; or
b. the pre-mRNA is ETV4, wherein the antisense oligonucleotide comprises a contiguous nucleotide sequence of at least 10 nucleotides, which have 100% identity with a sequence selected from 83-164.

13. The method according to claim 1, wherein the RNA modifying oligonucleotide comprises 2′ sugar modified nucleosides selected from: 2′ sugar modified nucleosides independently selected from 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside, and LNA nucleosides.

14. The method according to claim 1, wherein the RNA modifying oligonucleotide comprises modified internucleoside linkages.

15. The method according to claim 1, wherein the RNA modifying oligonucleotide is a splice modulating oligonucleotide which is a 2′-O-MOE oligonucleotide.

16. The method according to claim 1, wherein the RNA modifying oligonucleotide is a splice modulating oligonucleotide which is a LNA oligonucleotide.

17. The method according to claim 1, wherein the RNA modifying oligonucleotide is a splice modulating oligonucleotide which is a morpholino oligonucleotide.

18. A method of immune modulating a target cell in a subject, said method comprising the steps of:
a. Vaccinate the subject with an agent comprising a peptide epitope, or encoding peptide epitope;
b. Administer a RNA modifying oligonucleotide to the subject, wherein the RNA modifying oligonucleotide targets a target RNA in a target cell in the subject, and modulates the target RNA to produce an aberrant RNA transcript encoding an aberrant polypeptide containing the peptide epitope;
c. to trigger or enhance the immune response by the subject to the peptide epitope;
d. wherein step a. and step b: may be in the order of step a. and then step b., or step b. and then step a., or step a. and step b. are performed simultaneously.

19. A method of immune modulating a target cell in a subject, said method comprising the steps of:
a. administer a RNA modifying oligonucleotide to the subject, wherein the RNA modifying oligonucleotide targets a target RNA in a target cell in the subject, and modulates the RNA to produce an aberrant RNA transcript encoding an aberrant polypeptide containing a peptide epitope;
b. administer an antibody to the subject, wherein the antibody is specific for the peptide epitope
c. to trigger or enhance the immune response by the subject to the peptide epitope; wherein step a. and step b: may be in the order of step a. and then step b., or step b. and then step a., or step a. and step b. are performed simultaneously.

20. A method of immune modulating a target cell in a subject, said method comprising the step of administering a RNA modifying oligonucleotide to the subject, wherein the RNA modifying oligonucleotide targets a target RNA in a target cell in the subject, to produce an aberrant RNA transcript encoding an aberrant polypeptide containing the peptide epitope; to trigger or enhance the immune response by the subject to the peptide epitope.

21. The method according to claim 18, wherein the method further comprises the step of administering a check point inhibitor to the subject, selected from a PDL1 inhibitor, a PD1 inhibitor or CTLA-4 inhibitor.

22. A method of immunotherapy treatment of a disease in a subject, said method comprising the steps of
a. administer a RNA modifying oligonucleotide to the subject, wherein the RNA modifying oligonucleotide targets a target RNA in the target cell in the subject, to produce an aberrant RNA transcript encoding an aberrant polypeptide containing a peptide epitope;
b. administer an immunotherapy antibody to the subject, wherein the immunotherapy antibody is specific for the peptide epitope;
c. to trigger or enhance the immune response by the subject to the peptide epitope; wherein step a. and step b: may be in the order of step a. and then step b., or step b. and then step a., or step a. and step b. are performed simultaneously.

23. The method according to claim 18, wherein the method is a method of treatment for cancer.

24. The method according to claim 18, wherein the cell is a cancer cell, selected from: a tumor, lung cancer, breast cancer, colon cancer cell, mestastizised colon cancer cell, or a mestastizised colon cancer cell in the liver.

25.-32. (canceled)

33. An antisense oligonucleotide capable of modulating the splicing of CEMIP pre-mRNA, wherein the antisense oligonucleotide comprises a contiguous nucleotide sequence of at least 10 nucleotides which have 100% identity with a sequence selected from SEQ ID NO 1-82, or 193-274.

34. An antisense oligonucleotide capable of modulating the splicing of ETV4 pre-mRNA, wherein the antisense oligonucleotide comprises a contiguous nucleotide sequence of at least 10 nucleotides which have 100% identity with a sequence selected from SEQ ID NO 83-164.

35. A splice modulating antisense oligonucleotide comprising or consisting the sequence 1-164, or a compound selected from the group consisting of O1-O164, or O165-O246.

36. A vaccine or immunotherapy agent which comprises the peptide epitope, selected from the groups 188, 189, 190, 191 or 192.

37. A polypeptide which is or comprises a peptide selected from the groups 188, 189, 190, 191 or 192.

38. A polypeptide which is or comprises a peptide selected from the groups 188, 189, 190, 191 or 192, for use in medicine.
